(19) United States
(12) Patent Application Publication
(10) Pub. No.: US 2025/0034238 A1
(43) Pub. Date: Jan. 30, 2025

(54) ANTI-TAUC3 ANTIBODIES AND USES THEREOF

(71) Applicant: TauC3 Biologics Limited, London (GB)

(72) Inventors: Daniel Chain, New York, NY (US); Preeti Bakrania, Stevenage (GB); Seema Patel, Stevenage (GB)

(21) Appl. No.: 18/914,205

(22) Filed: Oct. 13, 2024

Related U.S. Application Data
(63) Continuation of application No. 17/477,687, filed on Sep. 17, 2021, now Pat. No. 12,162,930.
Application No. 17/477,687 is a continuation of application No. 16/838,235, filed on Apr. 2, 2020, now Pat. No. 11,155,609.
(60) Provisional application No. 62/829,774, filed on Apr. 5, 2019.

(51) Int. Cl. C07K 16/18

(52) U.S. Cl. CPC C07K 16/18; C07K 2317/24; C07K 2317/565; C07K 2317/92

(57) ABSTRACT

Anti-TauC3 antibodies that are at least several orders of magnitude more specific for TauC3 than for full length tau (2N4R) are described. Also described are methods of using anti-TauC3 antibodies.

This is a continuation of U.S. patent application Ser. No. 17/477,687, filed Sep. 17, 2021, which is a continuation of U.S. patent application Ser. No. 16/838,235, filed Apr. 2, 2020, which claims the benefit of U.S. Provisional Application No. 62/829,774, filed on Apr. 5, 2019, the disclosures of which are hereby incorporated by reference.

### REFERENCE TO AN ELECTRONIC SEQUENCE LISTING

The contents of the electronic sequence listing (350011_1002CON2_SequenceListing_13Oct2024.xml; Size: 198,000 bytes; and Date of Creation: Oct. 13, 2024) is herein incorporated by reference in its entirety.

### BACKGROUND OF THE INVENTION

Tau protein is a microtubule associated protein that distributes mainly to axons and modulates the assembly, spatial organization and behavior of microtubules (MT) in neurons. Tau protein is encoded by a single gene located on chromosome 17.

There are six known isoforms of Tau protein. These isoforms differ from each other by the presence or absence of 29- or 58-amino acid inserts in the amino-terminal region and by the addition or deletion of tandem repeats (which can be repeated either 3 or 4 times) in a carboxy-terminal region of tau, which is referred to as microtubule binding domain. The microtubule binding domain region is composed of imperfect repeats of 31 or 32 amino acid residues. The longest tau protein isoform, (2N4R), is 441 amino acids in length and contains four repeats (R1, R2, R3 and R4) and two inserts. The smallest tau isoform contains 352 amino acid residues with three tandem repeats (R1, R3 and R4) in the microtubule binding domain and no amino terminal inserts. The amino acid sequences corresponding to the isoforms of the human tau protein are provided in SEQ ID NOs: 1-6.

SEQ ID NO: 1, the longest tau isoform, htau40, containing two N-terminal inserts and four microtubule binding (2N4R) domains, is as follows:

| SEQ ID NO: 1, the longest tau isoform, htau40, containing two | |
|---|---|
| N-terminal inserts and four microtubule binding (2N4R) | |
| domains, is as follows: | |
| MAEPRQEFEV MEDHAGTYGL GDRKDQGGYT MHQDQEGDTD AGLKESPLQT PTEDGSEEPG | 60 |
| SETSDAKSTP TAEDVTAPLV DEGAPGKQAA AQPHTEIPEG TTAEEAGIGD TPSLEDEAAG | 120 |
| HVTQARMVSK SKDGTGSDDK KAKGADGKTK IATPRGAAPP GQKGQANATR IPAKTPPAPK | 180 |
| TPPSSGEPPK SGDRSGYSSP GSPGTPGSRS RTPSLPTPPT REPKKVAVVR TPPKSPSSAK | 240 |
| SRLQTAPVPM PDLKNVKSKI GSTENLKHQP GGGKVQIINK KLDLSNVQSK CGSKDNIKHV | 300 |
| PGGGSVQIVY KPVDLSKVTS KCGSLGNIHH KPGGGQVEVK SEKLDFKDRV QSKIGSLDNI | 360 |
| THVPGGGNKK IETHKLTFRE NAKAKTDHGA EIVYKSPVVS GDTSPRHLSN VSSTGSIDMV | 420 |
| DSPQLATLAD EVSASLAKQG L | 441 |
| SEQ ID NO: 2 contains two N-terminal inserts and three | |
| microtubule-binding domains (2N3R) as follows: | |
| MAEPRQEFEV MEDHAGTYGL GDRKDQGGYT MHQDQEGDTD AGLKESPLQT PTEDGSEEPG | 60 |
| SETSDAKSTP TAEDVTAPLV DEGAPGKQAA AQPHTEIPEG TTAEEAGIGD TPSLEDEAAG | 120 |
| HVTQARMVSK SLDGTGSDDK KAKGADGKTK IATPRGAAPP GQKGQANATR IPAKTPPAPK | 180 |
| TPPSSGEPPK SGDRSGYSSP GSPGTPGSRS RTPSLPTPPT REPKKVAVVR TPPKSPSSAK | 240 |
| SRLQTAPVPM PDLKNVKSKI GSTENLKHQP GGGKVQIVYK PVDLSKVTSK CGSLGNIHHK | 300 |
| PGGGQVEVKS EKLDFKDRVQ SKIGSLDNIT HVPGGGNKKI ETHKLTFREN AKAKTDHGAE | 360 |
| IVYKSPVVSG DTSPAHLSNV SSTGSIDMVD SPQLATLADE VSASLAKQGL | 410 |
| SEQ ID NO:3 contains one N-terminal insert and four microtubule- | |
| binding domains (1N4R) as follows: | |
| MAEPRQEFEV MEDHAGTYGL GDRKDQGGYT MHQDQEGDTD AGLKESPLQT PTGDGSEEPG | 60 |
| SETSDAKSTP TAEAEEAGIG DTPSLEDEAA GHVTQARMVS KSLDGTGSDD KKAKGADGKT | 120 |
| LIATPRGAAP PGQKGQANAT RIPAKTPPAP KTPPSSGEPP KSGDRSGYSS PGSPGTPGSR | 180 |
| SRTPSLPTPP TREPKKVAVV RTPPKSPSSA KSRLQTAPVP MPDLKNVKSK IGSTENLKHQ | 240 |
| PGGGKVQIIN KKLDLSNVQS KCGSLDNILH VPGGGSVQIV YKPVDLSKVT SKCGSLGNIH | 300 |
| HKPGGGQVEV KSEKLDFKDR VQSKIGSLDN ITHVPGGGNK KIETHKLTER ENAKAKTDHG | 360 |
| AEIVYKSPVV SGDTSPRHLS NVSSTGSIDM VDSPQLATLA DEVSASLAKQ GL | 412 |
| SEQ ID NO: 4 contains zero N-terminal inserts and four | |
| microtubule-binding domains (0N4R) as follows: | |
| MAEPRQEFEV MEDHAGTYGL GDRLDQGGYT MHQDQEGDTD AGLKAEEAGI GDTPSLEDEA | 60 |
| AGHVTQARMV SKSKDGTGSD DKKAKGADGK TKIATPRGAA PPGQKGQANA TRIPAKTPPA | 120 |
| PKTPPSSGEP PKSGDRSGYS SPGSPGTPGS RSRTPSLPTP PTREPKKVAV VATPPKSPSS | 180 |
| AKSRLQTAPV PMPDLKNVKS LIGSTENLKH QPGGGKVQII NKKLDLSNVQ SKCGSKDNIK | 240 |
| HVPGGGSVQI VYKPVDLSKV TSKCGSLGNI HHKPGGGQVE VKSEKLDFKD RVQSKIGSLD | 300 |
| NITHVPGGGN KKIETHKLTF RENAKALTDH GAEIVYKSPV VSGDTSPRHL SNVSSTGSID | 360 |
| MVDSPQLATL ADEVSASLAK QGL | 383 |
| SEQ ID NO: 5 contains one N-terminal insert and three | |
| microtubule-binding domains (1N3R) as follows: | |
| MAEPRQEFEV MEDHAGTYGL GDRKDQGGYT MHQDQEGDTD AGLKESPLQT PTEDGSEEPG | 60 |
| SETSDAKSTP TAEAEEAGIG DTPSLEDEAA GHVTQARMVS KSKDGTGSDD KKAKGADGKT | 120 |
| KIATPRGAAP PGQKGQANAT RIPAKTPPAP KTPPSSGEPP KSGDRSGYSS PGSPGTPGSR | 180 |
| SRTPSLPTPP TREPKKVAVV RTPPKSPSSA KSRLQTAPVP MPDLKNVKSK IGSTENLKHQ | 240 |
| PGGGKVQIVY KPVDLSKVTS KCGSLGNIHH KPGGGQVEVK SEKLDFKDRV QSKIGSLDNI | 300 |
| THVPGGGNKK IETHKLTFRE NAKAKTDHGA EIVYKSPVVS GDTSPRHLSN VSSTGSIDMV | 360 |
| DSPQLATLAD EVSASLAKQG L | 381 |
| SEQ ID NO: 6 contains zero N-terminal inserts and three | |
| microtubule-binding domains (0N3R) as follows: | |
| MAEPRQEFEV MEDHAGTYGL GDRKDQGGYT MHQDQEGDTD AGLKAEEAGI GDTPSLEDEA | 60 |
| AGHVTQARMV SKSKDGTGSD DKKAKGADGK TKIATPRGAA PPGQKGQANA TRIPAKTPPA | 120 |
| PKTPPSSGEP PKSGDRSGYS SPGSPGTPGS RSRTPSLPTP PTREPKKVAV VRTPPKSPSS | 180 |
| AKSRLQTAPV PMPDLKNVKS KIGSTENLKH QPGGGKVQIV YKPVDLSKVT SKCGSLGNIH | 240 |
| HKPGGGQVEV KSEKLDFKDR VQSKIGSLDN ITHVPGGGNK KIGTHKLTER ENAKAKTDHG | 300 |
| AEIVYKSPVV SGDTSPRHLS NVSSTGSIDM VDSPQLATLA DEVSASLAKQ GL | 352 |

TauC3 is an extremely noxious, nucleating, pre-tangle, intracellular and preferentially secreted, C-terminally truncated tau fragment ending at aspartate 421. TauC3 exists in low abundance compared to full-length Tau (FLT) (2N4R) but was shown to exert a disproportionately large pathological effect. TauC3 may contribute, e.g., to seeding and spreading of pathological tau aggregation.

Pathological aggregation of tau and spreading of pathological tau in the brain is associated with over 20 neurodegenerative disorders including, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc. (collectively referred to as “tauopathies”).

### SUMMARY OF THE INVENTION

It is an object of the invention to provide a chimeric antibody that could be used in the diagnosis and treatment of neurodegenerative disorders associated with pathological activities of TauC3 in the brain.

It is also an object of the invention to provide a humanized antibody that could be used in the diagnosis and treatment of neurodegenerative disorders associated with pathological activities of TauC3 in the brain.

It is also an additional object of the invention to provide a human antibody that could be used in the diagnosis and treatment of neurodegenerative disorders associated with pathological activities of TauC3 in the brain.

It is a further object of the invention to provide a chimeric antibody that is specific for the C-terminus of TauC3.

It is a further object of the invention to provide a chimeric antibody that is specific for the C-terminus of TauC3 and has an off-rate (K_{d}) of 1×10⁻³ s⁻¹ or less.

It is also an object of the invention to provide a humanized antibody that is specific for the C-terminus of TauC3.

It is also an object of the invention to provide a humanized antibody that is specific for the C-terminus of TauC3 and has an off-rate (K_{d}) of 1×10⁻³ s⁻¹ or less.

In furtherance of the above objects and others, the invention is directed to chimeric, humanized and human antibodies that are specific for the C-terminus of TauC3 (“anti-TauC3 antibodies”). The anti-TauC3 antibodies have a binding affinity (KD) for TauC3 of from 1×10⁻¹⁰ to 1×10⁻¹² and a binding affinity (KD) for full length tau (“FLT”) (SEQ ID NO:1) of from 1×10⁻⁴ to 1×10⁻⁸ M. For example, the anti-TauC3 antibodies may have a binding affinity (KD) for TauC3 of about 5×10⁻¹² M to about 1.2×10⁻¹⁰ M, from about 1×10⁻¹¹ M to about 1×10⁻¹⁰ M, from about 1×10⁻¹¹ M to about 9×10⁻¹¹ M, from about 1×10⁻¹¹ M to about 8×10⁻¹¹ M, from about 1×10⁻¹¹ M to about 7×10⁻¹¹ M, from about 1×10⁻¹¹ M to about 6×10⁻¹¹ M, from about 1×10⁻¹¹ M to about 5×10⁻¹¹ M, or from about 1×10⁻¹¹ M to about 4×10⁻¹¹ M; and a binding affinity (KD) for FLT of from 1×10⁻⁴ to 1×10⁻⁸ M. In the preferred embodiments, the antibody retains its binding capability after being subjected to a temperature from about 40° C. to about 67° C. for 10 minutes and also retains its binding capability after incubation in serum (e.g., mouse) at 37° C. for 21 days. The high capability of the anti-TauC3 antibodies allows the antibodies to target TauC3 without compromising normal physiological functions of FLT. In some embodiments, the specificity of the antibody allows to target only the most noxious species of tau. This may allow one, e.g., to potentially reduce the effective therapeutic dose, as compared to an antibody which is not specific and does not discriminate between different species of tau. The anti-TauC3 antibodies, and their antigen binding fragments, could be used, e.g., in the diagnosis and treatment of neurodegenerative disorders associated with pathological activities of TauC3 in the brain, including, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc. The anti-TauC3 antibody may have an aqueous solubility of 50 mg/ml or more (e.g., from about 50 mg/ml to about 200 mg/ml, from about 55 mg/ml to about 180 mg/ml, from about 55 mg/ml to about 170 mg/ml, from about 55 mg/ml to about 150 mg/ml, from about 55 mg/ml to about 140 mg/ml, from about 55 mg/ml to about 130 mg/ml; or from about 60 mg/ml to about 130 mg/ml.

The invention is further directed to chimeric, humanized and human anti-TauC3 antibodies that have a higher binding affinity (KD) for TauC3 than a murine anti-TauC3 antibody. In some embodiments, the chimeric, humanized and human anti-TauC3 antibodies have a binding affinity (KD) for TauC3 that is at least 2-fold higher, 3-fold higher or 4-fold higher than the binding affinity (KD) for TauC3 of the murine anti-TauC3 antibody. In some embodiments, the murine anti-TauC3 antibody has a binding affinity KD for TauC3 of about 4.9×10⁻¹¹ M, and the chimeric, humanized and human anti-TauC3 antibody has a binding affinity KD for TauC3 of from about 1×10⁻¹¹M to about 2.5×10⁻¹¹ M. The chimeric, humanized and human anti-TauC3 antibody may have a binding affinity KD for TauC3 of, e.g., about 1.1×10⁻¹¹ M, about 1.3×10⁻¹¹ M, about 1.5×10⁻¹¹ M, about 1.7×10⁻¹¹ M, about 1.9×10⁻¹¹ M, about 2.1×10⁻¹¹ M, or about 2.3×10⁻¹¹ M. The anti-TauC3 antibody may have an aqueous solubility of 50 mg/ml or more (e.g., from about 50 mg/ml to about 200 mg/ml, from about 55 mg/ml to about 180 mg/ml, from about 55 mg/ml to about 170 mg/ml, from about 55 mg/ml to about 150 mg/ml, from about 55 mg/ml to about 140 mg/ml, from about 55 mg/ml to about 130 mg/ml; or from about 100 mg/ml to about 200 mg/ml, from about 100 mg/ml to about 180 mg/ml, from about 100 mg/ml to about 170 mg/ml, from about 100 mg/ml to about 150 mg/ml, from about 100 mg/ml to about 140 mg/ml, or from about 100 mg/ml to about 130 mg/ml).

The invention is also directed to chimeric, humanized and human anti-TauC3 antibodies that have a binding affinity (KD) for TauC3 of from 1×10⁻¹⁰ to 1×10⁻¹², with an off-rate (K_{d}) of 1×10⁻³ s⁻¹ or less, and a binding affinity (KD) for FLT of from 1×10⁻⁴ to 1×10⁻⁸ M.

An anti-TauC3 antibody, or an antigen-binding fragment thereof, comprises (a) a heavy chain variable region comprising CDR1 represented by sequence GFTFNTYA (SEQ ID NO: 7), CDR2 represented by IRSKSNNYAT (SEQ ID NO: 8), and CDR3 represented by VGGGDF (SEQ ID NO: 9); and (b) a light chain variable region comprising CDR1 represented by sequence QEISVY (SEQ ID NO: 10), CDR2 represented by sequence GAF (SEQ ID NO: 11), and CDR3 represented by sequence LQYVRYPWT (SEQ ID NO: 12); and has a binding affinity (KD) for TauC3 of from 1×10⁻¹⁰ and 1×10⁻¹² and an off-rate (K_{d}) of 1×10⁻³ or less, and a binding affinity (KD) for FLT (SEQ ID NO:1) of from 1×10⁻⁴ to 1×10⁻⁸ M, or no detectable binding with FLT (SEQ ID NO:1).

In certain embodiments, the anti-TauC3 antibody, or an antigen-binding fragment thereof, comprises (a) a heavy chain variable region comprising CDR1 homologous to sequence GFTFNTYA (SEQ ID NO: 7), CDR2 homologous to IRSKSNNYAT (SEQ ID NO: 8), and CDR3 homologous to VGGGDF (SEQ ID NO: 9); and (b) a light chain variable region comprising CDR1 homologous to sequence QEISVY (SEQ ID NO: 10), CDR2 homologous to sequence GAF (SEQ ID NO: 11), and CDR3 homologous to sequence LQYVRYPWT (SEQ ID NO: 12); and has a binding affinity (KD) for TauC3 of from 1×10⁻¹⁰ and 1×10⁻¹² and an off-rate (K_{d}) of 1×10⁻³ or less, and a binding affinity (KD) for FLT (SEQ ID NO:1) of from 1×10⁻⁴ to 1×10⁻⁸ M, or no detectable binding with FLT (SEQ ID NO:1).

In certain embodiments, the anti-TauC3 antibody, or an antigen-binding fragment thereof, comprises (a) a heavy chain variable region comprising CDR1 identical to sequence GFTFNTYA (SEQ ID NO: 7), CDR2 identical to IRSKSNNYAT (SEQ ID NO: 8), and CDR3 identical to VGGGDF (SEQ ID NO: 9); and (b) a light chain variable region comprising CDR1 identical to sequence QEISVY (SEQ ID NO: 10), CDR2 identical to sequence GAF (SEQ ID NO: 11), and CDR3 identical to sequence LQYVRYPWT (SEQ ID NO: 12); and has a binding affinity (KD) for TauC3 of from 1×10⁻¹⁰ and 1×10⁻¹² and an off-rate (K_{d}) of 1×10⁻³ or less, and a binding affinity (KD) for FLT (SEQ ID NO:1) of from 1×10⁻⁴ to 1×10⁻⁸ M, or no detectable binding with FLT (SEQ ID NO:1).

In certain embodiments, an anti-TauC3 antibody, or an antigen-binding fragment thereof, comprises (a) a heavy chain variable region comprising CDR1 of sequence GFTFNTYA (SEQ ID NO: 7), CDR2 of sequence IRSKSNNYAT (SEQ ID NO: 8), and CDR3 of sequence VGGGDF (SEQ ID NO: 9); and (b) a light chain variable region comprising CDR1 of sequence QEISVY (SEQ ID NO: 10), CDR2 of sequence GAF (SEQ ID NO: 11), and CDR3 of sequence LQYVRYPWT (SEQ ID NO: 12; and has a binding affinity (KD) for TauC3 of from 1×10⁻¹⁰ and 1×10⁻¹² and an off-rate (K_{d}) of 1×10⁻³ or less, and a binding affinity (KD) for SEQ ID NO:1 of from 1×10⁻⁴ to 1×10⁻⁸ M, or no detectable binding with SEQ ID NO:1, and is used for treating Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD). The antibody may also be used to diagnose a tauopathy, e.g., Alzheimer disease (AD), progressive supranuclear palsy (PSP), frontotemporal dementia (FTD), traumatic brain injury (TBI), Pick's disease (PiD), corticobasal degeneration (CBD), or frontotemporal lobar degeneration (FTLD).

In one aspect, the invention is directed to an anti-TauC3 antibody, which is a humanized antibody comprising (a) a heavy chain variable region comprising CDR1 represented by SEQ ID NO: 7, CDR2 represented by SEQ ID NO: 8, and CDR3 represented by SEQ ID NO: 9; and (b) a light chain variable region comprising CDR1 represented by SEQ ID NO: 10, CDR2 represented by SEQ ID NO: 11, and CDR3 represented by SEQ ID NO: 12; and has a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT. Thus, the humanized antibody may comprise (a) a heavy chain variable region comprising CDR1 of SEQ ID NO: 7, CDR2 of SEQ ID NO: 8, and CDR3 of SEQ ID NO: 9; and (b) a light chain variable region comprising CDR1 of SEQ ID NO: 10, CDR2 of SEQ ID NO: 11, and CDR3 of SEQ ID NO: 12; and has a binding affinity (KD) for TauC3 of from 1×10⁻¹⁰ and 1×10⁻¹² and an off-rate (K_{d}) of 1×10⁻³ or less (e.g., from 1×10⁻⁴ to 1×10⁻³ s⁻¹), and a binding affinity (KD) for SEQ ID NO:1 of from 1×10⁻⁴ to 1×10⁻⁸ M, or no detectable binding with FLT, and used in treating a tauopathy, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc.

In one aspect, the invention is directed to an anti-TauC3 antibody, which is a humanized antibody comprising (a) a heavy chain variable region comprising CDR1 identical to SEQ ID NO: 7, CDR2 identical to SEQ ID NO: 8, and CDR3 identical to SEQ ID NO: 9; and (b) a light chain variable region comprising CDR1 identical to SEQ ID NO: 7, CDR2 identical to SEQ ID NO: 11, and CDR3 identical to SEQ ID NO: 12; and has a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT. Thus, the humanized antibody may comprise (a) a heavy chain variable region with CDR1 of SEQ ID NO: 7, CDR2 of SEQ ID NO: 8, and CDR3 of SEQ ID NO: 9; and (b) a light chain variable region with CDR1 of SEQ ID NO: 10, CDR2 of SEQ ID NO: 11, and CDR3 of SEQ ID NO: 12; and has a binding affinity (KD) for TauC3 of from 1×10⁻¹⁰ and 1×10⁻¹² and an off-rate (K_{d}) of 1×10⁻³ or less (e.g., from 1×10⁻⁴ to 1×10⁻³ s⁻¹), and a binding affinity (KD) for SEQ ID NO:1 of from 1×10⁻⁴ to 1×10⁻⁸ M, or no detectable binding with FLT, and used in treating a tauopathy, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc.

In one aspect, the invention is directed to an anti-TauC3 antibody, which is a humanized antibody comprising (a) a heavy chain variable region comprising CDR1 homologous to SEQ ID NO: 7, CDR2 homologous to SEQ ID NO: 8, and CDR3 homologous to SEQ ID NO: 9; and (b) a light chain variable region comprising CDR1 homologous to SEQ ID NO: CDR2 homologous to SEQ ID NO: 11, and CDR3 homologous to SEQ ID NO: 12; and has a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT. Thus, the humanized antibody may comprise (a) a heavy chain variable region comprising CDR1 of SEQ ID NO: 7, CDR2 of SEQ ID NO: 8, and CDR3 of SEQ ID NO: 9; and (b) a light chain variable region comprising CDR1 of SEQ ID NO: 10, CDR2 of SEQ ID NO: 11, and CDR3 of SEQ ID NO: 12; and has a binding affinity (KD) for TauC3 of from 1×10⁻¹⁰ and 1×10⁻² and an off-rate (K_{d}) of 1×10⁻³ or less (e.g., from 1×10⁻⁴ to 1×10⁻³ s⁻¹), and a binding affinity (KD) for SEQ ID NO:1 of from 1×10⁻⁴ to 1×10⁻⁸ M, or no detectable binding with FLT, and used in treating a tauopathy, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc.

The humanized antibody may, e.g., comprise:
(a) a variable heavy chain comprising sequence LVQLVESGGGLVQPGGSLKLSCAASGFTFNTYAMNWVRQASGKGLEWVARIRSKS-NNYATYYAASVKGRFTISRDDSKSMAYLQMDSLKTEDTAVYYCVGGGDFWGQGTLVT VSS (SEQ ID NO: 13) or a sequence homologous to SEQ ID NO: 13; and
(b) the variable light chain comprising a sequence selected from the group consisting of DIQMTQSPSSLSASVGDRVTITCRASQEISVYLGWFQQKPGKAPKRLIYGAFKLQSGVPS RFSGSRSGTEFTLTISSLQPEDFATYYCLQYVRYPWTFGGGTKVEIK (SEQ ID NO: 14) or a sequence homologous to SEQ ID NO: 14,
DIQMTQSPSSLSASVGDRVTITCRASQEISVYLGWYQQKPGKAPKRLIYGAFTLQ SGVPSRFSGSRSGTEYTLTISSLQPEDFATYYCLQYVRYPWTFGGGTKVEIK (SEQ ID NO: 15) or a sequence homologous to SEQ ID NO: 15,
DIQMTQSPSSLSASVGDRVTITCRASQEISVYLGWYQQKPGKAPKRLIYGAFSLQ SGVPSRFSGSRSGTEYTLTISSLQPEDFATYYCLQYVRYPWTFGGGTKVEIK (SEQ ID NO: 16) or a sequence homologous to SEQ ID NO: 16,
DIQMTQSPSSLSASVGDRVTITCRASQEISVYLGWFQQKPGKAPKRLIYGAFKLQ SGVPSRFSGSRSGTEYTLTISSLQPEDFATYYCLQYVRYPWTFGGGTKVEIK (SEQ ID NO: 17) or a sequence homologous to SEQ ID NO: 17, and
DIQMTQSPSSLSASVGDRVTITCRASQEISVYLSWFQQKPGKAIKRLIYGAFSLQS GVPSRFSGSRSGTEYTLTISSLQPEDFATYYCLQYVRYPWTFGGGTKVEIK (SEQ ID NO: 18) or a sequence homologous to SEQ ID NO: 18, and
have a binding affinity (KD) for TauC3 of from 1×10⁻¹⁰ and 9×10⁻¹², and a binding affinity (KD) for FLT (SEQ ID NO:1) of from 1×10⁻⁴ to 1×10⁻⁸ M, or no detectable binding with FLT.

In certain embodiments, the humanized antibody comprises a variable heavy chain (V_{H}) polypeptide of SEQ ID NO: 13 and the variable light chain (V_{L}) polypeptide of SEQ ID NO: 14, SEQ ID NO: 15, SEQ ID NO: 17, or SEQ ID NO: 18.

In certain embodiments, the humanized antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, the variable heavy chain (V_{H}) polypeptide possessing at least 70% sequence identity to SEQ ID NO: 13; and (b) a variable light chain (V_{L}) polypeptide comprising CDR1 represented by SEQ ID NO: 10, CDR2 represented by SEQ ID NO: 11, and CDR3 represented by SEQ ID NO: 12, the variable light chain (V_{L}) polypeptide possessing at least 70% sequence identity to SEQ ID NO: 14, SEQ ID NO: 15, SEQ ID NO: 16, SEQ ID NO: 17, or SEQ ID NO: SEQ ID NO: 18.

In certain embodiments, the humanized antibody comprises a V_{H} chain polypeptide possessing at least 75% sequence identity to SEQ ID NO: 13, and a V_{L} chain polypeptide possessing at least 75% sequence identity to SEQ ID NO: 14, SEQ ID NO: 15, SEQ ID NO: 16, SEQ ID NO: 17, or SEQ ID NO: 18.

In certain embodiments, the humanized antibody comprises a V_{H} chain polypeptide possessing at least 80% sequence identity to SEQ ID NO: 13, and a V_{L} chain polypeptide possessing at least 80% sequence identity to SEQ ID NO: 14, SEQ ID NO: 15, SEQ ID NO: 16, SEQ ID NO: 17, or SEQ ID NO: 18.

In certain embodiments, the humanized antibody comprises a V_{H} chain polypeptide possessing at least 85% sequence identity to SEQ ID NO: 13, and a V_{L} chain polypeptide possessing at least 85% sequence identity to SEQ ID NO: 14, SEQ ID NO: 15, SEQ ID NO: 16, SEQ ID NO: 17, or SEQ ID NO: 18.

In certain embodiments, the humanized antibody comprises a V_{H} chain polypeptide possessing at least 90% sequence identity to SEQ ID NO: 13, and a V_{L} chain polypeptide possessing at least 90% sequence identity to SEQ ID NO: 14, SEQ ID NO: 15, SEQ ID NO: 16, SEQ ID NO: 17, or SEQ ID NO: 18.

In certain embodiments, the humanized antibody comprises a V_{H} chain polypeptide possessing at least 95% sequence identity to SEQ ID NO: 13, and a V_{L} chain polypeptide possessing at least 95% sequence identity to SEQ ID NO: 14, SEQ ID NO: 15, SEQ ID NO: 16, SEQ ID NO: 17, or SEQ ID NO: 18.

In certain embodiments, the anti-TauC3 antibody comprises a variable heavy chain (V_{H}) polypeptide comprising SEQ ID NO: 13; and the variable light chain (V_{L}) polypeptide comprising a sequence selected from the group consisting of SEQ ID NO: 14, SEQ ID NO: 15, SEQ ID NO: 16, SEQ ID NO: 17, and SEQ ID NO: 18.

In certain embodiments, the anti-TauC3 antibody comprises (i) a variable heavy chain (V_{H}) polypeptide comprising CDR1, CDR2 and CDR 3, wherein CDR1 is a polypeptide of SEQ ID NO: 7, CDR2 is a polypeptide of SEQ ID NO: 8, and CDR3 is a polypeptide of SEQ ID NO: 9; and (ii) a light chain (V_{L}) polypeptide comprising CDR1, CDR2 and CDR3, wherein CDR1 is a polypeptide of SEQ ID NO: 10, CDR2 is a polypeptide of SEQ ID NO: 11, and CDR3 is a polypeptide of SEQ ID NO: 12.

In certain embodiments, anti-TauC3 antibody comprises (i) a variable heavy chain (V_{H}) polypeptide; and (ii) a light chain (V_{L}) polypeptide, wherein the variable heavy chain (V_{H}) polypeptide is a polypeptide of SEQ ID NO: 13 and the variable light chain (V_{L}) polypeptide is a polypeptide of SEQ ID NO: 14, SEQ ID NO: 15, SEQ ID NO: 16, SEQ ID NO: 17, or SEQ ID NO: 18.

The anti-TauC3 antibody could also be a chimeric antibody comprising (a) a heavy chain variable region comprising CDR1 represented by SEQ ID NO: 7 or a sequence homologous to SEQ ID NO: 7, CDR2 represented by SEQ ID NO: 8 or a sequence homologous to SEQ ID NO: 8, and CDR3 represented by SEQ ID NO: 9 or a sequence homologous to SEQ ID NO:9; and (b) a light chain variable region comprising CDR1 represented by SEQ ID NO: 10 or a sequence homologous to SEQ ID NO: 10, CDR2 represented by SEQ ID NO: 11 or a sequence homologous to SEQ ID NO: 11, and CDR3 represented by SEQ ID NO: 12 or a sequence homologous to SEQ ID NO: 12; and having a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT (SEQ ID NO:1).

The invention is also directed to an antigen-binding fragment of an antibody comprising (a) a heavy chain variable region comprising CDR1 represented by SEQ ID NO: 7, CDR2 represented by SEQ ID NO: 8, and CDR3 represented by SEQ ID NO: 9; and (b) a light chain variable region comprising CDR1 represented by SEQ ID NO: 10, CDR2 represented by SEQ ID NO: 11, and CDR3 represented by SEQ ID NO: 12; and having a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT (SEQ ID NO:1). The antigen binding fragment of the antibody could, e.g., be a Fab fragment, a Fab′ fragment, a F(ab′)2 fragment, or a scFv fragment.

The invention is also directed to an antigen-binding fragment of an antibody comprising (a) a heavy chain variable region comprising CDR1 homologous to SEQ ID NO: 7, CDR2 homologous to SEQ ID NO: 8, and CDR3 homologous to SEQ ID NO: 9; and (b) a light chain variable region comprising CDR1 homologous to SEQ ID NO: 10, CDR2 homologous to SEQ ID NO: 11, and CDR3 homologous to SEQ ID NO: 12; and having a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT (SEQ ID NO:1). The antigen binding fragment of the antibody could, e.g., be a Fab fragment, a Fab′ fragment, a F(ab′)2 fragment, or a scFv fragment.

The invention is also directed to an antigen-binding fragment of an antibody comprising (a) a heavy chain variable region comprising CDR1 identical to SEQ ID NO: 7, CDR2 identical to SEQ ID NO: 8, and CDR3 identical to SEQ ID NO: 9; and (b) a light chain variable region comprising CDR1 identical to SEQ ID NO: 10, CDR2 identical to SEQ ID NO: 11, and CDR3 identical to SEQ ID NO: 12; and having a binding affinity (KD) for TauC3 of from 1×10⁻¹⁰ and 1×10⁻¹² and an off-rate (K_{d}) of 1×10⁻³ s⁻¹ or less (e.g., from 1×10⁻⁴ to 1×10⁻³ s⁻¹), and a binding affinity (KD) for FLT (SEQ ID NO:1) of from 1×10⁻⁴ to 1×10⁻⁸ M, or no detectable binding with FLT (SEQ ID NO:1). The antigen binding fragment of the antibody could, e.g., be a Fab fragment, a Fab′ fragment, a F(ab′)2 fragment, or a scFv fragment.

The invention is also directed to methods of blocking uptake of pathological tau, methods of blocking pathological tau seeding activities, methods of inhibiting pathological tau aggregation, and methods of blocking spreading of pathological tau, tau fibrils and tau aggregates from one neuron to another or from one part of the brain to another. The methods comprise administering an effective amount of anti-TauC3 antibodies to a subject in need thereof. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

Once administered, the anti-TauC3 antibodies may block or slow spreading of pathological tau from one neuron to another or from one part of the brain to another by, e.g., blocking or slowing down TauC3 seeding activities, e.g., by essentially blocking or slowing down intracellular uptake of TauC3. This mechanism takes place extracellularly and does not require for the anti-TauC3 antibodies to be present inside the neurons. The anti-TauC3 antibodies are able to block or slow down spreading of TauC3 tau, fibrils comprising TauC3 and aggregates comprising TauC3 from one neuron to another and from one part of the brain to another. The aggregates may comprise a heterogeneous population of full-length tau (e.g., 2N4R), tau oligomers and/or post-translationally modified tau (truncated or hyperphosphorylated). In addition to blocking intracellular uptake of TauC3 and fibrils comprising TauC3, the anti-TauC3 antibodies may also block or slow down pathological tau aggregation inside the cells (e.g., neurons). Because the antibodies have substantially no affinity for full length tau (e.g., 2N4R), the antibodies should not interfere with normal non-pathological functions of full length tau. In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The anti-TauC3 antibodies may also slow the spreading of fibrils and aggregates comprising TauC3 by binding extracellular TauC3 and aggregates comprising TauC3 released from cells, thereby preventing entry of TauC3 and aggregates comprising TauC3 into neighboring cells and slowing spread of tau aggregation from one neuron to another and from one part of the brain to another. Therefore, the anti-TauC3 antibodies may serve as means for preventing entry of TauC3 or aggregates comprising TauC3 into a cell (e.g., a neuron). In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The anti-TauC3 antibodies may also be used to slow and/or reduce neuron-to-neuron spreading of tau aggregation. For example, the anti-TauC3 antibodies may promote the disaggregation of protein fibrils comprising TauC3, block the intracellular conversion of monomeric TauC3 into fibrils and/or aggregates comprising TauC3, and promote intracellular degradation of fibrils comprising TauC3 and/or aggregates comprising TauC3. In addition to TauC3, the fibrils and aggregates may comprise a heterogeneous population of full-length tau (e.g., 2N4R), tau oligomers and/or post-translationally modified tau (truncated or hyperphosphorylated). In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The anti-TauC3 antibodies may decrease brain atrophy in a subject with a tauopathy. In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The anti-TauC3 antibodies may also inhibit formation of insoluble aggregates comprising a heterogeneous population of full-length tau (e.g., 2N4R), tau oligomers and/or post-translationally modified tau (truncated or hyperphosphorylated), e.g., reducing the amount of pathological tau in the brain (e.g., TauC3, fibrils comprising TauC3 and aggregates comprising TauC3). In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

In certain embodiments, the anti-TauC3 antibodies inhibit pathological aggregation of full-length Tau (e.g., 2N4R). In some of embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

In certain embodiments, the administration of the anti-TauC3 antibodies may immunize the subject from developing a tauopathy. In some of embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The administration of the anti-TauC3 antibodies may reduce symptom(s) of a tauopathy in a subject and/or slow down the progress of tauopathy in the subject. For example, in certain embodiments, the administration of the anti-TauC3 antibodies may improve cognitive function of and/or motor/sensorimotor function in a subject with a tauopathy. In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The anti-TauC3 antibodies of the invention may be used to treat a tauopathy in a human subject. Administrations of the anti-TauC3 antibodies for the treatments of Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD) are specifically contemplated. In some embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

In certain embodiments, the invention is directed to a method of reducing the spread of tau aggregation in the brain of a subject comprises administering a therapeutically effective amount of anti-TauC3 antibody to the subject, wherein the antibody binds TauC3, but not full length Tau. In some embodiments, the anti-TauC3 antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is further directed to a method of treating a tauopathy in a subject comprising administering a therapeutically effective amount of anti-TauC3 antibody sufficient to block TauC3 seeding activity to the subject, wherein the anti-TauC3 antibody is a humanized antibody. In some embodiments, the humanized anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating a tauopathy in a subject comprising administering a therapeutically effective amount of anti-TauC3 antibody sufficient to block reuptake of TauC3 by neurons to the subject, wherein the anti-TauC3 antibody is a chimeric antibody. In some embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating Alzheimer disease in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating progressive supranuclear palsy (PSP) in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT (SEQ ID NO:1) of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating frontotemporal dementia (FTD) in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT (SEQ ID NO:1) of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO 11, and CDR3 represented by SEQ ID NO: 12.

The invention is also directed to a method of treating traumatic brain injury (TBI) in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT (SEQ ID NO:1) of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating Pick's disease (PiD) in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT (SEQ ID NO:1) of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating corticobasal degeneration (CBD) in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT (SEQ ID NO:1) of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to a method of treating frontotemporal lobar degeneration (FTLD) in a subject comprising administering a therapeutically effective amount of anti-TauC3 antibody to the subject, wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO: 11, and CDR3 represented by SEQ ID NO: 12.

The invention is also directed to therapeutic agents and compositions for blocking intracellular uptake of pathological tau; therapeutic agents and compositions for blocking tau seeding activities; therapeutic agents and compositions for blocking tau aggregation; and therapeutic agents and compositions for blocking pathological spreading of tau, tau fibrils, tau aggregates, and fragments of any of the foregoing, from one part of the brain to another, the pathological spreading induced or modulated by TauC3. The therapeutic agents and compositions comprise the anti-TauC3 antibodies described above and below. In addition to the anti-TauC3 antibodies, the compositions of the invention may comprise one or more pharmaceutically acceptable excipient(s). The therapeutic agents or compositions may also be used for passive immunization from and treatment of tauopathies, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc. In certain embodiments, the composition may further include agent(s) that prevents TauC3 production (e.g., caspase inhibitors) or promotes clearance (e.g., small molecule TauC3 aggregation inhibitor(s)).

The invention is further directed to compositions comprising an anti-TauC3 antibody and one or more pharmaceutically acceptable excipient(s), wherein the anti-TauC3 antibody is a humanized or chimeric antibody having a binding affinity (KD) for TauC3 of from 1×10⁻¹⁰ and 1×10⁻¹², with an off-rate (K_{d}) of 1×10⁻³ s⁻¹ or less, and a binding affinity (KD) for FLT (SEQ ID NO:1) of from 1×10⁻⁴ to 1×10⁻⁸ M. In some of these embodiments, the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by SEQ ID NO: 10, CDR2 represented by SEQ ID NO 11, and CDR3 represented by SEQ ID NO: 12. The composition may, e.g., be a liquid composition. The composition comprises an effective amount of the anti-TauC3 antibody to treat a tauopathy, including, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc. In certain preferred embodiments, the compositions are stable (i.e., at least 90% of the anti-TauC3 antibodies in the composition retain their binding capability after storage of the composition at 37° C. for 21 days).

### BRIEF DESCRIPTION OF THE DRAWINGS

FIG. 1. Protein (SEQ ID NO: 122) and DNA (SEQ ID NO: 142) sequence of MoTau01 Kappa Light Chain Variable Region.

FIG. 2. Protein (SEQ ID NO: 105) and DNA (SEQ ID NO: 143) Sequence of MoTau01 Heavy Chain Variable Region.

FIG. 3. pHuK LIC vector.

FIG. 4. pHuG4 μLIC vector.

FIG. 5. Protein (SEQ ID NO: 145) and DNA (SEQ ID NO: 144) Sequence of chimeric MoTau01 VK.

FIG. 6. Protein (SEQ ID NO: 147) and DNA (SEQ ID NO: 146) Sequence of chimeric MoTau01 VH.

FIGS. 7A and 7B. Binding test of chimeric Tau01 to TauC3 and FL Tau using binding ELISA.

FIGS. 8A to 8D. Binding test of murine and chimeric Tau01 antibodies to TauC3 and FL Tau using the Octet.

FIG. 9. Protein (SEQ ID NO: 107) and DNA (SEQ ID NO:148) Sequence of Tau01 HA.

FIG. 10. Protein (SEQ ID NO: 108) and DNA(SEQ ID NO: 149) Sequence of Tau01 HB.

FIG. 11. Protein (SEQ ID NO: 109) and DNA (SEQ ID NO: 150) Sequence of Tau01 HC.

FIG. 12. Protein (SEQ ID NO: 124) and DNA (SEQ ID NO: 151) Sequence of Tau01 KA.

FIG. 13. Protein (SEQ ID NO: 125) and DNA(SEQ ID NO: 152) Sequence of Tau01 KB.

FIG. 14. Protein (SEQ ID NO: 126) and DNA (SEQ ID NO:154) Sequence of Tau01 KC.

FIG. 15. Binding of humanized and chimeric Tau01 to TauC3: A and B versions.

FIGS. 16A and 16B. Binding ELISA of humanized and chimeric Tau01 to TauC3: HA to HL variants.

FIG. 17. Octet screening of humanized Tau01 antibodies to TauC3: HB, HC and HF with KA-KC.

FIGS. 18A and 18B. Binding ELISA of the humanized Tau01 antibodies to TauC3: KA to KJ variants.

FIGS. 19A and 19B. Octet screening of the humanized Tau01 antibodies to TauC3: HB with KA to KJ variants.

FIGS. 20A to 20C. Binding ELISA of the second round of humanized Tau01 antibodies to TauC3: HM, HN and HO variants.

FIG. 21. Octet screening of the second round of humanized Tau01 antibodies to TauC3: HM, HN and HO variants.

FIG. 22. Binding ELISA of HC and HM containing humanized antibodies to TauC3.

FIG. 23. Octet screening of the second round of humanized Tau01 antibodies to TauC3: HM variants.

FIGS. 24A and 24B. Octet screening of the second round of humanized Tau01 antibodies to TauC3 and FL Tau: Lead variants.

FIGS. 25A and 25B. Off-rate ranking of the lead humanized candidates with TauC3 using Biacore.

FIGS. 26A and 26B. Binding test to FL Tau by Biacore.

FIG. 27. Thermal stability of the chimeric and humanized candidate antibodies.

FIGS. 28A to 28C. SEC-MALS aggregation analysis of purified MoTau01 HuG4K and Tau01 HCKB HuG4K antibodies.

FIGS. 29A to 29D. DLS analysis of purified Tau01 HMKN, HMKO, HMKP, HMKE, and HMKM HuG4K antibodies.

FIGS. 30A to 30G. Mass spectrometry of purified chimeric and humanized candidate antibodies.

FIGS. 31A to 31G. Biacore kinetics of the humanized candidate antibodies.

FIG. 32. Thermal Shift Analysis of the humanized candidate antibodies.

FIG. 33. Non-specific protein-protein interactions (Cross-interaction chromatography) of the humanized candidate antibodies.

FIG. 34. Purified humanized antibody candidates assessed for solubility.

FIGS. 35A to 35D. Freeze/Thaw and heat stress analysis of the humanized candidate antibodies by Circular Dichroism.

FIGS. 36A to 36D. Capillary Isoelectric focusing to determine the isoeletric points of the humanized candidates.

FIGS. 37A to 37F. Serum stability assessment of the humanized candidate antibodies.

### DEFINITIONS

“Antibody” as used herein is meant to include intact molecules (i.e., a full length antibody (IgM, IgG, IgA, IgE)) and fragments thereof, as well as synthetic and biological derivatives thereof, such as for example Fab, F(ab′₂ an Fv fragments-free or expressed, e.g., on the surface of filamentous phage on pIII or pVIII or other surface proteins, or on the surface of bacteria, which are capable of binding an antigen. Fab, F(ab′₂ and Fv fragments lack the Fc fragments of intact antibody, clear more rapidly from the circulation and may have less non-specific tissue binding of antibody. The antibody may be a monoclonal antibody. Recombinant antibodies are encompassed by the term “antibody.” The term “antibody” encompasses chimeric and humanized antibodies. The antibody may also be a fully human antibody (e.g., from a transgenic mice or phage).

The term “humanized antibody” as used herein refers to an antibody in which the complementary-determining regions (CDRs) of a mouse or other non-human antibody are grafted onto a human antibody framework. By human antibody framework is meant the entire human antibody excluding the CDRs.

The term “human antibody” as used herein refers to an antibody in which the entire sequence derived from human genetic repertoire (e.g., from a transgenic mice or phage).

The term “homologous” as used herein means that that the sequence is at least 80% identical to the sequence it is homologous to, and the polymeric peptide (e.g., an antibody) comprising the homologous sequence(s) has substantially same biological activity as the polymeric peptide comprising the sequence(s) it is homologous to. For example, a humanized antibody comprising (a) a heavy chain variable region comprising CDR1 represented by sequence GFTFNTYA (SEQ ID NO: 7), CDR2 represented by IRSKSNNYAT (SEQ ID NO: 8), and CDR3 represented by VGGGDF (SEQ ID NO: 9); and (b) a light chain variable region comprising CDR1 represented by sequence QEISVY (SEQ ID NO: 10), CDR2 represented by sequence GAF (SEQ ID NO: 11), and CDR3 represented by sequence LQYVRYPWT (SEQ ID NO: 12); and an antibody in which one more of CDR sequences are replaced by a homologous sequence(s) both have a binding affinity (KD) for TauC3 of from 1×10⁻¹⁰ and 1×10⁻¹² and a binding affinity (KD) for FLT of from 1×10⁻⁴ to 1×10⁻⁸ M, or no detectable binding with FLT. By definition, homologous antibodies have substantially similar three dimensional shape.

As used herein, “CDR” means “complementary determining region.” CDRs may also be referred to as hypervariable regions. Unless otherwise specified, the CDR sequences disclosed herein are defined by IMGT numbering system.

As used herein, “represented by SEQ ID NO:” with reference to the CDR sequence means that the sequence of the CDR is identical or homologous to the recited SEQ ID NO.

The term “chimeric antibody” as used herein refers to an antibody in which the whole of the variable regions of a mouse or rat antibody are expressed along with human constant regions.

The term “murine anti-TauC3 antibody” as used herein refers to the “TauC3 antibody” characterized in Nicholls, S. B., S. L. DeVos, C. Commins, C. Nobuhara, R. E. Bennett, D. L. Corjuc, E. Maury, et al. 2017. “Characterization of TauC3 antibody and demonstration of its potential to block tau propagation.” PLoS ONE 12 (5): e0177914. doi:10.1371/journal.pone.0177914.

As used herein, “light chain” is the small polypeptide subunit of the antibody. A typical antibody comprises two light chains and two heavy chains.

As used herein, the “heavy chain” is the large polypeptide subunit of the antibody. The heavy chain of an antibody contains a series of immunoglobulin domains, with at least one variable domain and at least one constant domain.

The term “affinity” as used herein refers to the strength with which an antibody molecule binds its epitope. The affinity is determined by surface plasmon resonance (SPR) using Biacore kinetics.

The term “KD” as used herein refers to the equilibrium dissociation constant (KD=Kd/Ka, wherein Kd is a dissociation rate constant, and Ka is an association rate constant).

The term “immunodepletion” as used herein refers to the removal of proteins by the use of antibodies. The term “immunodepletion” is used interchangeably with the term “immunoprecipitation.” The term refers to the ability of an antibody to pull-down or immunoprecipitate (IP) the antigen of interest from a specimen (which would lead to immunodepletion).

As used herein, the terms “therapeutically effective amount” and “effective amount” means an amount of a therapeutic agent (e.g., an anti-TauC3 antibody) or composition that leads to a measurable clinical effect in a subject. The effective amount of the therapeutic agent is determined by the circumstances surrounding the case, including the compound administered, the route of administration, the status of the symptoms being treated and similar subjects and administration situation considerations among other considerations. An “effective amount” generally comprises from about 0.0001 mg/kg to about 100 mg/kg, preferably from 0.5 mg/kg to 20 mg/kg of the anti-TauC3 antibodies described herein. In certain embodiments, an amount of 1 mg/kg, 3 mg/kg, 4 mg/kg, 6 mg/kg, 8 mg/kg or 10 mg/kg is used.

The term “pathological tau” encompasses TauC3, fibrils comprising TauC3, and aggregates comprising TauC3 (e.g., a heterogeneous population comprising full-length tau, tau oligomers and/or post-translational modified tau (truncated or phosphorylated). In addition to TauC3, pathological tau may comprise heterogeneous population of full-length tau (e.g., 2N4R), tau oligomers and/or post-translationally modified tau (truncated or hyperphosphorylated).

The term “TauC3” means C-terminally truncated tau fragment ending at aspartate 421 of htau40 (SEQ ID NO: 1).

As used herein, “FLT” is an abbreviation for full length Tau (e.g., htau40 (SEQ ID NO: 1)).

The terms “treating” or “treatment” include attenuation, reversal, or improvement in at least one symptom or sign of symptoms associated with a tauopathy.

The term “seeding” refers to extracellular activities that precede intracellular aggregation of TauC3, fibrils comprising TauC3 and/or aggregates comprising TauC3 as part of a heterogeneous population of full-length tau (e.g., 2N4R), tau oligomers and/or post-translationally modified tau (truncated or hyperphosphorylated).

The term “aggregation” refers to the activities that take place intracellularly after TauC3 and/or fibrils comprising TauC3 and/or aggregates comprising TauC3 are taken up by the cell.

“ExpiCHO” is an abbreviation for Chinese Hamster Ovary (CHO High density/serum free) cells.

“A” is an abbreviation for Adenine.

“bp” is an abbreviation for base pairs.

“° C.” is an abbreviation for Centigrade.

“C” is an abbreviation for Cytosine.

“MEM” is an abbreviation for Minimal Essential Medium.

“DNA” is an abbreviation for Deoxyribonucleic acid.

“ELISA” is an abbreviation for Enzyme linked immuno-adsorbent assay.

“EC50” is an abbreviation for Concentration of antibody providing half-maximal response.

“EC80” is an abbreviation for Concentration of antibody providing 80% of maximal response.

“ECD” is an abbreviation for extracellular domain.

“g” is an abbreviation for grams.

“G” is an abbreviation for Guanine.

“HRP” is an abbreviation for Horseradish peroxidase.

“IgG” is an abbreviation for Immunoglobulin-G.

“K” is an abbreviation for G or T (IUPAC convention).

“LIC” is an abbreviation for Ligase independent cloning.

“min” is an abbreviation for minute.

“M” is an abbreviation for A or C (IUPAC convention).

“nm” is an abbreviation for nanometre.

“OD” is an abbreviation for optical density.

“PBS” is an abbreviation for Phosphate Buffered Saline.

“PCR” is an abbreviation for Polymerase chain reaction.

“R” is an abbreviation for A or G (IUPAC convention).

“RT” is an abbreviation for Room Temperature.

“s” is an abbreviation for second.

“S” is an abbreviation for C or G (IUPAC convention).

“T” is an abbreviation for Thymine.

“TBS” is an abbreviation for Tris Buffered Saline.

“UV” is an abbreviation for Ultra Violet.

“V” is an abbreviation for A or C or G (IUPAC convention).

“VCI” is an abbreviation for vernier, canonical and interface residues.

“VH” is an abbreviation for Immunoglobulin heavy chain variable region.

“VK” is an abbreviation for Immunoglobulin kappa light chain variable region

“W” is an abbreviation for A or T (IUPAC convention).

“Y” is an abbreviation for C or T (IUPAC convention).

### DETAILED DESCRIPTION

TauC3 is one of many species present in the high molecular weight species responsible for tau aggregation and seeding activity. In addition to TauC3, tau aggregates may comprise a heterogeneous population of full-length (normal tau), tau oligomers, and/or post-translationally modified tau (truncated or hyperphosphorylated). It has been shown that other neurodegenerative diseases other than sporadic AD also have increased levels of TauC3.

TauC3 is neurotoxic and may cause microtubule dysfunction. TauC3 may also be responsible for spreading of Tau fibrils from one part of the brain to another.

### Anti-TauC3 Antibodies

The anti-TauC3 antibodies of the invention recognize an aggregated, extracellular form of pathological TauC3. The anti-TauC3 antibody of the invention may be, e.g., a chimeric, humanized or a human anti-TauC3 antibody.

The anti-TauC3 antibodies show a very tight binding specificity for the target caspase-cleaved Tau protein when tested against the recombinant TauC3 protein. In certain embodiments, the anti-TauC3 antibody blocks seeding in the biosensor assay, and is effective for blocking entry into the neurons of the species responsible for inducing intracellular tau aggregation (i.e., effective for blocking TauC3 and TauC3 fibrils entry into the cells).

The anti-TauC3 antibodies generally have a sub-nanomolar specificity for TauC3 and as is at least 100 times more specific for TauC3 than for full length Tau (2N4R) (e.g., 100-fold or more specific for TauC3 than for full length Tau). For example, the anti-TauC3 antibodies may be from 150 to 5000 times more specific for TauC3 than for full length Tau (2N4R). In certain embodiments, the anti-TauC3 antibodies are from 500 to 2500 times more specific for TauC3 than for full length Tau (2N4R). In certain embodiments, the anti-TauC3 antibodies are from 750 to 2000 times more specific for TauC3 than for full length Tau (2N4R). In certain embodiments, the anti-TauC3 antibodies are from 1000 to 1500 times more specific for TauC3 than for full length Tau (2N4R). In all of these embodiments, the anti-TauC3 antibodies may have no detectable binding with full length Tau (2N4R).

In certain embodiments, the antibody of the invention is a chimeric, humanized or a human anti-TauC3 antibody that has a higher binding affinity (KD) for TauC3 than a murine anti-TauC3 antibody. In some embodiments, chimeric, humanized and human anti-TauC3 antibodies have a binding affinity (KD) for TauC3 that is at least 2-fold higher, 3-fold higher or 4-fold higher than the binding affinity (KD) for TauC3 of a murine anti-TauC3 antibody. In some embodiments, the murine anti-TauC3 antibody has a binding affinity KD for TauC3 of about 4.9×10⁻¹¹ M, and the chimeric, humanized and human anti-TauC3 antibody has a binding affinity KD for TauC3 of from about 1×10⁻¹¹M to about 2.5×10⁻¹¹ M. The chimeric, humanized and human anti-TauC3 antibody of the invention may have a binding affinity KD for TauC3 of, e.g., about 1.1×10⁻¹¹ M, about 1.3×10⁻¹¹ M, about 1.5×10⁻¹¹ M, about 1.7×10⁻¹¹ M, about 1.9×10−11 M, about 2.1×10⁻¹¹ M, or about 2.3×10⁻¹¹ M. In some embodiments, the murine anti-TauC3 antibody has a binding affinity KD for TauC3 of about 3.9×10⁻¹¹ M, and the chimeric, humanized and human anti-TauC3 antibody has a binding affinity KD for TauC3 of from about 1×10⁻¹¹ M to about 2.5×10⁻¹¹ M. The chimeric, humanized and human anti-TauC3 antibody of the invention may have a binding affinity KD for TauC3 of, e.g., about 1.1×10⁻¹¹ M, about 1.3×10⁻¹¹ M, about 1.5×10⁻¹¹ M, about 1.7×10⁻¹¹ M, about 1.9×10⁻¹¹ M, about 2.1×10⁻¹¹ M, or about 2.3×10⁻¹¹ M.

In certain embodiments, the antibodies bind TauC3 with equilibrium constant KD of from 1×10⁻¹⁰ M to 1×10⁻¹¹ M; and have an equilibrium constant KD with 2N4R which is from 1×10⁻⁴ M to 1×10⁻⁸ M or show no detectible binding with full length Tau (e.g., 2N4R). In the preferred embodiments, the anti-TauC3 antibodies bind TauC3 with an equilibrium constant KD of from 1×10⁻⁹ M to 1×10⁻¹² M, and bind full length (e.g., 2N4R) with an equilibrium constant KD of from 1×10⁻⁸ M to 9×10⁻⁸ M or show no detectable binding with 2N4R. In some of these embodiments, the antibodies have a very slow off rate from TauC3 (i.e., an off-rate (Kd) of from 1×10⁻⁴ to 1×10⁻³ s⁻¹) and substantially no affinity to 2N4R (i.e., ka of less than 100,000 1/MS).

In certain embodiments, the anti-TauC3 antibody is a chimeric or humanized antibody that has a KD for TauC3 of from about 5 pM to about 90 pM, from about 10 pM to about 90 pM, from about 10 pM to about 80 pM, from about 10 pM to about 70 pM, from about 10 pM to about 60 pM, from about 10 pM to about 50 pM, from about 10 to about 40 pM, or from about 10 pM to about 35 pM.

In certain embodiments, the anti-TauC3 antibody is a chimeric or humanized antibody that has a KD of from about 10 to about 90 pM and a very slow off rate from TauC3, as indicated by Kd of less than 2×10⁻³ s⁻¹. In other words, the antibodies have a high degree of specificity to TauC3, the target protein that would be produced in the diseased state, and a slow off rate, both of which are ideal for an antibody to be used in an immunization strategy.

The anti-TauC3 antibodies include but are not limited to monoclonal, chimeric, humanized, single chain, Fab fragments and a Fab expression library. The anti-TauC3 antibodies may be native or recombinant, immobilized, free in solution or displayed on the surface of various molecules or bacteria, viruses, or other surfaces.

In certain embodiments, the anti-TauC3 antibodies recognize sequence SSTGSIDMVD (SEQ ID. No. 23) at the C-terminus of TauC3, but do not recognize the same sequence when it is present internally in FLT.

Anti-TauC3 antibodies useful in accordance with the present invention (e.g. humanized antibodies) may be administered to a subject who may be susceptible to or who is suffering from a tauopathy in order to block seeding and/or aggregation of TauC3 and therefore treat one more symptoms of a tauopathy.

In yet another embodiment of the invention, the anti-TauC3 antibodies of the present invention may be conjugated to a cytoprotective agent or an agent which will facilitate and/or improve antibody's ability to cross the blood-brain barrier (“BBB”). The cytoprotective agent may be an antioxidant (e.g., melatonin); and the agent which facilitates or improves antibody's ability to cross the BBB is a hydrophobic substance which is capable of crossing the BBB, and is generally recognized as sage (GRAS) by the United States Food and Drug Administration (“FDA”). The cytoprotective agent or the agent which facilitates or improves antibody's ability to cross the BBB may be conjugated to the antibody directly or through a linker. The linker may be selected from the group comprising or consisting of a hydrazine linker, a disulfite linker, a thioether linker, a peptide linker. In certain embodiments, the antibody is has equilibrium constant KD to TauC3 that is 2-3 orders of magnitude higher than the antibodies equilibrium constant KD to 2N4R, and the cytoprotective agent is melatonin.

### Method of Use

In an aspect, the present invention provides anti-TauC3 antibodies for use in a living human with a tauopathy or at risk of developing a tauopathy. Tauopathies include, e.g., Alzheimer disease (AD), progressive supranuclear palsy (PSP), frontotemporal dementia (FTD), traumatic brain injury (TBI), Pick's disease (PiD), corticobasal degeneration (CBD), frontotemporal lobar degeneration (FTLD), etc.

### Methods of Blocking Propagation of Pathological Tau Aggregation

In one aspect, the invention is directed to a method of blocking spreading of pathological tau from one neuron to another or from one part of the brain to another.

In one aspect, the invention is directed to a method of blocking TauC3 seeding activity in the brain of a subject.

In an additional aspect, the invention is directed to a method of reducing the spread of pathological Tau aggregation in the brain of a subject.

The invention is further directed to a method of reducing the spread of aggregates comprising TauC3 in the brain of a subject.

The invention is further directed to a method of reducing the spread of fibrils comprising TauC3 in the brain of a subject.

In a further aspect the invention, the invention is directed to a method of reducing intracellular aggregation of tau induced by intracellular uptake of TauC3 and TauC3 fibrils.

In each aspect, the method comprises administering a therapeutically effective amount of anti-TauC3 antibodies to a human. The anti-TauC3 antibodies are capable of uniquely recognizing an aggregated, extracellular form of pathological tau without binding physiological tau. In a preferred embodiment, an essential part of the epitope of the anti-TauC3 antibodies is the carboxy group forming a neoepitope at the C-terminus residue in a peptide corresponding to the last ten C-terminal residues of TauC3 (e.g., TauC3 or SEQ ID NO: 23). The anti-TauC3 antibodies have equilibrium constant KD to TauC3 that is 2-3 orders of magnitude higher than the antibodies equilibrium constant KD to 2N4R and one or more pharmaceutically acceptable excipient(s). The anti-TauC3 antibodies bind TauC3 with equilibrium constant KD of from 1×10⁻¹⁰ M to 1×10⁻¹¹ M, but have an equilibrium constant KD with full length tau (e.g, 2N4R) which is from 1×10⁻⁴ M to 1×10⁻⁸ M or show no detectible binding with full length tau (e.g., 2N4R). In the preferred embodiments, the anti-TauC3 antibodies bind TauC3 with an equilibrium constant KD of from 1×10⁻¹¹ M to 9×10⁻¹¹ M, and bind 2N4R with an equilibrium constant KD of from 1×10⁻⁸ M to 9×10⁻⁸ M or show no detectable binding with 2N4R. The anti-TauC3 antibodies preferably have a very slow off rate from TauC3 and substantially no affinity to 2N4R (i.e., ka of less than 100,000 1/MS). The antibodies may, e.g., be selected from the humanized antibody, a chimeric antibody or an immunological fragment of any of the foregoing. In the preferred embodiments, the antibody is an antibody selected from the humanized anti-TauC3 antibodies described herein.

A human may or may not be having a symptom associated with tau aggregation prior to administration of a therapeutically effective amount of the anti-TauC3 antibodies. In other words, a human may or may not be experiencing a symptom associated with tau seeding and/or aggregation. One of the ordinary skill in the art will appreciate that pathological tau seeding and aggregation likely commences prior to diagnosis or the onset of symptoms associated with tau aggregation. In some embodiments, a human is having a symptom associated with tau seeding and/or aggregation. In other embodiments, a human is not having a symptom associated with tau seeding and/or aggregation. In still other embodiments, a human has detectable tau pathology but is not having any other symptom associated with tau symptoms and/or aggregation. Reducing the spread of tau aggregation in the brain of a human by administering the therapeutic agents and pharmaceutical compositions according to the invention may reduce the development and/or progression of symptoms associated with the pathological seeding and/or aggregation of tau.

Preventing, inhibiting or slowing down spreading of pathological tau aggregation may therefore be used in the treatment of pathologies associated with generation and spread of tau aggregates. One definition of symptoms associated with tau seeding and/or aggregation refers to any symptom caused by the formation of tau aggregates being composed of, in part, tau fibrils. Exemplary disorders that have symptoms associated with tau aggregation include, but are not limited to, progressive supranuclear palsy, dementia pugilistica (chronic traumatic encephalopathy), frontotemporal dementia and parkinsonism linked to chromosome 17, Lytico-Bodig disease (Parkinson-dementia complex of Guam), tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, argyrophilic grain disease (AGD), Frontotemporal lobar degeneration, Alzheimer's Disease, and frontotemporal dementia. Methods for diagnosing these disorders are known in the art.

Exemplary symptoms associated with tau seeding or aggregation may include, e.g., impaired cognitive function, altered behavior, emotional dysregulation, seizures, and impaired nervous system structure or function. Impaired cognitive function includes but is not limited to difficulties with memory, attention, concentration, language, abstract thought, creativity, executive function, planning, and organization. Altered behavior includes but is not limited to physical or verbal aggression, impulsivity, decreased inhibition, apathy, decreased initiation, changes in personality, abuse of alcohol, tobacco or drugs, and other addiction-related behaviors. Emotional dysregulation includes but is not limited to depression, anxiety, mania, irritability, and emotional incontinence. Seizures include but are not limited to generalized tonic-clonic seizures, complex partial seizures, and non-epileptic, psychogenic seizures. Impaired nervous system structure or function includes but is not limited to hydrocephalus, Parkinsonism, sleep disorders, psychosis, impairment of balance and coordination. This includes motor impairments such as monoparesis, hemiparesis, tetraparesis, ataxia, ballismus and tremor. This also includes sensory loss or dysfunction including olfactory, tactile, gustatory, visual and auditory sensation. Furthermore, this includes autonomic nervous system impairments such as bowel and bladder dysfunction, sexual dysfunction, blood pressure and temperature dysregulation. Finally, this includes hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators. Methods for detecting and evaluating symptoms associated with tau aggregation are known in the art.

In some embodiments, a symptom associated with tau aggregation refers to dementia. Dementia is not itself a specific disease, but is an overall term that describes a wide range of symptoms associated with a decline in memory or other thinking skills severe enough to reduce a person's ability to perform everyday activities. Dementia is also a shared clinical feature of many diseases associated with tau aggregation. A skilled practitioner will be familiar with the numerous methods available to diagnose the severity of dementia. For example, several cognitive tests and screening questionnaires for dementia are known in the art, all with varying degrees of sensitivity and specificity. Non-limiting examples include the mini mental state examination (MMSE), the abbreviated mental test may score (AMTS), the modified mini mental state exam (3MS), the cognitive abilities screening instrument (CASI), the Trail-making test, the clock drawing test, the Informant Questionnaire on cognitive decline in the elderly, the General practitioner assessment of cognition, the Clinical Dementia Rating (CDR), Eight-item informant interview to differentiate aging and dementia (AD8).

In some embodiments, the severity of the symptoms of dementia are quantified using the Clinical Dementia Rating. Using the Clinical Dementia Rating, a score of 0 indicates no symptoms, a score of 0.5 indicates very mild symptoms, a score of 1 indicates mild symptoms, a score of 2 indicates moderate symptoms and a score of 3 indicates severe symptoms. Thus, any increase in a Clinical Dementia Rating score for a human indicates a worsening in cognition and an increase in dementia. Moreover, change in Clinical Dementia Rating from 0 to greater than 0, indicates the development or onset of dementia.

In some embodiments, a symptom associated with tau seeding or aggregation refers to tau pathology or a tauopathy. The term “tau pathology” or “tauopathy” refers to the pathological seeding or aggregation of tau. In some embodiments, tau pathology refers to neurofibrially tangles. In other embodiments, tau pathology refers to hyperphosphorylated tau. In still other embodiments, tau pathology refers to a high level of tau aggregates detectable in blood, plasma, serum, CSF, or ISF, anywhere from 2 to approximately 40-fold higher than that detected in individuals without disease.

### Administration

Administration of the anti-TauC3 antibodies described herein can be used as a therapy to treat or immunize from tauopathies.

The antibodies in a therapeutically effective amount preferred in pharmaceutical grade, including immunologically reactive fragments, may be administered to a human. Administration is performed using standard effective techniques, include peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes, but is not limited to, via a lumbar, intraventricular or intraparenchymal catheter or using a surgically implanted controlled release formulation.

Humans amenable to treatment include individuals at risk of disease but not showing symptoms, as well as subjects presently showing symptoms. In the case of Alzheimer's disease, virtually anyone is at risk of suffering from Alzheimer's disease. Therefore, the present methods can be administered prophylactically to the general population without the need for any assessment of the risk of the subject. Such prophylactic administration can begin at, e.g., age 50 or greater. The present methods are especially useful for individuals who do have a known genetic risk of a tauopathy (e.g., Alzheimer's disease). Such individuals include those having relatives who have experienced this disease and those whose risk is determined by analysis of genetic or biochemical markers. For example, genetic markers of risk toward Alzheimer's disease include mutations in the APP gene, particularly mutations, at position 717 and positions 670 and 671 referred to as the Hardy and Swedish mutations respectively. Other markers of risk are mutations in the presenilin genes, PS1 and PS2, and ApoE4, family history of AD, hypercholesterolemia or atherosclerosis. Individuals presently suffering from Alzheimer's disease can be recognized from characteristic dementia by the presence of risk factors described above. In addition, a number of diagnostic tests are available for identifying individuals who have AD. These include imaging, and/or measurement of CSF tau and AB42 levels. Elevated tau and decreased AB42 levels signify the presence of AD. Individuals suffering from Alzheimer's disease can also be diagnosed by Alzheimer's disease and Related Disorders Association criteria.

In asymptomatic subjects, treatment can begin at any age (e.g., 10, 20, 30, 40, 50, or 60). Usually, however, it is not necessary to begin treatment until a subject reaches 40, 50, 60, 70, 75 or 80. Treatment typically entails multiple dosages over a period of time. Treatment can be monitored by assaying antibody, or activated T-cell or B-cell responses to the therapeutic agent over time. If the response falls, a booster dosage is indicated. In the case of potential Down's syndrome subjects, treatment can begin antenatally by administering therapeutic agent to the mother or shortly after birth.

In prophylactic applications, pharmaceutical compositions or medicaments are administered to a subject susceptible to, or otherwise at risk of a tauopathy in an amount sufficient to eliminate or reduce the risk, lessen the severity, or delay the outset of the disease, including biochemical, histologic and/or behavioral symptoms of the disease, its complications and intermediate pathological phenotypes presented during development of the disease. In therapeutic applications, compositions or medicaments are administered to a subject suspected of, or already suffering from, such a disease in an amount sufficient to cure, or at least partially arrest, the symptoms of the disease biochemical, histologic and/or behavioral), including its complications and intermediate pathological phenotypes in development of the disease. In some methods, administration of agent reduces or eliminates mild cognitive impairment. An amount adequate to accomplish therapeutic or prophylactic treatment is defined as a therapeutically- or prophylactically-effective dose or amount. In both prophylactic and therapeutic regimes, agents are usually administered in several dosages until a sufficient immune response has been achieved. Typically, the immune response is monitored and repeated dosages are given if the immune response starts to wane.

Effective doses of the compositions of the present invention, for the treatment of the above described conditions vary depending upon many different factors, including means of administration, target site, physiological state of the subject, other medications administered, and whether treatment is prophylactic or therapeutic. Treatment dosages need be tested on a case by case basis in clinical trials and often titrated to optimize safety and efficacy. An additional advantage of the anti-TauC3 antibodies of the present invention in certain embodiments may be that, for equal mass dosages, dosages of the anti-TauC3 of the present invention contain a higher molar dosage of the antibodies effective in clearing and/or “inactivating,” than a composition comprising antibodies that are less specific for TauC3 than the anti-TauC3 antibodies according to the present invention. Typically, anti-TauC3 antibodies of the present invention would be administered by intravenous infusion or sub cutaneous injection. The amount of the anti-TauC3 antibodies for administration by intravenous infusion may vary from 0.5 to 10 mg per subject. Subcutaneous injections generally require higher doses to reach the brain in sufficient quantity. The antibodies (i.g., whole IgG molecules) may be administered once a month.

In some methods, two or more antibodies (e.g., recombinant, monoclonal, chimeric and/or humanized) with the same or different binding specificities are administered simultaneously, in which case the dosage of each antibody administered falls within the ranges indicated. In such circumstances, the two or more antibodies may both be directed at, e.g., truncated tau. Alternatively, one or more of the antibodies may be directed at, e.g., truncated tau, and one or more additional antibodies may be directed at amyloid-P (AB) peptides associated with Alzheimer's disease. Antibodies are usually administered on multiple occasions. Intervals between single dosages can be hourly, daily, weekly, monthly, or yearly. In some methods, dosage is adjusted to achieve a plasma antibody concentration of 1-1000 μg/ml and in some methods 25-300 pg ml. Alternatively, antibody can be administered as a sustained release formulation, in which case less frequent administration is required.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the patent can be administered a prophylactic regime.

The dose of the anti-TauC3 antibodies to block TauC3 seeding is not necessarily the same as the dose of the anti-TauC3 antibodies to inhibit TauC3 aggregation. In view of the information provided in the present specification, the specific doses can be determined by routine experimentation.

The efficacy of the administration/treatment may be accessed by measuring levels of pathogenic tau or phospho tau in plasma and/or CSF. Based on this assessment, the dose and/or frequency of administration may be adjusted accordingly.

In certain embodiments, effect on cognition may also be accessed.

The efficacy may also be accessed by a degree of brain atrophy, as determined by MRI.

The safety of the administration/treatment may be accessed by number of participants experiencing adverse events (AEs), serious AEs, and abnormalities in clinical laboratory tests, vital signs, ECGs, MRI, and physical and neurological exams as well as worsening of cognition. Based on this assessment, the dose and/or frequency of administration may be adjusted accordingly.

The anti-TauC3 antibodies and immunogens may be administered intranasally, by a subcutaneous injection, intramuscular injection, IV infusion, transcutaneously, buccally, etc., or as described in more detail below.

### Pharmaceutical Compositions

Pharmaceutical compositions in accordance with the invention comprise the anti-TauC3 antibodies described herein, or fragments thereof, and one or more pharmaceutically acceptable excipients. The anti-TauC3 antibodies bind TauC3 with equilibrium constant KD of from 1×10⁻¹⁰ M to 1×10⁻¹¹ M, but have an equilibrium constant KD with full length tau (e.g, 2N4R) which is from 1×10⁻⁴ M to 1×10⁻⁸ M or show no detectible binding with full length tau (e.g., 2N4R). In the preferred embodiments, the anti-TauC3 antibodies bind TauC3 with an equilibrium constant KD of from 1×10⁻¹¹ M to 9×10⁻¹¹ M, and bind 2N4R with an equilibrium constant KD of from 1×10⁻⁸ M to 9×10⁻⁸ M or show no detectable binding with 4RTau. The anti-TauC3 antibodies preferably have a very slow off rate from TauC3 (i.e., an off-rate from 1×10⁻⁴ to 1×10⁻³ s⁻¹) and substantially no affinity to 4RTau (i.e., ka of less than 100,000 1/MS). The antibodies may, e.g., be humanized, chimeric or human (e.g., from tg mice) antibodies.

The pharmaceutical compositions are designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate.

Effective peripheral systemic delivery by intravenous or subcutaneous injection is a preferred method of administration to a living subject. Suitable vehicles for such injections are straightforward.

The concentration of humanized antibody in formulations to be administered is an effective amount and ranges from as low as about 0.1% by weight to as much as about 95 or about 99.9% by weight and will be selected primarily based on fluid volumes, viscosities, and so forth, in accordance with the particular mode of administration selected if desired. In certain embodiments, the antibodies may comprise from about 15 or about 20% by weight of the composition.

A composition for injection to a subject could be made up to contain from 1-250 ml sterile buffered water of phosphate buffered saline and about 1-5000 mg of any one of or a combination of the anti-TauC3 antibodies of the present invention. The formulation could be sterile filtered after making the formulation, or otherwise made microbiologically acceptable. A typical composition for intravenous infusion could have volumes between 1-250 ml of fluid, such as sterile Ringer's solution, and 1-100 mg per ml, or more in anti-tau antibody concentration. Therapeutic agents of the discovery can be frozen or lyophilized for storage and reconstituted in a suitable sterile carrier prior to use. Lyophilization and reconstitution may lead to varying degrees of antibody activity loss (e.g. with conventional immune globulins, IgM antibodies tend to have greater activity loss than IgG antibodies).

Dosages administered are effective amounts for the purposes indicated and may have to be adjusted to compensate. The pH of the formulations that are generally of pharmaceutical grade quality will be selected to balance antibody stability (chemical and physical) and comfort to the subject when administered. Generally, a pH between 4 and 8 is tolerated. Doses will vary from individual to individual based on size, weight, and other physiobiological characteristics of the individual receiving the successful administration.

In an aspect, a typical dose contains from about 0.1 mg to about 10 mg anti-TauC3 antibodies described herein. In certain embodiments, the typical dose contains from about 0.5 mg to about 10 mg of the anti-TauC3 antibodies. Doses can range from about 0.55 mg/kg to about 10 mg/kg. The frequency of dosing with whole IgG antibodies is usually per month whereas antibody fragments need to be dosed more often in view of their shorter half-life, as needed as to effectively treat the symptoms.

The timing of administration of the treatment relative to the disease itself and duration of treatment will be determined by the circumstances surrounding the case. Treatment could begin after diagnosis of a disease associated with tau aggregation. Alternatively, treatment could begin after clinical confirmation of a symptom associated with tau aggregation. Further still, treatment could begin after detection of tau pathology. Treatment could begin immediately in a hospital or clinic, or at a later time after discharge from the hospital or after being seen in an outpatient clinic. Duration of treatment could range from a single dose administered on a one-time basis to a life-long course of therapeutic treatments.

Although the foregoing methods appear the most convenient and most appropriate and effective for administration of proteins such as humanized antibodies, by suitable adaptation, other effective techniques for administration, such as intraventricular administration, transdermal administration and oral administration may be employed provided proper formulation is utilized herein.

Typical effective amounts or doses can be determined and optimized using standard clinical techniques and will be dependent on the mode of administration in view of the information provided herein and knowledge available in the art.

### Example 1 (Sequence Determination of Mouse MoTau01 Antibody)

### Sequence Determination of the MoTau01 Antibody

RNA Preparation from Hybridoma Cells.

Frozen pellets of mouse hybridoma cells (MoTau01), which were stored at −80° C., were supplied by Genscript on behalf of Tau-Biologic and processed using the Qiagen RNeasy Kit to isolate RNA following the manufacturer's protocol.

### 1^{st} Strand cDNA Synthesis

MoTau01 RNA (˜21 μg) was reverse-transcribed to produce cDNA using the GE Life Sciences 1^{st} strand cDNA synthesis kit following the manufacturer's protocol and purified as described in Example 5. This was repeated twice to generate 3 independent cDNA products (rounds 1, 2 and 3) in order to detect and avoid cDNA mutations induced by the Reverse Transcriptase.

### cDNA Sequence Determination

The MoTau01 cDNA was amplified by PCR as described in Example 5. Immunoglobulin cDNA was PCR-amplified with kappa light chain primers plus MKC (Table 1) or heavy chain primers (1-12 and 14) plus MHC mix (Table 2) using the Phusion Flash High-Fidelity PCR Master Mix. The MoTau01 VH PCR primer sets failed to produce any products.

Therefore, additional primers were designed based on the known sequence in the leader and terminal regions, in order to facilitate cloning the VH domain from the hybridoma cells. The additional primer sequences are included in the primer table as MHV 13 and in the ‘additional primers’ section (Table 2).

TABLE 1
PCR primers for amplifying mouse VK

| Name | Sequence (5′ → 3′) |
|---|---|
| MKV1 | TGTAAAACGACGGCCAGTATGAAGTTGCCTGTTAGGCTGTTGG |
| | TGCTG |
| | (SEQ ID NO: 20) |
| MKV2 | TGTAAAACGACGGCCAGTATGGAGWCAGACACACTCCTGYTAT |
| | GGGTG |
| | (SEQ ID NO: 21) |
| MKV3 | TGTAAAACGACGGCCAGTATGAGTGTGCTCACTCAGGTCCTGG |
| | SGTTG |
| | (SEQ ID NO: 22) |
| MKV4 | TGTAAAACGACGGCCAGTATGAGGTCCCCTGCTCAGWTTYTTG |
| | GMWTCTTG |
| | (SEQ ID NO: 23) |
| MKV5 | TGTAAAACGACGGCCAGTATGGATTTWAGGTGCAGATTWTCAG |
| | CTTC |
| | (SEQ ID NO: 24) |
| MKV6 | TGTAAAACGACGGCCAGTATGAGGTKCKKTGKTSAGSTSCTGR |
| | GG |
| | (SEQ ID NO: 25) |
| MKV7 | TGTAAAACGACGGCCAGTATGGGCWTCAAGATGGAGTCACAKW |
| | YYCWGG |
| | (SEQ ID NO: 26) |
| MKV8 | TGTAAAACGACGGCCAGTATGTGGGGAYCTKTTTYCMMTTTTT |
| | CAATTG |
| | (SEQ ID NO: 27) |
| MKV9 | TGTAAAACGACGGCCAGTATGGTRTCCWCASCTCAGTTCCTTG |
| | (SEQ ID NO: 28) |
| MKV10 | TGTAAAACGACGGCCAGTATGTATATATGTTTGTTGTCTATTT |
| | CT |
| | (SEQ ID NO: 29) |
| MKV11 | TGTAAAACGACGGCCAGTATGGAAGCCCCAGCTCAGCTTCTCT |
| | TCC |
| | (SEQ ID NO: 30) |
| CL12A | TGTAAAACGACGGCCAGTATGRAGTYWCAGACCCAGGTCTTYR |
| | T |
| | (SEQ ID NO: 31) |
| CL12B | TGTAAAACGACGGCCAGTATGGAGACACATTCTCAGGTCTTTG |
| | T |
| | (SEQ ID NO: 32) |
| CL13 | TGTAAAACGACGGCCAGTATGGATTCACAGGCCCAGGTTCTTA |
| | T |
| | (SEQ ID NO: 33) |
| CL14 | TGTAAAACGACGGCCAGTATGATGAGTCCTGCCCAGTTCCTGT |
| | T |
| | (SEQ ID NO: 34) |
| CL15 | TGTAAAACGACGGCCAGTATGAATTTGCCTGTTCATCTCTTGG |
| | TGCT |
| | (SEQ ID NO: 35) |
| CL16 | TGTAAAACGACGGCCAGTATGGATTTTCAATTGGTCCTCATCT |
| | CCTT |
| | (SEQ ID NO: 36) |
| CL17A | TGTAAAACGACGGCCAGTATGAGGTGCCTARCTSAGTTCCTGR |
| | G |
| | (SEQ ID NO: 37) |
| CL17B | TGTAAAACGACGGCCAGTATGAAGTACTCTGCTCAGTTTCTAG |
| | G |
| | (SEQ ID NO: 38) |
| CL17C | TGTAAAACGACGGCCAGTATGAGGCATTCTCTTCAATTCTTGG |
| | G |
| | (SEQ ID NO: 39) |
| MKC | CAGGAAACAGCTATGACCACTGGATGGTGGGAAGATGG |
| | (SEQ ID NO: 40) |

Ambiguity codes: W = A or T; Y = C or T; K = G or T

MKV indicates primer that hybridizes to leader sequences of the mouse kappa light chain variable region genes; MKC indicates the primer that hybridizes to the mouse kappa constant region gene. Bold underlined section indicates the M13 Forward or the M13 Reverse Sequencing Primer. Wobble bases are defined in the Definitions section.

TABLE 2
PCR primers for amplifying mouse VH

| Name | Sequence (5′ → 3′) |
|---|---|
| MHV1 | TGTAAAACGACGGCCAGTATGAAATGCAGCTGGGGCA |
| | TSTTCTTC |
| | (SEQ ID NO: 41) |
| MHV2 | TGTAAAACGACGGCCAGTATGGGATGGAGCTRTATCA |
| | TSYTCTT |
| | (SEQ ID NO: 42) |
| MHV3 | TGTAAAACGACGGCCAGTATGAAGWTGTGGTTAAACT |
| | GGGTTTTT |
| | (SEQ ID NO: 43) |
| MHV4 | TGTAAAACGACGGCCAGTATGRACTTTGGGYTCAGCT |
| | TGRTTT |
| | (SEQ ID NO: 44) |
| MHV5 | TGTAAAACGACGGCCAGTATGGACTCCAGGCTCAATT |
| | TAGTTTTCCTT |
| | (SEQ ID NO: 45) |
| MHV6 | TGTAAAACGACGGCCAGTATGGCTGTCYTRGSGCTRC |
| | TCTTCTGC |
| | (SEQ ID NO: 46) |
| MHV7 | TGTAAAACGACGGCCAGTATGGRATGGAGCKGGRTCT |
| | TTMTCTT |
| | (SEQ ID NO: 47) |
| MHV8 | TGTAAAACGACGGCCAGTATGAGAGTGCTGATTCTTT |
| | TGTG |
| | (SEQ ID NO: 48) |
| MHV9 | TGTAAAACGACGGCCAGTATGGMTTGGGTGTGGAMCT |
| | TGCTATTCCTG |
| | (SEQ ID NO: 49) |
| MHV10 | TGTAAAACGACGGCCAGTATGGGCAGACTTACATTCT |
| | CATTCCTG |
| | (SEQ ID NO: 50) |
| MHV11 | TGTAAAACGACGGCCAGTATGGATTTTGGGCTGATTT |
| | TTTTTATTG |
| | (SEQ ID NO: 51) |
| MHV12 | TGTAAAACGACGGCCAGTATGATGGTGTTAAGTCTTC |
| | TGTACCTG |
| | (SEQ ID NO: 52) |
| MHV13 | TGTAAAACGACGGCCAGTATGACATTGAACATGCTGT |
| | TGGGGC |
| | (SEQ ID NO: 140) |
| MHV14 | TGTAAAACGACGGCCAGTATGAACAGGCTTACTTCCT |
| | CATTGCTGCTGC |
| | (SEQ ID NO: 53) |
| MHCG1 | CAGGAAACAGCTATGACCCAGTGGATAGACAGATGGG |
| | GG |
| | (SEQ ID NO: 54) |
| MHCG2a | CAGGAAACAGCTATGACCCAGTGGATAGACCGATGGG |
| | GC |
| | (SEQ ID NO: 55) |
| MHCG2b | CAGGAAACAGCTATGACCCAGTGGATAGACTGATGGG |
| | GG |
| | (SEQ ID NO: 56) |
| MHCG3 | CAGGAAACAGCTATGACCCAAGGGATAGACAGATGGG |
| | GC |
| | (SEQ ID NO: 57) |

Additional Primers

| | |
|---|---|
| M13T100VHFor | TGTAAAACGACGGCCAGTGAGGTGCAGGTTGTTGAGT |
| | CTGG |
| | (SEQ ID NO: 58) |
| M13T100VHRev | TGTAAAACGACGGCCAGTGAGGTGCAGGTTGTTGAGT |
| | CTGG |
| | (SEQ ID NO: 139) |

Ambiguity codes: R = A or G; K = G or T; M = A or C.

MHV indicates primers that hybridize to the leader sequences of mouse heavy chain variable region genes. MHCG indicates primers that hybridize to the mouse constant region genes. Bold underlined section indicates the M13 Forward or the M13 Reverse Sequencing Primer. Primer MHC mix consists of an equimolar mix of primers MHCG1, MHCG2a, MHCG2b and MHCG3. ‘Wobble’ bases are defined in the Definitions section.

The result of each PCR reaction was a single amplification product that was purified using the QIAquick PCR purification kit and sequenced (by Eurofins/GATC Genomics) in both directions using the M13-Forward and M13-Reverse primers (Table 3) to obtain three independent sets of sequence information for each immunoglobulin chain.

TABLE 3
General PCR and Sequencing primers

| Name | Sequence (5′ → 3′) |
|---|---|
| HCMVi promotor | TGTTCCTTTCCATGGGTCTT |
| | (SEQ ID NO: 59) |
| HuG4_LIC_Rev | CTCTCGGAGGTGCTCCTGGAG |
| | (SEQ ID NO: 60) |
| HuK_LIC_Rev | GCAGTTCCAGATTTCAACTG |
| | (SEQ ID NO: 61) |
| M13-Forward | TGTAAAACGACGGCCAGT |
| | (SEQ ID NO: 62) |
| M13-Reverse | CAGGAAACAGCTATGACC |
| | (SEQ ID NO: 63) |

### VK and VH MoTau01 DNA Sequence

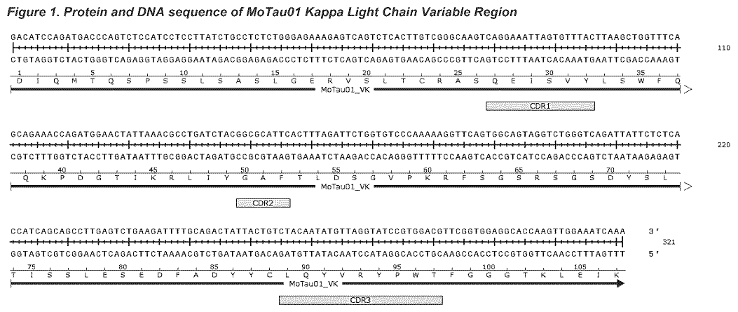

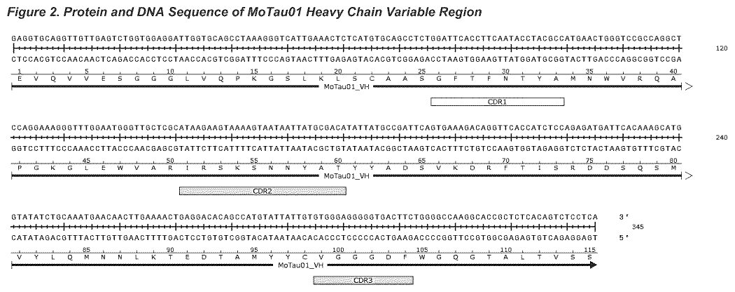

The consensus DNA sequence of the MoTau01 VK PCR product and the MoTau01 VH PCR product are shown in FIGS. 1 and 2 respectively. The variable region DNA sequences obtained were identical to the sequences determined by Genscript. Germ Line Analysis of the MoTau01 sequences show that the Kappa Light Chain is a Murine VK1 IGKV9-124*01 and the Heavy Chain is a Murine VH1 IGHV10-1*02.

### Example 2 (Generation of a Chimeric MoTau01 Antibody)

### Construction of the Chimeric MoTau01 Expression Vectors

The genes for MoTau01 VH and VK were synthesized by GenScript. Using software algorithms proprietary to GenScript, the sequences for MoTau01 VH and VK were optimized by silent mutagenesis to use codons preferentially utilized by human cells and synthesized.

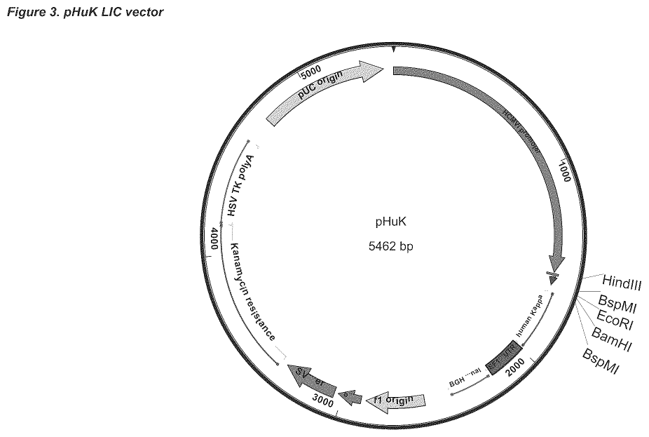

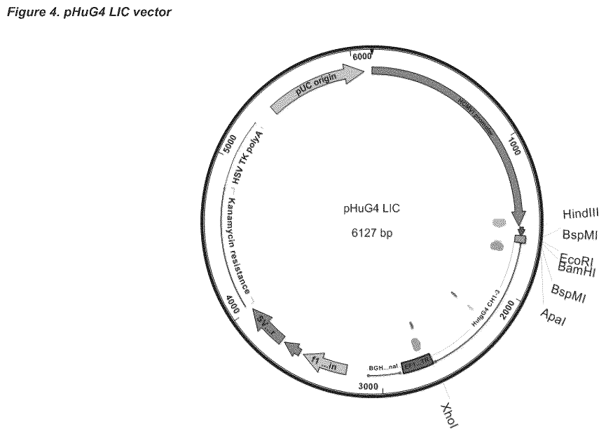

Construction of chimeric expression vectors entails cloning the synthesized variable regions into IgG/kappa vectors (pHuK and pHuG4—FIGS. 3 and 4 respectively), using ligase-independent cloning (LIC). The vectors (pCMV modified) are digested with BfuA1 (BspM1) and then compatible overhangs are generated with T4 DNA polymerase 3′-5′ exonuclease activity (+dATP).

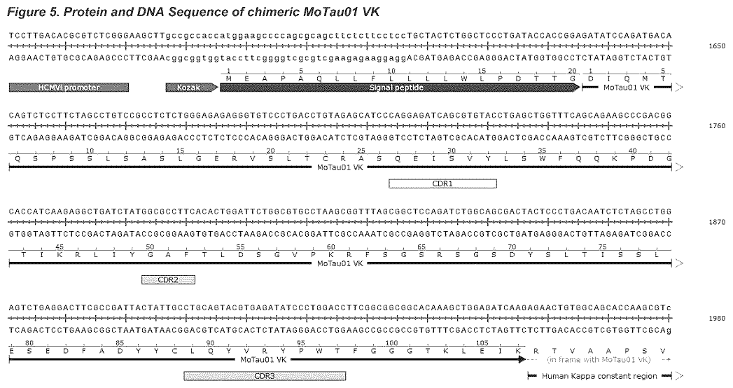

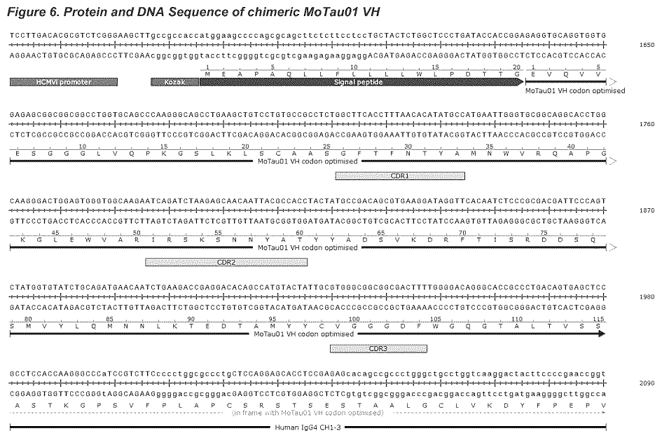

The antibody sequences (FIGS. 5 and 6) are generated by firstly amplifying the synthesized variable regions by PCR with primers containing the 3′ end of the leader sequence (most of the sequence is present in the vector)—forward primer—or the beginning of the constant region (IgG4 or kappa)—reverse primer—, followed by the beginning of the variable region (in each direction), Table 4.

TABLE 4
Cloning and mutagenesis primers

| Name | Sequence (5′ → 3′) |
|---|---|
| MoTau01 | CTCTGGCTCCCTGATACCACCGGAGAGGTGCAGGTGGTG |
| VH LIC | GAGAGC |
| For | (SEQ ID NO: 64) |
| MoTau01 | CTCTGGCTCCCTGATACCACCGGAGATATCCAGATGACA |
| VK LIC | CAGTCT |
| For | (SEQ ID NO: 65) |
| MoTau01 | GGGCCCTTGGTGGAGGCGGAGCTCACTGTCAGGGCGGT |
| VH LIC | (SEQ ID NO: 66) |
| HuG4 | |
| Rev | |
| MoTau01 | CGCTTGGTGCTGCCACAGTTCTCTTGATCTCCAGCTTTG |
| VK LIC | TGCCG |
| Huk Rev | (SEQ ID NO: 67) |
| Tau01 | CTCTGGCTCCCTGATACCACCGGACTGGTGCAGCTGGTG |
| HA/HC | GAAAGCG |
| LIC For | (SEQ ID NO: 68) |
| Tau01 | CTCTGGCTCCCTGATACCACCGGAGAGGTGCAGGTGGTG |
| HB LIC | GAAAGCG |
| For | (SEQ ID NO: 69) |
| Tau01 | GGGCCCTTGGTGGAGGCGGAGCTCACTGTCACCAGGGTG |
| HA- | (SEQ ID NO: 70) |
| HC_HuG | |
| 4 Rev | |
| Tau01 | cctgatacCACCGGAGAGGTGCAGCTGGTG |
| HD | (SEQ ID NO: 71) |
| mutation | |
| L-E For | |
| Tau01 | CACCAGCTGCACCTCTCCGGTGgtatcagg |
| HD | (SEQ ID NO: 72) |
| mutation | |
| L-E Rev | |
| Tau01 | CGGACTGGTGCAGGTGGTGGAAAGCGG |
| HE | (SEQ ID NO: 73) |
| mutation | |
| L-V For | |
| Tau01 | CCGCTTTCCACCACCTGCACCAGTCCG |
| HE | (SEQ ID NO: 74) |
| mutation | |
| L-V Rev | |
| Tau01 | TTTAACACATATGCAATGAACTGGGTGCGGCAGG |
| HF | (SEQ ID NO: 75) |
| mutation | |
| H-N For | |
| Tau01 | CCTGCCGCACCCAGTTCATTGCATATGTGTTAAA |
| HF | (SEQ ID NO: 76) |
| mutation | |
| H-N Rev | |
| Tau01 | CTGGAGTGGGTGGCCCGGATCAGATCT |
| HG | (SEQ ID NO: 77) |
| mutation | |
| G-A For | |
| Tau01 | AGATCTGATCCGGGCCACCCACTCCAG |
| HG | (SEQ ID NO: 78) |
| mutation | |
| G-A Rev | |
| Tau01 | CTAAGAGCAACAATTATGCAACATATTATGCAGCATCTG |
| HH | TGAAGGGCAG |
| mutation | (SEQ ID NO: 79) |
| A-Y For | |
| Tau01 | CTGCCCTTCACAGATGCTGCATAATATGTTGCATAATTG |
| HH | TTGCTCTTAG |
| mutation | (SEQ ID NO: 80) |
| A-Y Rev | |
| Tau01 | TATGCAACAGCATATGCAGATTCTGTGAAGGGCAGGTTC |
| HI | A |
| mutation | (SEQ ID NO: 81) |
| A-D For | |
| Tau01 | TGAACCTGCCCTTCACAGAATCTGCATATGCTGTTGCAT |
| HI | A |
| mutation | (SEQ ID NO: 82) |
| A-D Rev | |
| Tau01 | CCGCGACGATTCTAAGAGTACAGCCTATCTGCAGA |
| HJ | (SEQ ID NO: 83) |
| mutation | |
| N-S For | |
| Tau01 | TCTGCAGATAGGCTGTACTCTTAGAATCGTCGCGG |
| HJ | (SEQ ID NO: 84) |
| mutation | |
| N-S Rev | |
| Tau01 | TCTCCCGCGACGATTCTAAGAATATGGCCTATCTGCAGA |
| HK | T |
| mutation | (SEQ ID NO: 85) |
| T-M For | |
| Tau01 | ATCTGCAGATAGGCCATATTCTTAGAATCGTCGCGGGAG |
| HK | A |
| mutation | (SEQ ID NO: 86) |
| T-M Rev | |
| Tau01 | GACGATTCTAAGAATACAGTCTATCTGCAGATGGACTCC |
| HL | (SEQ ID NO: 87) |
| mutation | |
| A-V For | |
| Tau01 | GGAGTCCATCTGCAGATAGACTGTATTCTTAGAATCGTC |
| HL | (SEQ ID NO: 88) |
| mutation | |
| A-V Rev | |
| Tau01 | CTCTGGCTCCCTGATACCACCGGAGACATCCAGATGACC |
| KA/KC | CAGTCTC |
| LIC For | (SEQ ID NO: 89) |
| Tau01 | CTCTGGCTCCCTGATACCACCGGAGACATCCAGATGACA |
| LB LIC | CAGTCTC |
| For | (SEQ ID NO: 90) |
| Tau01 | CGCTTGGTGCTGCCACAGTTCTCTTGATCTCCACCTTTG |
| KA-KC | TGCCG |
| LIC Huk | (SEQ ID NO: 91) |
| Rev | |
| Tau01 | GATCTCCGTGTACCTGAGCTGGTATCAGCAGAA |
| KD | (SEQ ID NO: 141) |
| mutation | |
| G-S For | |
| Tau01 | TTCTGCTGATACCAGCTCAGGTACACGGAGATC |
| KD | (SEQ ID NO: 92) |
| mutation | |
| G-S Rev | |
| Tau01 | TGTACCTGGGCTGGTTTCAGCAGAAGCCC |
| KE | (SEQ ID NO: 93) |
| mutation | |
| Y-F For | |
| Tau01 | GGGCTTCTGCTGAAACCAGCCCAGGTACA |
| KE | (SEQ ID NO: 94) |
| mutation | |
| Y-F Rev | |
| Tau01 | GAAGCCCGGCAAGGCCATTAAGCGGCTGATCTAC |
| KF | (SEQ ID NO: 95) |
| mutation | |
| P-I For | |
| Tau01 | GTAGATCAGCCGCTTAATGGCCTTGCCGGGCTTC |
| KF | (SEQ ID NO: 96) |
| mutation | |
| P-I Rev | |
| Tau01 | ATCTACGGCGCCTTCACGCTGCAGTCCG |
| KG | (SEQ ID NO: 97) |
| mutation | |
| K-T For | |
| Tau01 | CGGACTGCAGCGTGAAGGCGCCGTAGAT |
| KG | (SEQ ID NO: 98) |
| mutation | |
| K-T Rev | |
| Tau01 | GGATCCAGATCTGGCAGCGAGTTTACCCTGA |
| KH | (SEQ ID NO: 99) |
| mutation | |
| T-S For | |
| Tau01 | TCAGGGTAAACTCGCTGCCAGATCTGGATCC |
| KH | (SEQ ID NO: 100) |
| mutation | |
| T-S Rev | |
| Tau01 | CAGATCTGGCACCGAGTATACCCTGACAATCTCTA |
| KI | (SEQ ID NO: 101) |
| mutation | |
| F-Y For | |
| Tau01 | TAGAGATTGTCAGGGTATACTCGGTGCCAGATCTG |
| KI | (SEQ ID NO: 102) |
| mutation | |
| F-Y Rev | |
| Tau01 | CTACGGCGCCTTCAGCCTGCAGTCCGGAGT |
| KJ | (SEQ ID NO: 103) |
| mutation | |
| K-S For | |
| Tau01 | ACTCCGGACTGCAGGCTGAAGGCGCCGTAG |
| KJ | (SEQ ID NO: 104) |
| mutation | |
| K-S Rev | |

The complementary overhangs were generated in the PCR products by T4 DNA polymerase+dTTP treatment (the protocol is provided in Example 5). Vector and inserts were incubated at RT, transformed into chemically-competent TOP10 bacteria and plated on Kanamycin plates. Several clones were isolated and colonies screened by PCR using primers HCMVi promotor forward and HuG4 μLIC Rev for VH or HuK LIC Rev for VK (Table 3).

The clones generating the correct sized PCR products were selected, miniprepped using the QIAGEN kit and sequenced using the same primers.

### Generation of the Chimeric Antibodies

ExpiCHO suspension cells growing in ExpiCHO transfection medium and antibiotics were co-transfected with MoTau01_VH.pHuG4 and MoTau01_VK.pHuK (1 μg DNA each) using ExpiFectamine CHO Reagent. The cells were grown in 1 mL growth medium for 7 days. Up to 160 μg/mL (Table 14 A) of MoTau01 HuG4k antibody was measured in the conditioned medium by Octet quantitation.

### TauC3 Binding Activity of Chimeric Antibodies

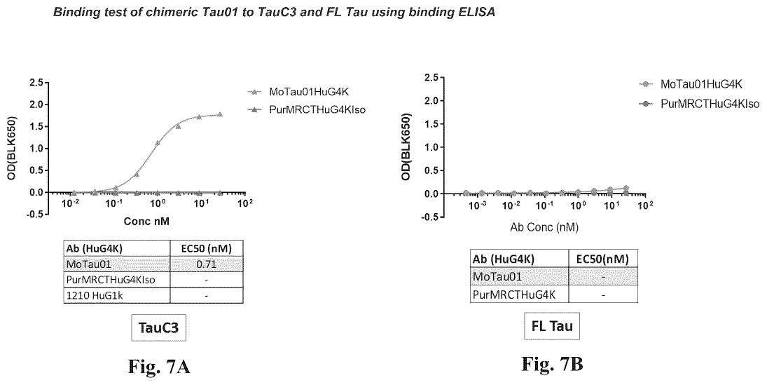

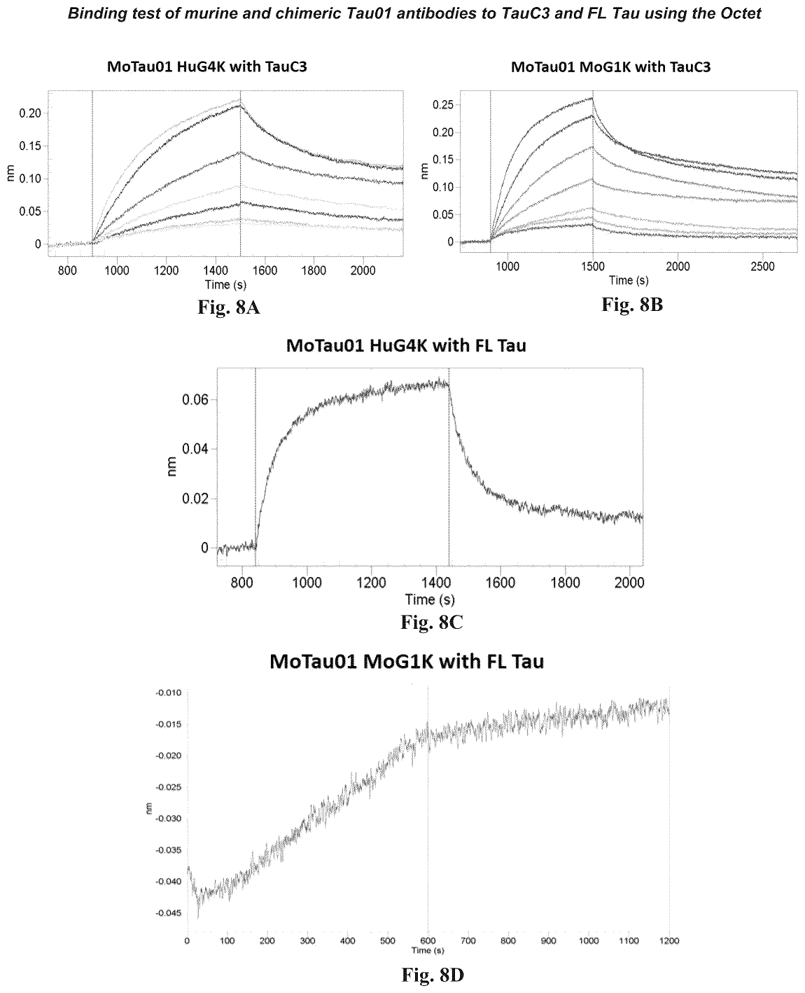

The TauC3 and FL Tau antigens were generated and purified by Genscript and supplied at a concentration of 2.54 mg/ml or 0.24 mg/ml respectively. Binding of the chimeric antibody to TauC3 and FL Tau was assayed by binding ELISA. The chimeric antibody was able to bind to TauC3 with an EC50 of 0.7 nM (FIG. 7 A) but no binding could be observed to FL Tau (FIG. 7 B). No non-specific binding to either antigen could be observed with an isotype confirming the binding observed is specific. Binding of the mouse and chimeric Tau01 antibodies to purified TauC3, was measured using Bio-Layer Interferometry (OctetRed96, ForteBio Section 8.12). The mouse and chimeric antibodies were assayed against a concentration series from 20 nM-0.31 nM TauC3 (FIGS. 8 A and B). Both the mouse and chimeric antibodies were capable of binding to TauC3 in a concentration-dependent manner. The binding of the mouse and chimeric antibodies was also tested against FL Tau but low signals or no binding was observed (FIGS. 8 C and D) confirming the binding of the antibodies is specific to TauC3.

### Example 3 Design of Tau01 Humanized Antibody Variants

### Human VH and VK cDNA Databases

The protein sequences of human and mouse immunoglobulins from the International Immunogenetics Database 2009 (Lefranc, 2015) and the Kabat Database Release 5 of Sequences of Proteins of Immunological Interest (last update 17 Nov. 1999)(Kabat et al. 1991) were used to compile a database of aligned human immunoglobulin sequences. The database contains 10,406 VH and 2,894 VK sequences.

### Molecular Model of MoTau01

The MoTau01 VH and VK sequences were used to design the humanized version of MoTau01 antibody. A homology model of MoTau01 antibody variable regions has been generated using the antibody prediction panel in Maestro 11.5. The chosen human framework was used to generate 10 loop models, which were prepared using the one-step protein preparation wizard. Protein reliability reports were generated for all 10 models and no major differences could be identified in model quality. All 10 models were used to determine a consensus of residues which were within 4 Å of the CDR loops, in order to capture different orientations of the CDRs.

### Human Framework Selection

Humanization requires the identification of suitable human V regions. The sequence analysis program, Gibbs, was used to interrogate the human VH and VK databases with MoTau01 VH and VK protein sequences using various selection criteria. Using the Maestro 11.5 (Schrödinger) software, FW residues within 4 Å of the CDR residues (IMGT definition) in the structures of mouse Tau01 antibody were identified, and designated as the “4 Å Proximity Residues”. Human VH sequence alignments with highest identity to MoTau01 VH in the 4 Å Proximity Residues are shown in Table 5. Table 6 lists these envelope residues and VCIs, and the number of residues in either the FW, VCI or 4 Å Proximity Residues which are identical to the mouse equivalent position for the sequences in Table 5.

Humanized sequences and incomplete sequences were removed from the analysis. The sequence DQ840895.1 was chosen as the human heavy chain donor candidate. This sequence scores high in sequence identity and similarity, and has only 2 somatic mutations from its IGHV3-73*01 VH germline. It has eight 4 Å proximity and one VCI residue change but this was the minimal number of changes obtainable (Table 8).

TABLE 8
Tau01 Heavy Chain Humanization Strategy

| | Sequence |
|---|---|
| | 1 2 3 4 5 |
| | 7 8 9 10 |
| | 11 |
| | 12345678901234567890123456789012345ABCD67890123456789012ABC |
| | D3 |
| | 45678901234567890123456789012ABC345678901234567890 |
| Name | ABCDEFGHIJKLMNOPQRSTUV1234567890123 |
| MoTau01 VH | EVQVVESGGGLVQPKGSLKLSCAASGFTFNT----YAMNWVRQAPGKGLEWVARIRSKS- |
| | NNYATYYADSVKDRFTISRDDSQSMVYLQMNNLKTEDTAMYYCVGGG------------- |
| | -------------DFWGQGTALTVSS (SEQ ID NO: 105) |
| DQ840895.1 | LVQLVESGGGLVQPGGSLKLSCAASGFTF---- |
| | SGSAMHWVRQASGKGLEWVGRIRSKA- |
| | NSYATAYAASVKGRFTISRDDSKNTAYLQMDSLKTEDTAVYYCTTY---- |
| | -----------------------EGWGQGTLVTVSS (SEQ ID NO: 106) |
| Tau01 HA | LVQLVESGGGLVQPGGSLKLSCAASGFTFNT---- |
| | YAMHWVRQASGKGLEWVGRIRSKS- |
| | NNYATAYAASVKGRFTISRDDSKNTAYLQMDSLKTEDTAVYYCVGGG---- |
| | ----------------------DFWGQGTLVTVSS (SEQ ID NO: 107) |
| Tau01 HB | EVQVVESGGGLVQPGGSLKLSCAASGFTFNT---- |
| | YAMNWVRQASGKGLEWVARIRSKS- |
| | NNYATYYADSVKGRFTISRDDSKSMVYLQMNDSLKTEDTAVYYCVGGG---- |
| | ----------------------DFWGQGTLVTVSS (SEQ ID NO: 108) |
| Tau01 HC | LVQLVESGGGLVQPGGSLKLSCAASGFTFNT---- |
| | YAMNWVRQASGKGLEWVGRIRSKS- |
| | NNYATYYAASVKGRFTISRDDSKSMAYLQMDSLKTEDTAVYYCVGGG---- |
| | -----------------------DFWGQGTLVTVSS (SEQ ID NO: 109) |
| Tau01 HD | EVQLVESGGGLVQPGGSLKLSCAASGFTFNT---- |
| | YAMHWVRQASGKGLEWVGRIRSKS- |
| | NNYATAYAASVKGRFTISRDDSKNTAYLQMDSLKTEDTAVYYCVGGG---- |
| | ----------------------DFWGQGTLVTVSS (SEQ ID NO: 110) |
| Tau01 HE | LVQVVESGGGLVQPGGSLKLSCAASGFTFNT---- |
| | YAMHWVRQASGKGLEWVGRIRSKS- |
| | NNYATAYAASVKGRFTISRDDSKNTAYLQMDSLKTEDTAVYYCVGGG---- |
| | ----------------------DFWGQGTLVTVSS (SEQ ID NO: 111) |
| Tau01 HF | LVQLVESGGGLVQPGGSLKLSCAASGFTFNT---- |
| | YAMNWVRWASGKGLEWVGRIRSKS- |
| | NNYATAYAASVKGRFTISRDDSKNTAYLQMDSLKTEDTAVYYCVGGG---- |
| | ----------------------DFWGQGTLVTVSS (SEQ ID NO: 112) |
| Tau01 HG | LVQLVESGGGLVQPGGSLKLSCAASGFTFNT---- |
| | YAMHWVRQASGKGLEWVARIRSKS |
| | NNYATAYAASVKGRFTISRDDSKNTAYLQMDSLKTEDTAVYYCVGGG---- |
| | ----------------------DFWGQGTLVTVSS (SEQ ID NO: 113) |
| Tau01 HH | LVQLVESGGGLVQPGGSLKLSCAASGFTFNT---- |
| | YAMHWVRQASGKGLEWVGRIRSKS- |
| | NNYATYYAASVKGRFTISRDDSKNTAYLQMDSLKTEDTAVYYCVGGG---- |
| | ----------------------DFWGQGTLVTVSS (SEQ ID NO: 114) |
| Tau01 HI | LVQLVESGGGLVQPGGSLKLSCAASGFTFNT---- |
| | YAMHWVRQASGKGLEWVGRIRSKS- |
| | NNYATAYADSVKGRFTISRDDSKNTAYLQMDSLKTEDTAVYYCVGGG---- |
| | ----------------------DFWGQGTLVTVSS (SEQ ID NO: 115) |
| Tau01 HJ | LVQLVESGGGLVQPGGSLKLSCAASGFTFNT---- |
| | YAMHWVRQASGKGLEWVGRIRSKS- |
| | NNYATAYAASVKGRFTISRDDSKSTAYLQMDSLKTEDTAVYYCVGGG---- |
| | ---------------------DFWGQGTLVTVSS (SEQ ID NO: 116) |
| Tau01 HK | LVQLVESGGGLVQPGGSLKLSCAASGFTFNT---- |
| | YAMHWVRQASGKGLEWVGRIRSKS- |
| | NNYATAYAASVKGRFTISRDDSKNMAYLQMDSLKTEDTAVYYCVGGG---- |
| | ----------------------DFWGQGTLVTVSS (SEQ ID NO: 117) |
| Tau01 HL | LVQLVESGGGLVQPGGSLKLSCAASGFTFNT---- |
| | YAMHWVRQASGKGLEWVGRIRSKS- |
| | NNYATAYAASVKGRFTISRDDSKNTVYLQMDSLKTEDTAVYYCVGGG---- |
| | ----------------------DFWGQGTLVTVSS (SEQ ID NO: 118) |
| Tau01 HM | LVQLVESGGGLVQPGGSLKLSCAASGFTFNT---- |
| | YAMNWVRQASGKGLEWVARIRSKS- |
| | NNYATYYAASVKGRFTISRDDSKSMAYLQMDSLKTEDTAVYYCVGGG---- |
| | -----------------------DFWGQGTLVTVSS (SEQ ID NO: 119) |
| Tau01 HN | LVQVVESGGGLVQPGGSLKLSCAASGFTFNT---- |
| | AYMNWVRQASGKGLEWVGRIRSKS- |
| | NNYATYYAASVKGRFTISRDDSKSMAYLQMDSLKTEDTAVYYCVGGG---- |
| | ----------------------DFWGQGTLVTVSS (SEQ ID NO: 120) |
| Tau01 HO | LVQVVESGGGLVQPGGSLKLSCAASGFTFNT---- |
| | YAMNWVRQASGKGLEWVARIRSKS- |
| | NNYATYYAASVKGRFTISRDDSKSMAYLQMDSLKTEDTAVYYCVGGG---- |
| | ----------------------DFWGQGTLVTVSS SEQ ID NO: 121) |

Residues in Italics indicate back-mutations to the Mouse Residue

Likewise, the sequence L33034 was chosen as the human kappa light chain donor candidate. This sequence scores high in sequence identity and similarity to Tau01 VK and has only 1 somatic mutation from the IGKV1-17*01 germline. It has five potential 4 Å proximity residues and one VCI residue change.

The sequences for Kappa Light Chain Humanization Strategy are shown in Table 12.

TABLE 12
Tau01 Kappa Light Chain Humanization Strategy

| | Sequence |
|---|---|
| | 1 2 3 4 5 6 |
| | 7 8 9 10 |
| | 123456789012345678901234567ABCDEF890123456789012345678901234567890123456 |
| Name | 78901234567890123456789012345ABCDEF678901234567 |
| MoTau01 VK | DIQMTQSPSSLSASLGERVSLTCRASQEIS------ |
| | VYLSWFQQKPDGTIKRLIYGAFTLDSGVPKRFSGSRSGSDYSLTISSLESEDFADYYCLQYVR----- |
| | YPWTFGGGTKLEIK (SEQ ID NO: 122) |
| L33034 | DIQMTQSPSSLSASVGDRVTITCRASQEIS------ |
| | VYLGWYQQKPGKAPKRLIYGAFKLQSGVPSRFSGSRSGTEFTLTISSLQPEDFATYYCLQYVR----- |
| | YPWTFGGGTKVEIK (SEQ ID NO: 123) |
| Tau01 KA | DIQMTQSPSSLSASVGDRVTITCRASQEIS------ |
| | VYLGWYQQKPGKAPKRLIYGAFKLQSGVPSRFSGSRSGTEFTLTISSLQPEDFATYYCLQYVR----- |
| | YPWTFGGGTKVEIK (SEQ ID NO: 124) |
| Tau01 KB | DIQMTQSPSSLSASVGDRVTITCRASQEIS------ |
| | VYLSWFQQKPGKAIKRLIYGAFTLQSGVPSRFSGSRSGSEYTLTISSLQPEDFATYYCLQYVR----- |
| | YPWTFGGGTKVEIK (SEQ ID NO: 125) |
| Tau01 KC | DIQMTQSPSSLSASVGDRVTITCRASQEIS------ |
| | VYLSWFQQKPGKAPKRLIYGAFKLQSGVPSRFSGSRSGTEYTLTISSLQPEDFATYYCLQYVR----- |
| | YPWTFGGGTKVEIK (SEQ ID NO: 126) |
| Tau01 KD | DIQMTQSPSSLSASVGDRVTITCRASQEIS------ |
| | VYLSWYQQKPGKAPKRLIYGAFKLQSGVPSRFSGSRSGTEFTLTISSLQPEDFATYYCLQYVR----- |
| | YPWTFGGGTKVEIK (SEQ ID NO: 127) |
| Tau01 KE | DIQMTQSPSSLSASVGDRVTITCRASQEIS------ |
| | VYLGWFQQKPGKAPKRLIYGAFKLQSGVPSRFSGSRSGTEFTLTISSLQPEDFATYYCLQYVR----- |
| | YPWTFGGGTKVEIK (SEQ ID NO: 128) |
| Tau01 KF | DIQMTQSPSSLSASVGDRVTITCRASQEIS------ |
| | VYLGWYQQKPGKAIKRLIYGAFKLQSGVPSRFSGSRSGTEFTLTISSLQPEDFATYYCLQYVR----- |
| | YPWTFGGGTKVEIK (SEQ ID NO: 129) |
| Tau01 KG | DIQMTQSPSSLSASVGDRVTITCRASQEIS------ |
| | VYLGWYQQKPGKAPKRLIYGAFTLQSGVPSRFSGSRSGTEFTLTISSLQPEDFATYYCLQYVR----- |
| | YPWTFGGGTKVEIK (SEQ ID NO: 130) |
| Tau01 KH | DIQMTQSPSSLSASVGDRVTITCRASQEIS------ |
| | VYLGWYQQKPGKAPKRLIYGAFKLQSGVPSRFSGSRSGSEFTLTISSLQPEDFATYYCLQYVR----- |
| | YPWTFGGGTKVEIK (SEQ ID NO: 131) |
| Tau01 KI | DIQMTQSPSSLSASVGDRVTITCRASQEIS------ |
| | VYLGWYQQKPGKAPKRLIYGAFKLQSGVPSRFSGSRSGTEYTLTISSLQPEDFATYYCLQYVR----- |
| | YPWTFGGGTKVEIK (SEQ ID NO: 132) |
| Tau01 KJ | DIQMTQSPSSLSASVGDRVTITCRASQEIS------ |
| | VYLGWYQQKPGKAPKRLIYGAFSLQSGVPSRFSGSRSGTEFTLTISSLQPEDFATYYCLQYVR----- |
| | YPWTFGGGTKVEIK (SEQ ID NO: 133) |
| Tau01 KL | DIQMTQSPSSLSASVGDRVTITCRASQEIS------ |
| | VYLSWFQQKPGKAIKRLIYGAFKLQSGVPSRFSGSRSGTEYTLTISSLQPEDFATYYCLQYVR----- |
| | YPWTFGGGTKVEIK (SEQ ID NO: 134) |
| Tau01 KM | DIQMTQSPSSLSASVGDRVTITCRASQEIS------ |
| | VYLSWFQQKPGKAIKRLIYGAFSLQSGVPSRFSGSRSGTEYTLTISSLQPEDFATYYCLQYVR----- |
| | YPWTFGGGTKVEIK (SEQ ID NO: 135) |
| Tau01 KN | DIQMTQSPSSLSASVGDRVTITCRASQEIS------ |
| | VYLGWYQQKPGKAPKRLIYGAFTLQSGVPSRFSGSRSGTEYTLTISSLQPEDFATYYCLQYVR----- |
| | YPWTFGGGTKVEIK (SEQ ID NO: 136) |
| Tau01 KO | DIQMTQSPSSLSASVGDRVTITCRASQEIS------ |
| | VYLGWYQQKPGKAPKRLIYGAFSLQSGVPSRFSGSRSGTEYTLTISSLQPEDFATYYCLQYVR----- |
| | YPWTFGGGTKVEIK (SEQ ID NO: 137) |
| Tau01 KP | DIQMTQSPSSLSASVGDRVTITCRASQEIS------ |
| | VYLGWFQQKPGKAPKRLIYGAFKLQSGVPSRFSGSRSGTEYTLTISSLQPEDFATYYCLQYVR----- |
| | YPWTFGGGTKVEIK (SEQ ID NO: 138) |

Residues in Italics indicate back-mutations to the Mouse Residue.

### Design of Tau01 Humanized Heavy Chain Variants

Once a suitable human framework has been identified, the synthetic protein and DNA sequence can be designed. The initial design of the humanized version of Tau01 is the grafting of CDR 1, 2 and 3 from MoTau01 VH into the acceptor FW of DQ840895.1, thereby creating variant Tau01 HA. The eight 4 Å proximity residues and one VCI residue, at positions 1, 4, 35, 49, 58, 61, and 76-78 are then back mutated to the mouse equivalent residue, in the humanized version Tau01 HB, and mutated one at a time in the following variants: sequences were assembled in silico and designated Tau01 HD to Tau01 HL. Table 8 compares the murine and the humanized versions of Tau01 VH protein sequences.

### Design of Tau01 Humanized Light Chain Variants

The framework from L33034 was used to design the DNA and protein for the humanized constructs. CDR 1, 2 and 3 from Tau01 VK are shown grafted into the acceptor FW of L33034 to generate the initial version of humanized Tau01. There are five unmatched 4 Å Proximity residues and one VCI residue, at positions 34, 36, 44, 53, 69 and 71, in Tau01 KA that were back-mutated to the equivalent mouse residue in the variant Tau01 KB (Table 12).

These residues were mutated one at a time in the following variants: sequences were assembled in silico and designated Tau01 KD to Tau01 KI. In version Tau01 KG, the residue K, back-mutated to the mouse residue T, was also mutated to the Human germline residue S. This additional variant was named Tau01 KJ.

### Design of the Heavy and Light Chain C Versions

Following on from the design of the initial humanized variants, a homology model of Tau01 HAKA was built and evaluated. The latter model was superimposed with the model of the mouse antibody. Each position identified for back-mutation and the 3 Å residues surrounding those positions were highlighted and examined in the models. Based on this data, predictions were made as to which residues were the most important to back-mutate and these were incorporated to form the HC version for the heavy chain (Table 8) and the KC version for the light chain (Table 12).

### Example 4 (Generation and Properties of a Humanized Antibodies)

### Generation of Tau01 Humanized Antibodies

The sequences for Tau01 HA/B/C and KA/B/C were codon optimized to use codons preferentially utilized by human cells and synthesized by Genscript. KA/B/C and HA/B/C constructs were PCR amplified and cloned into pHuK and pHuG4, respectively in ligase independent cloning reactions and used to transform TOP10 bacteria. Version HA or KA was subsequently modified by PCR mutagenesis to obtain the other humanized variants annotated in the Table 8 or 12 respectively, using the primers in Table 4.

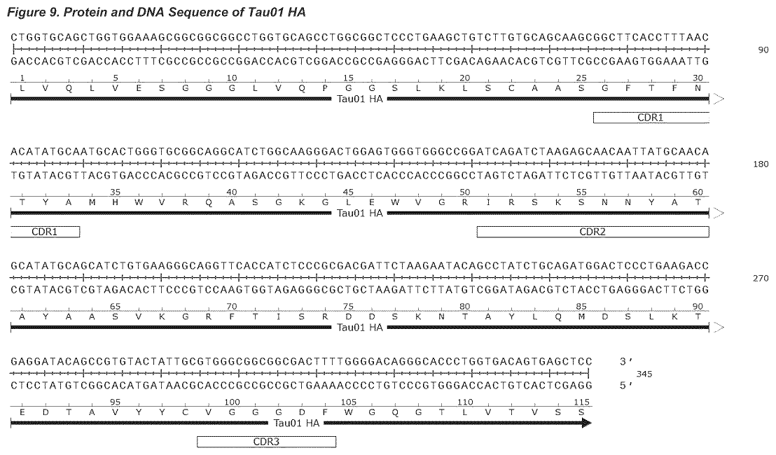

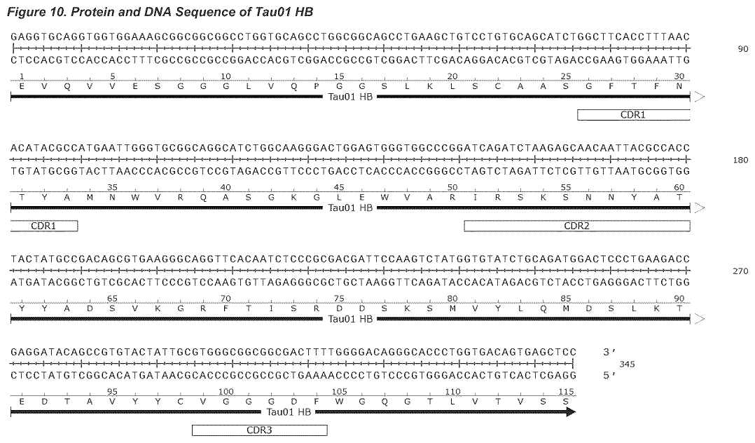

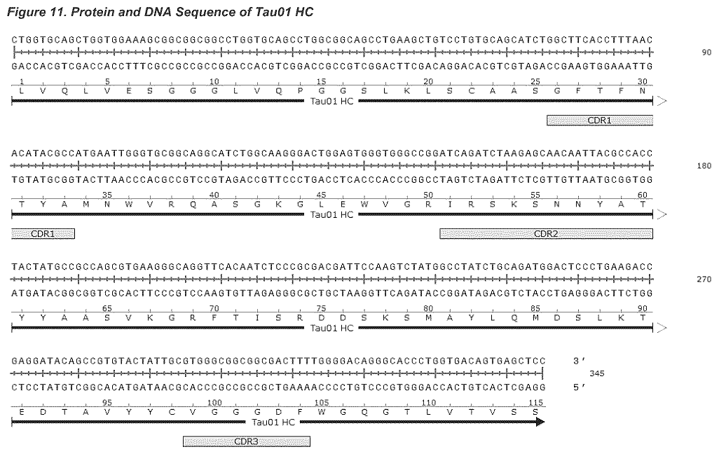

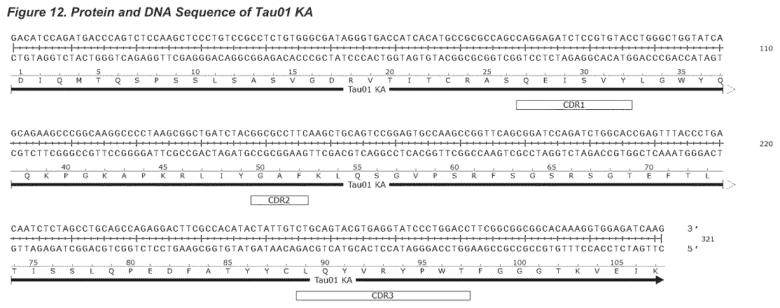

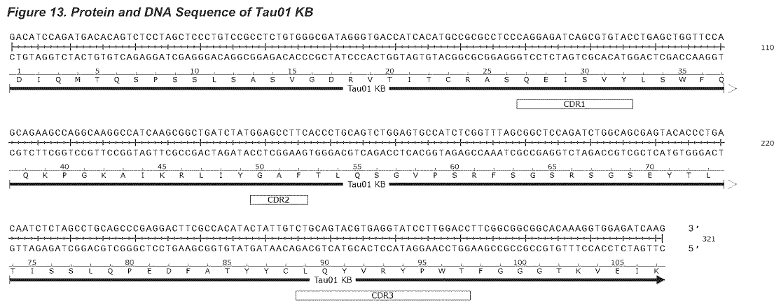

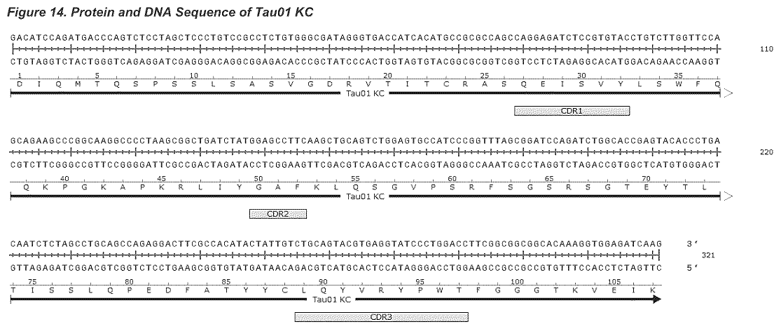

Clones were sequenced and plasmid DNA was prepared using the QIAGEN Plasmid Miniprep Kit or Qiagen Plasmid Maxiprep kit. The expression construct sequences (HA, HB, HC, KA, KB and KC) are shown in FIGS. 9 to 14.

### Antibody Expression

Expression plasmid preparations encoding (humanized or chimeric) VH and VK were used to transfect ExpiCHO cells, cultured for 7 days in serum free media, whereupon the conditioned medium containing secreted antibody was harvested. The concentrations of IgG4κ antibodies in ExpiCHO cell conditioned media were measured by octet and are shown in Tables 14 A-C. Most antibodies were produced at good expression levels.

TABLE 14
IgG levels in transfected ExpiCHO cell conditioned medium

| | IgG |
|---|---|
| | Concentration |
| | (μg/ml) |
| A | |
| MoTau01 | 181.4 |
| HuG4K | |
| Tau01 | 357.1 |
| HAKA | |
| Tau01 | 353.7 |
| HAKB | |
| Tau01 | 68* |
| HAKC | |
| Tau01 | 397.9 |
| HBKA | |
| Tau01 | 562.2 |
| HBKB | |
| Tau01 | 67* |
| HBKC | |
| Tau01 | 695.4 |
| HCKA | |
| Tau01 | 490.4 |
| HCKB | |
| Tau01 | 624.4 |
| HCKC | |
| B | |
| Tau01 | 75.5 |
| HDKA | |
| Tau01 | 67.4 |
| HDKB | |
| Tau01 | 49 |
| HDKC | |
| Tau01 | 8.24* |
| HEKA | |
| Tau01 | 65.3 |
| HEKB | |
| Tau01 | 51.9 |
| HEKC | |
| Tau01 | 54.75 |
| HFKA | |
| Tau01 | 115.15 |
| HFKB | |
| Tau01 | 118.15 |
| HFKC | |
| Tau01 | 66.4 |
| HGKA | |
| Tau01 | 56.3 |
| HGKB | |
| Tau01 | 7.14* |
| HGKC | |
| Tau01 | 57.1 |
| HHKA | |
| Tau01 | 53.6 |
| HHKB | |
| Tau01 | 61.5 |
| HHKC | |
| Tau01 | 65.8 |
| HIKA | |
| Tau01 | 47.4 |
| HIKB | |
| Tau01 | 58.5 |
| HIKC | |
| Tau01 | 52.4 |
| HJKA | |
| Tau01 | 49.8 |
| HJKB | |
| Tau01 | 52.1 |
| HJKC | |
| Tau01 | 48 |
| HKKA | |
| Tau01 | 39.5 |
| HKKB | |
| Tau01 | 48.8 |
| HKKC | |
| Tau01 | 42.3 |
| HLKA | |
| Tau01 | 42.2 |
| HLKB | |
| Tau01 | 53.7 |
| HLKC | |
| C | |
| Tau01 | 20.9* |
| HBKD | |
| Tau01 | 61.9 |
| HBKE | |
| Tau01 | 74 |
| HBKF | |
| Tau01 | 84.1 |
| HBKG | |
| Tau01 | 60.4 |
| HBKH | |
| Tau01 | 49.6 |
| HBKI | |
| Tau01 | 83.3 |
| HBKJ | |
| Tau01 | 16.3* |
| HCKD | |
| Tau01 | 60.1 |
| HCKE | |
| Tau01 | 50 |
| HCKF | |
| Tau01 | 61 |
| HCKG | |
| Tau01 | 55.8 |
| HCKH | |
| Tau01 | 19.2* |
| HCKI | |
| Tau01 | 48.6 |
| HCKJ | |

*Expression of transfection control, Hu1210 HuG1K was also reduced (expected level ~100 μg/ml).

### Antigen Binding by Initial Versions of the Humanized Antibodies

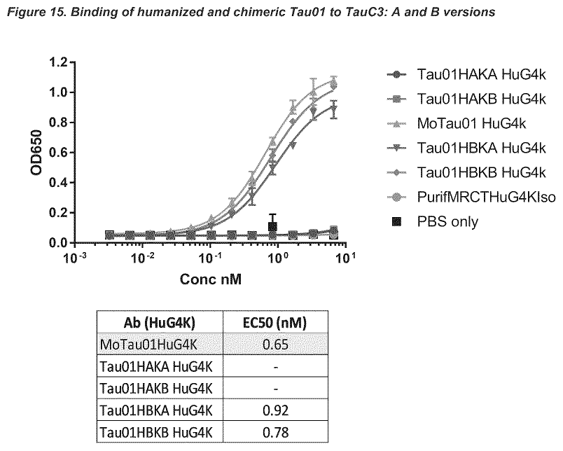

The binding of the humanized variants to the TauC3 antigen provided was tested by binding ELISA as described in Example 5. The data shown in FIG. 15 displays the binding ELISA of TauC3 with humanized antibodies consisting of the HA/HB heavy chains in combination with KA/KB light chains. The HAKA and HAKB humanized antibodies do not bind to TauC3. The MoTau01HuG4K chimeric binds to TauC3 with an EC50 value of 0.65 nM and the HBKA and HBKB variants bind with similar EC50 values with the HBKB version being the closest (0.78 nM).

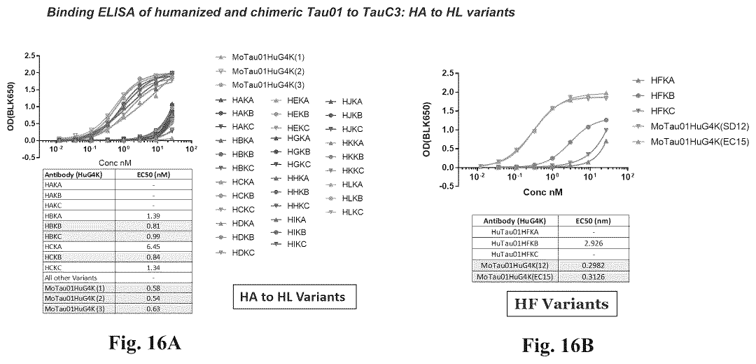

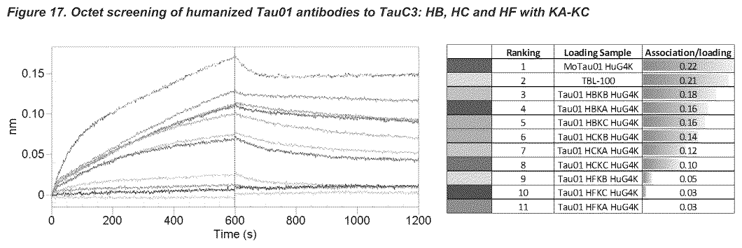

Considering this data, further versions of the humanized heavy and light chains were expressed, each with a single back-mutation (Tables 8 and 12). FIG. 16 shows the results of the heavy chain single mutants and the HC version in combination with KA-KC, tested for binding to TauC3 by ELISA. The data shows the heavy chain single mutants were unable to bind as well as the chimeric antibody to TauC3. The HB and HC versions in combination with KA-KC bound to TauC3 with the highest EC50 values with HBKB and HBKC displaying the closest value to the chimeric antibody (0.81 nM and 0.84 nM respectively). These results were confirmed using a screening assay on the Octet instrument with one concentration of TauC3 as described in section 8.12 (FIG. 17). The Octet data is not optimal as two-phase association and dissociation events can be observed which is likely due to the nature of the TauC3 protein. However, the octet data is sufficient for screening and ranking the humanized candidates.

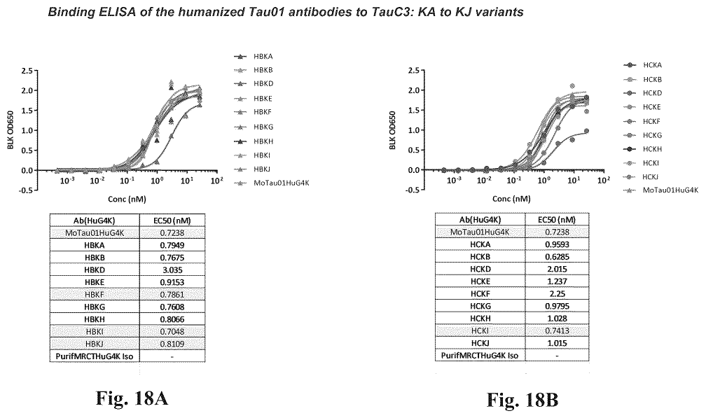

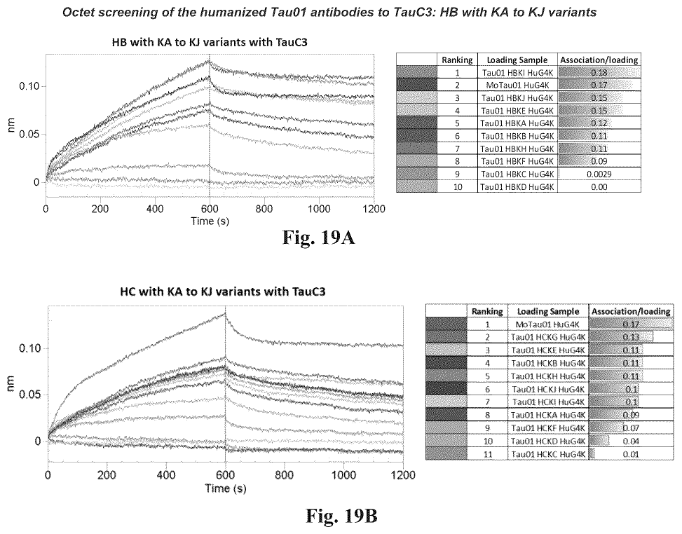

Since HB/HC are the best heavy chains versions, their binding to TauC3 in combination with all the light chain versions (KA-KJ) was tested by binding ELISA (FIG. 18) and the Octet screening assay (FIGS. 19 A and B). Many of the light chain single mutants are capable of retaining binding to TauC3 but the light chain versions KE, KG, KI and KJ rank the highest in both assays.

### Design and Generation of the Second Round of the Humanized Antibodies

Based on the sub-optimal binding results for the initial humanized variants, a second round of variants were designed by incorporating additional back-mutations into the HC and KC versions (HM-HO and KL and KM respectively) or combining two back-mutations in the KA version (KN-KP) (Tables 8 and 12). The mutagenesis, DNA preparation, expression and quantitation were conducted. The expression levels obtained are shown in Tables 14 D and E. Most antibodies were produced at good expression levels.

TABLE 14
IgG levels in transfected ExpiCHO cell conditioned medium

| | IgG |
|---|---|
| | Concentration |
| | (μg/ml) |
| D | |
| Tau01 | 377.95 |
| HMKA | |
| Tau01 | 329.85 |
| HMKB | |
| Tau01 | 552.3 |
| HMKC | |
| Tau01 | 399.5 |
| HMKG | |
| Tau01 | 417.9 |
| HMKJ | |
| Tau01 | 419.2 |
| HMKI | |
| Tau01 | 441.4 |
| HNKA | |
| Tau01 | 557.8 |
| HNKB | |
| Tau01 | 50.45 |
| HNKC | |
| Tau01 | 487 |
| HNKG | |
| Tau01 | 662.8 |
| HNKJ | |
| Tau01 | 370.5 |
| HNKI | |
| Tau01 | 354.85 |
| HOKA | |
| Tau01 | 352.75 |
| HOKB | |
| Tau01 | 92.85 |
| HOKC | |
| Tau01 | 321.8 |
| HOKG | |
| Tau01 | 333.6 |
| HOKJ | |
| Tau01 | 262.15 |
| HOKI | |
| E | |
| Tau01 | 353.7 |
| HCKL | |
| Tau01 | 261.75 |
| HCKM | |
| Tau01 | 406.7 |
| HCKN | |
| Tau01 | 710.4 |
| HCKO | |
| Tau01 | 677.5 |
| HCKP | |
| Tau01 | 535.6 |
| HMKE | |
| Tau01 | 449.8 |
| HMKL | |
| Tau01 | 164.43 |
| HMKM | |
| Tau01 | 443.7 |
| HMKN | |
| Tau01 | 431.7 |
| HMKO | |
| Tau01 | 352.1 |
| HMKP | |

### Antigen Binding by the Second Round of the Humanized Tau01 Antibodies

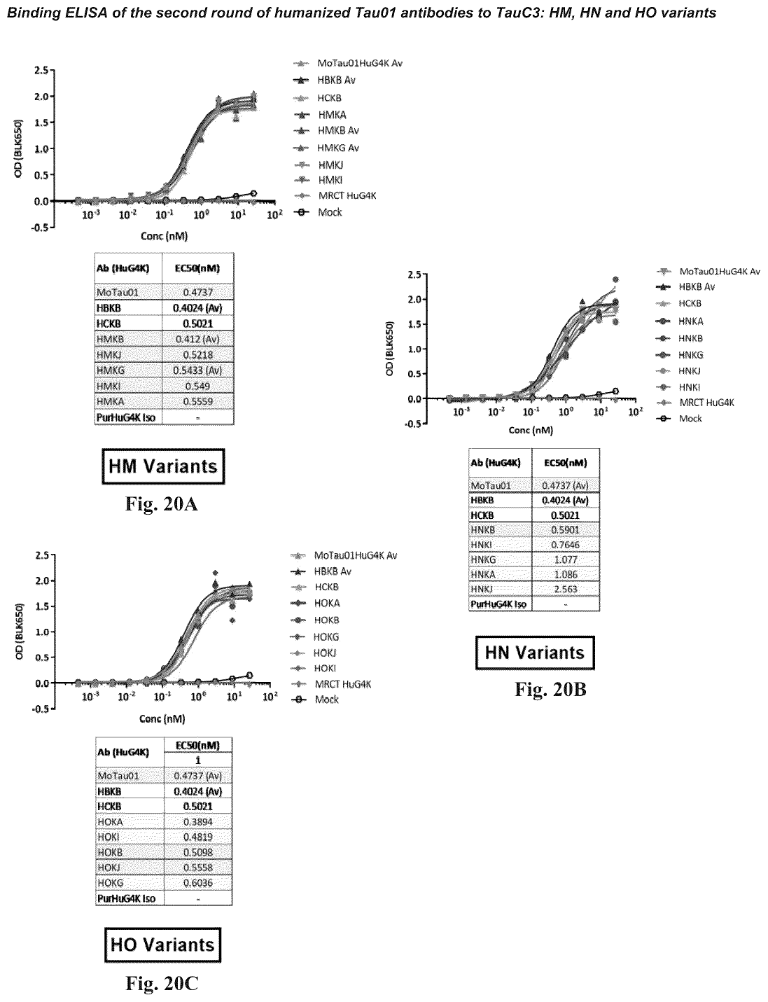

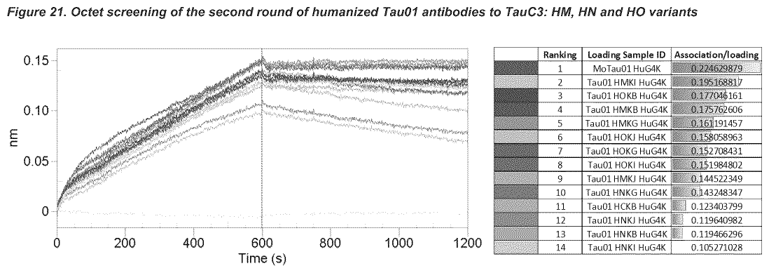

In order to evaluate the heavy chain, the binding activity of antibody variants containing HM, HN and HO combined with light chains KA, KB, KG, KI, KJ were tested by ELISA (FIG. 20) and Octet (FIG. 21) against TauC3. The HM and HO variants ranked the highest in both assays. The only difference between the HO and HM variant is an additional back-mutation of L-V in position 4 which is also present in the HN variant. Since the HN versions did not bind as well as HM versions, the L-V back-mutation was not deemed to be necessary so HM was chosen as the top heavy chain from the second round of designs.

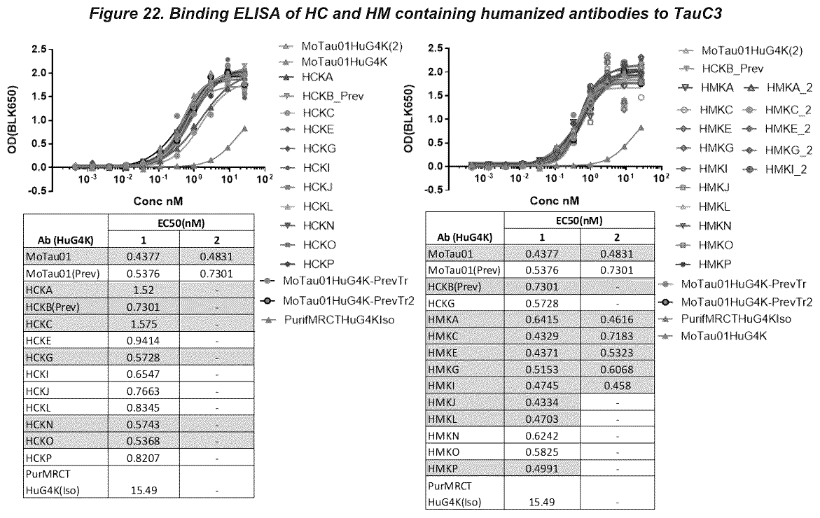

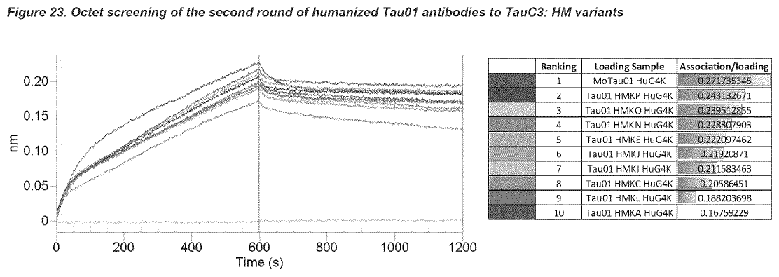

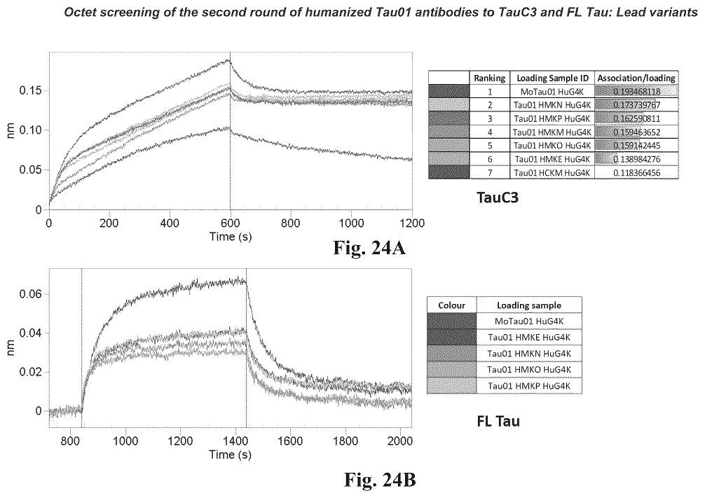

The lead heavy chain HM was expressed with several light chains; KA-KC, the selected single-mutants previously highlighted (KE, KG, KI, KJ) and the second round of designs, KL, KN, KO, KP. These lights chains were also expressed in combination with the earlier favored heavy chain, HC, for comparison. The binding of these humanized antibody variants was tested by binding ELISA (FIG. 22) against TauC3. Overall, the HM variants bound with higher EC50s than the HC variants so the former variants were evaluated in the Octet screening assay (FIG. 23). The antibody variants HMKE, HMKN, HMKO and HMKP ranked the highest in both assays. The KM light chain was also screened in combination with HC/HM and compared to the leading antibody variants by Octet (FIG. 24 A). The HMKM version ranked very highly in the Octet screening assay whereas HCKM ranked the lowest. The binding of the humanized leads was also tested against FL Tau using Octet to determine how much selectivity had been retained (FIG. 24 B). The signals obtained with 500 nM FL Tau were very low (0.03-0.07 nm) which is in the non-specific binding range.

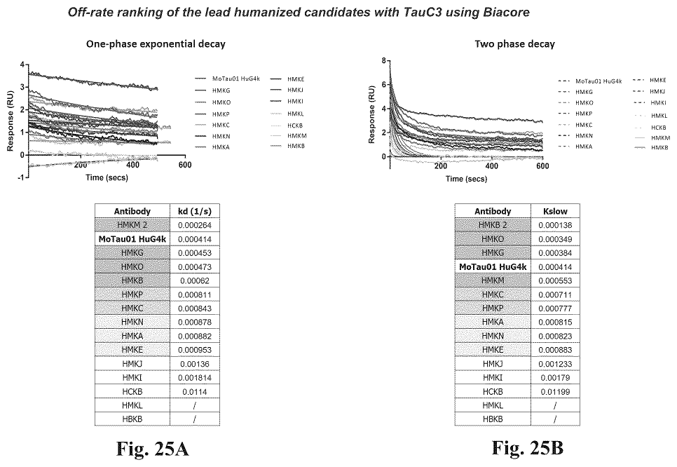

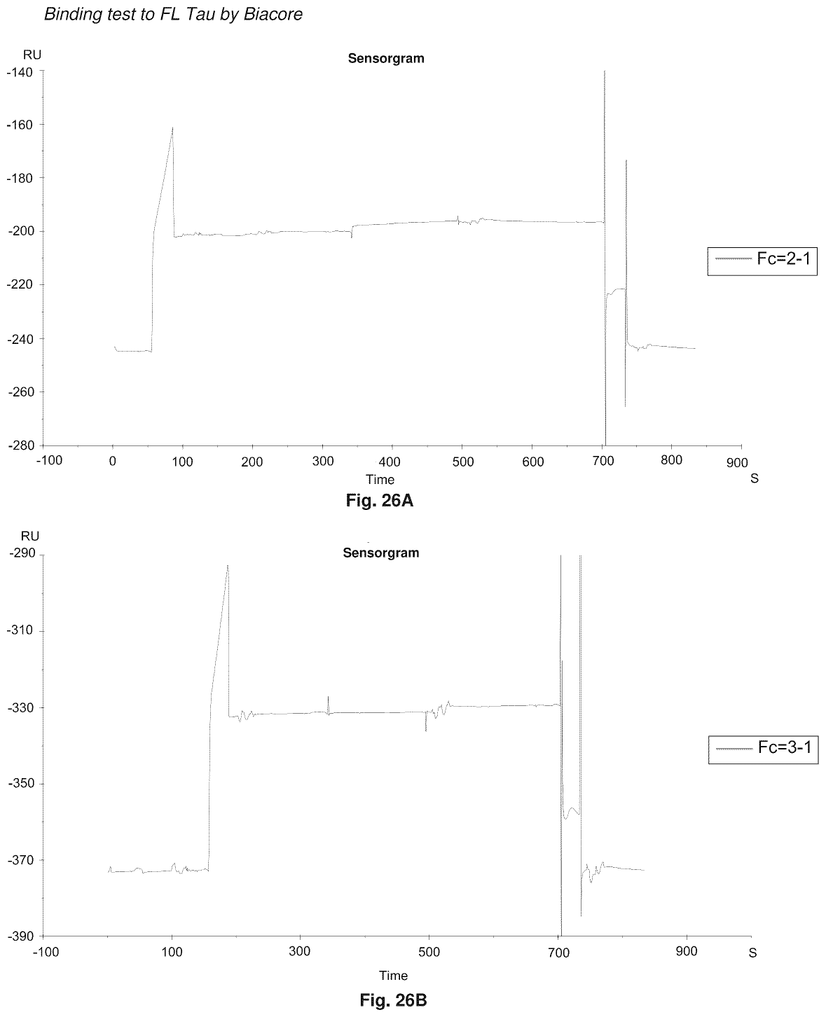

To further evaluate the binding data observed on the Octet, an off-rate ranking experiment was performed on the Biacore 200 instrument. Biacore offers greater selectivity than the Octet and facilitated the immobilization of TauC3 onto a CM5 chip via amine coupling which stabilizes the antigen. This produces higher quality data with stable baselines and good reproducible responses. Once TauC3 is immobilized, one concentration (5 nM) of antibody is added, followed by a dissociation and regeneration step (Section 8.20). The off-rates were fitted using a two-phase decay model (FIG. 25 B) or a one phase decay fitting only the second off-rate (FIG. 25 A). The candidates were then ranked based on the off-rates obtained using the fits and compared. The data agreed with the Octet results (albeit with a different ranking order), highlighting Tau01 HMKM, HMKO, HMKP, HMKN and HMKE as the humanized variants with the slowest off-rates. The lack of binding of FL Tau to chimeric and a humanized candidate was also confirmed by Biacore by loading the antibody onto a Protein G chip and adding 250 nM FL Tau. FIG. 26 shows no binding could be observed for either MoTau01 HuG4k or Tau01 HCKB.

### Thermal Stability of the Humanized Tau01 Candidate Antibodies

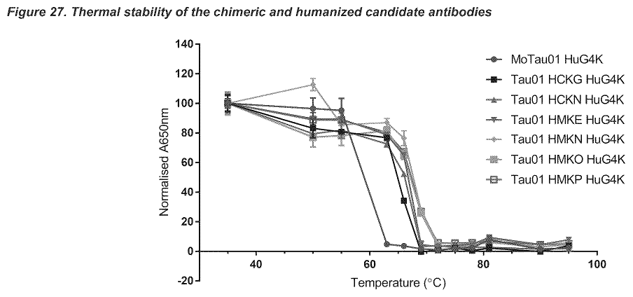

The aim of this experiment was to test the thermal stability of the chimeric antibody and some of the humanized antibodies (Tau01 HCKG, HCKN, HMKE, HMKN, HMKO, HMKP) when subjected to higher temperatures, varying from 35° C. to 95° C. for 10 minutes, cooled to 4° C. and used in a binding ELISA at the EC80 concentration of each candidate (Section 8.15). All humanized candidate antibodies were more stable than the chimeric antibody, retaining binding ability to TauC3 until 67-68° C. following which binding to TauC3 decreased (FIG. 27). The humanized variants exhibit increased thermal stability compared to the chimeric antibody MoTau01 HuG4k which only retained binding up to −55° C. The variants containing the HM heavy chain retained binding to slightly higher temperatures than the HC heavy chain variants tested.

### Selection of Lead Humanized Tau01 Candidate Antibodies

Taking all of these results together, the lead humanized antibody variants Tau01 HMKE, HMKM, HMKN, HMKO and HMKP were selected to scale-up and purify using affinity and size-exclusion chromatography, as described in Section 8.16. The purified antibodies were further characterized in a series of biophysical assays. The HCKB humanized candidate with weaker binding to TauC3 was also scaled up for expression and purification in order to be used as a comparator antibody for ranking in the seeding assay. This would allow us to further test the correlation between antibody affinity in vitro and potency in the cell assay.

### Aggregation of Humanized Tau01 Candidate Antibodies

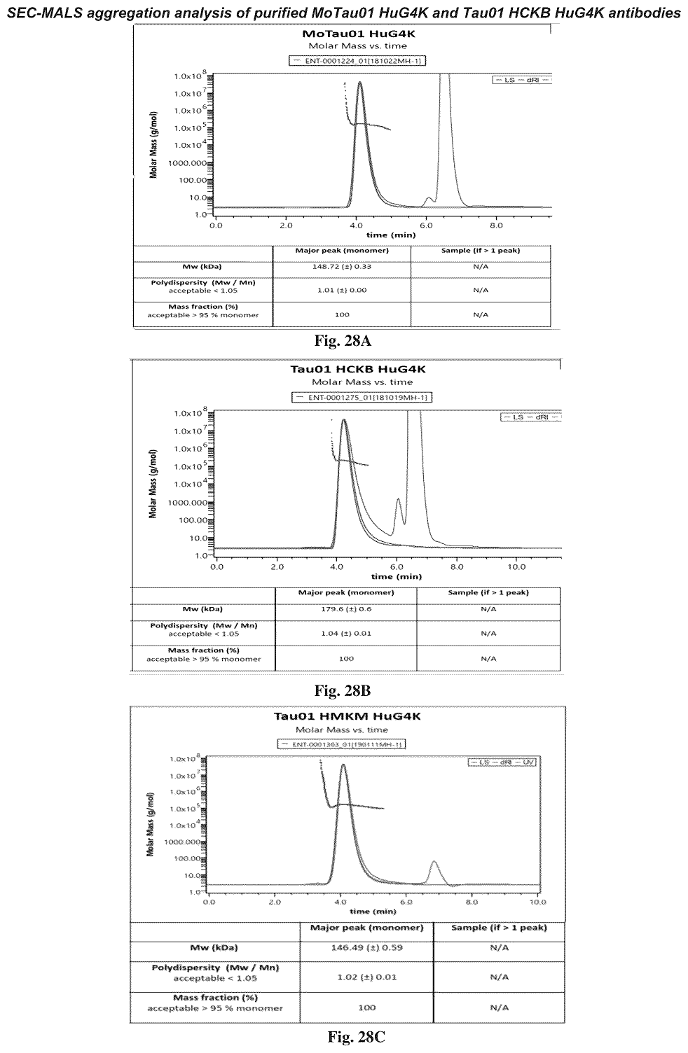

As part of the QC process after purification, the antibody samples are subjected to SEC-MALS/DLS followed by mass spectrometry. To determine the absolute molar masses and check for aggregation, the purified antibody samples were injected into a size exclusion column in an HPLC system and analyzed by multi-angle light scattering. The profiles for MoTau01 HuG4k and Tau01 HMKM show no signs of aggregation with an average molecular weight of about 147-8 kDa, which is the expected range for an IgG monomer in this analysis setup (FIG. 28 A). The Tau01 HCKB antibody profile has a broad peak which skews the data resulting in a molecular weight of 179.6 kDa (FIG. 28 B). All three antibodies are monodisperse (Mw/Mn<1.05) and display no signs of aggregation.

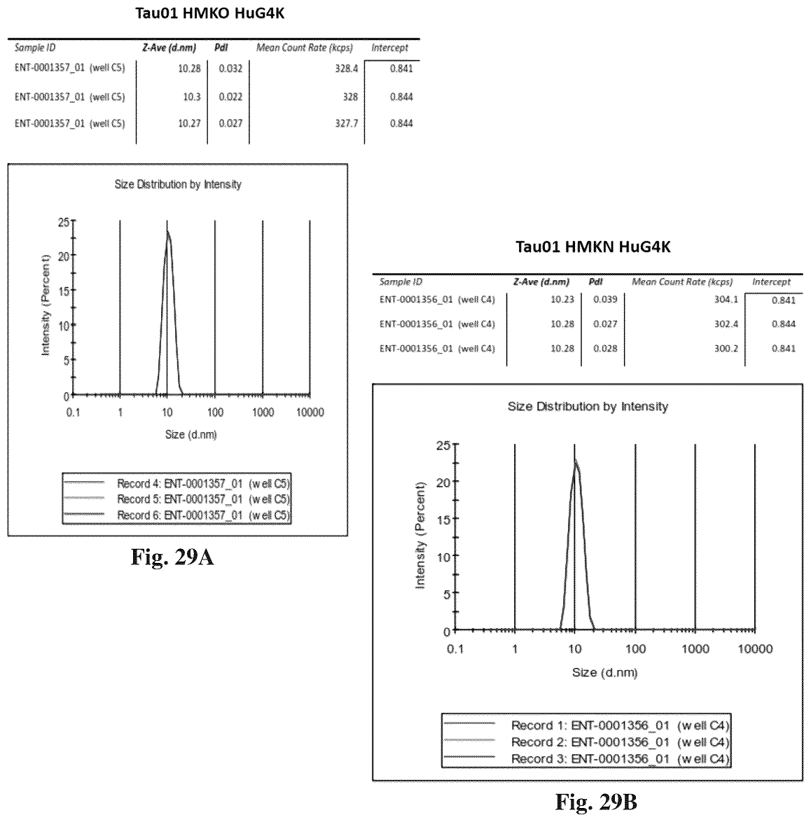

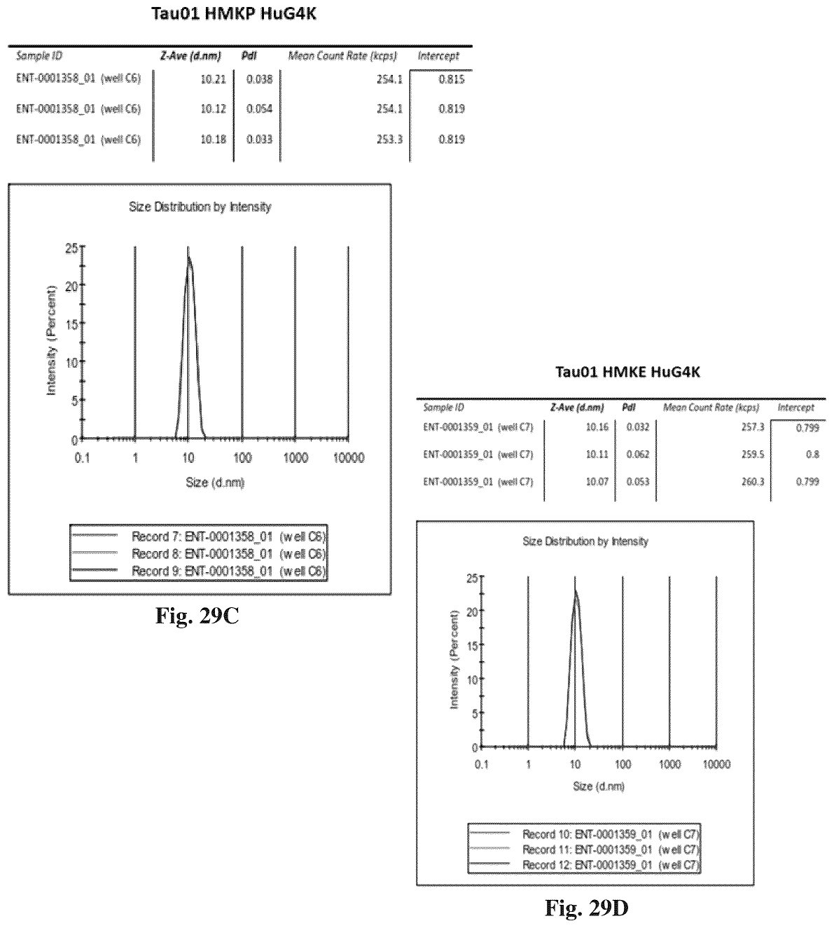

Dynamic light scattering is a complementary technique to static-light scattering (SEC-MALS) for the detection of soluble aggregates and was used to QC the humanized variants Tau01 HMKN, HMKO, HMKP and HMKE. The Z-Ave or hydrodynamic diameter is expected to be around 10 for an antibody and the Polydispersity index (PdI) should be <0.1 if the sample is monodisperse. As shown in FIG. 29, all of the antibody samples contain one major population which is monodisperse and are consistent with the size of a monoclonal antibody.

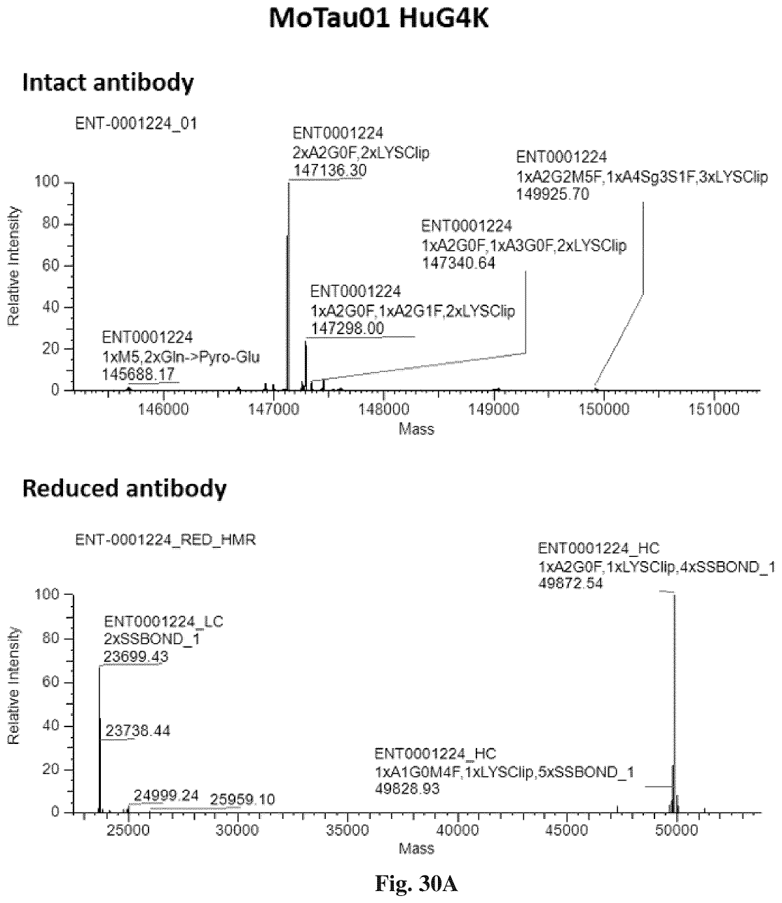

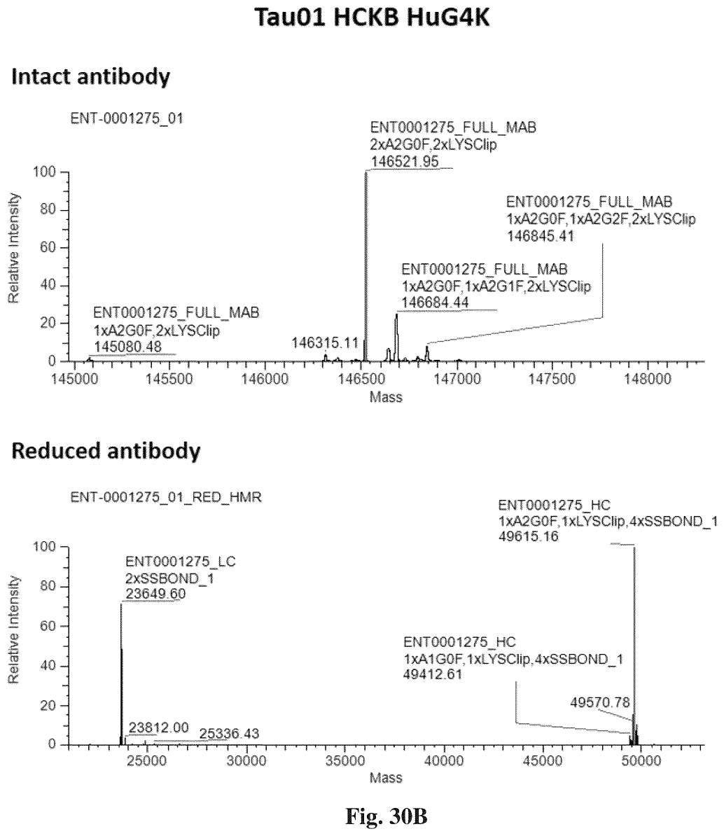

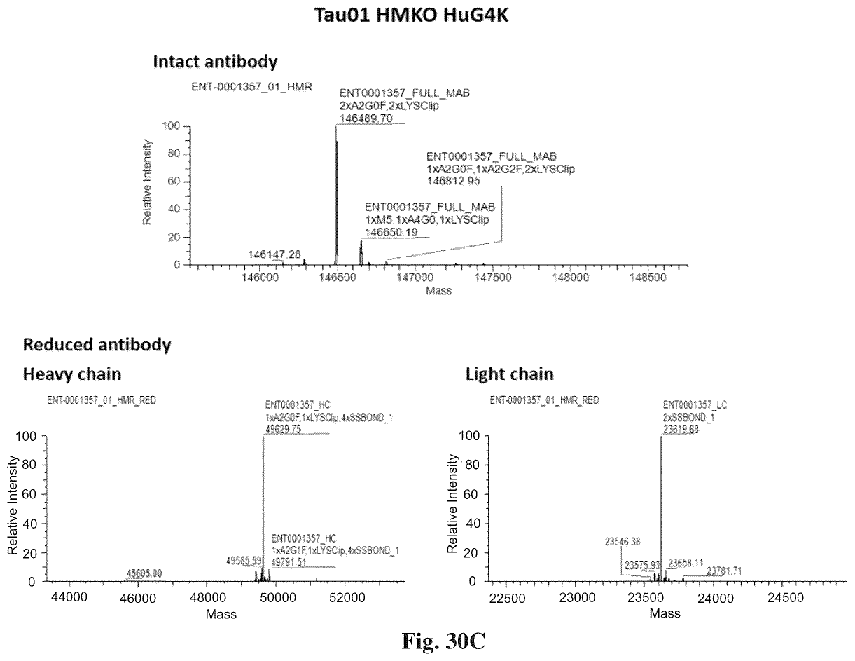

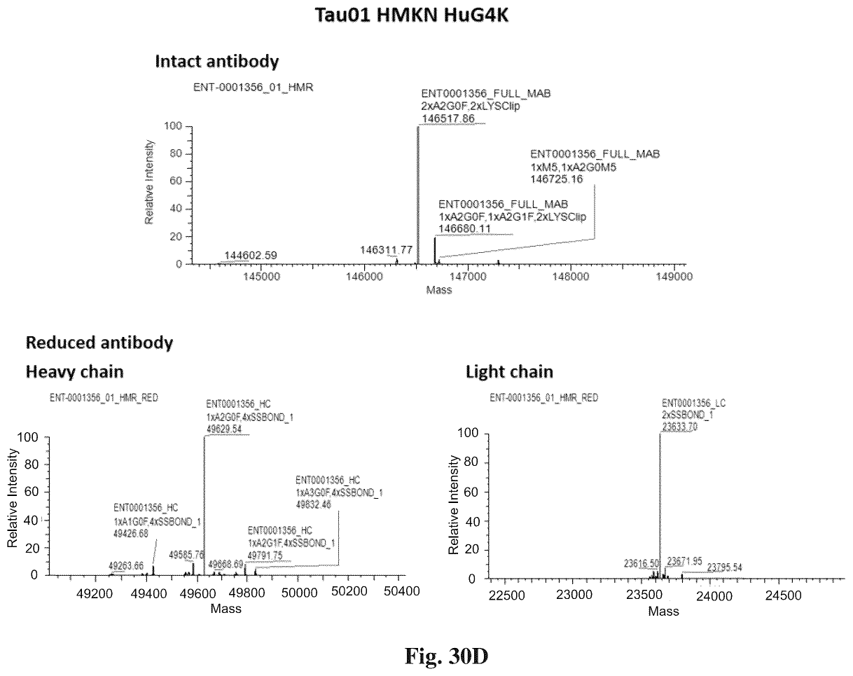

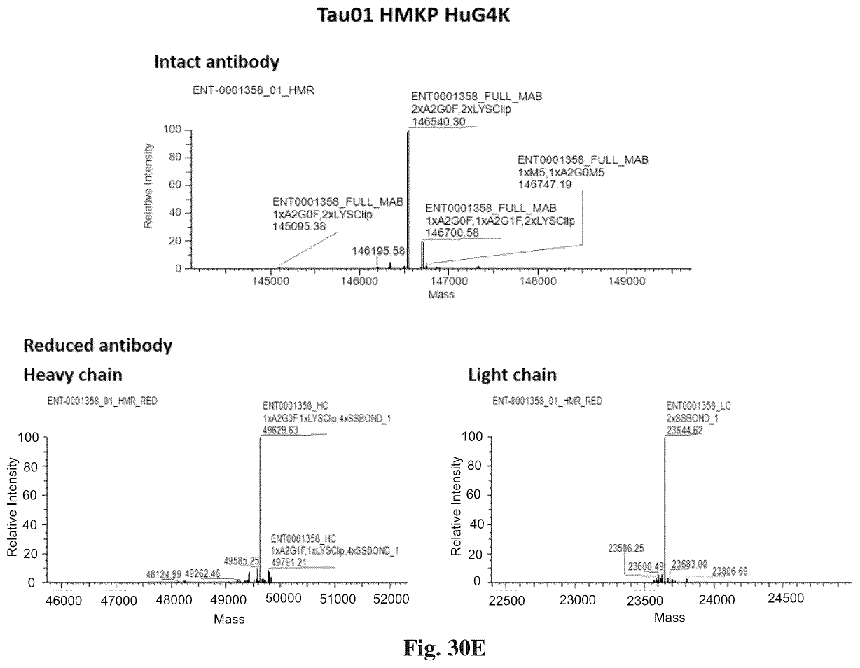

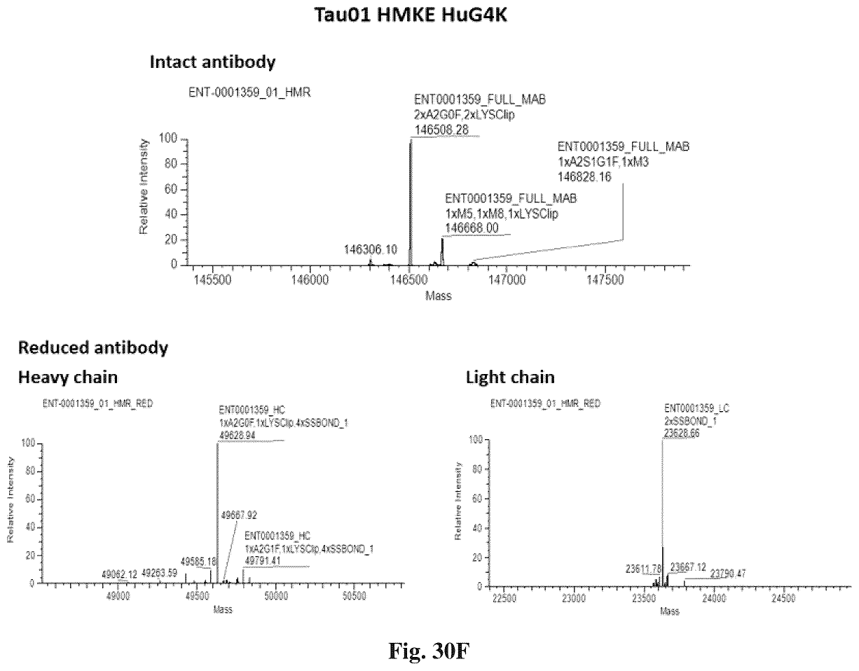

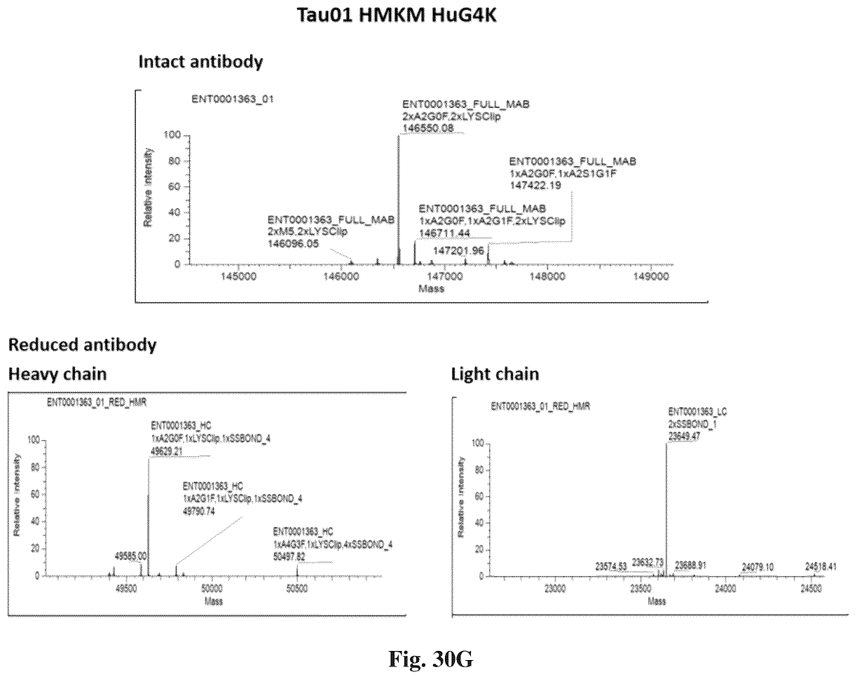

To confirm the accurate molecular weight of the antibodies, mass spectrometry was performed on intact and reduced antibody samples and are shown in FIG. 30 A-G. The molecular weights match the predicted molecular weights for all of the antibodies tested and the amino acid sequences confirmed. No other liabilities were flagged. Overall, the purified chimeric and humanized antibodies passed QC.

### Kinetic Studies of the Humanized Tau01 Candidate Antibodies to TauC3

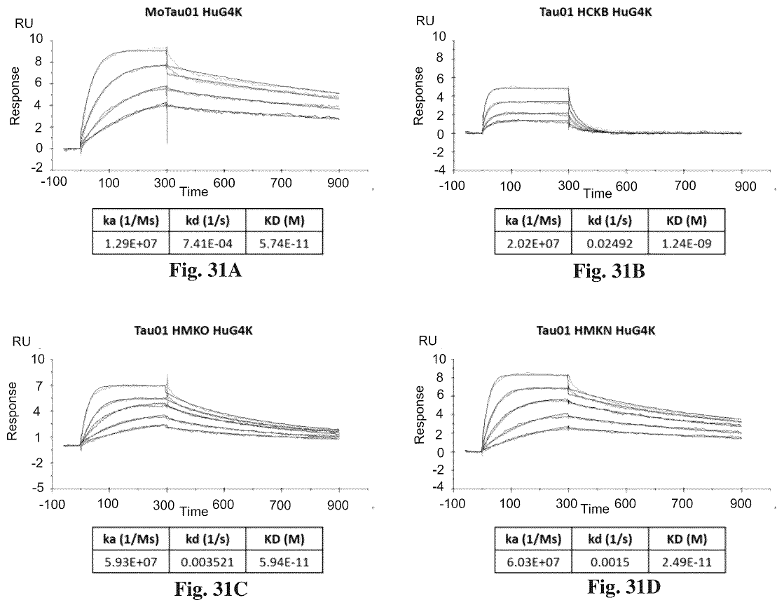

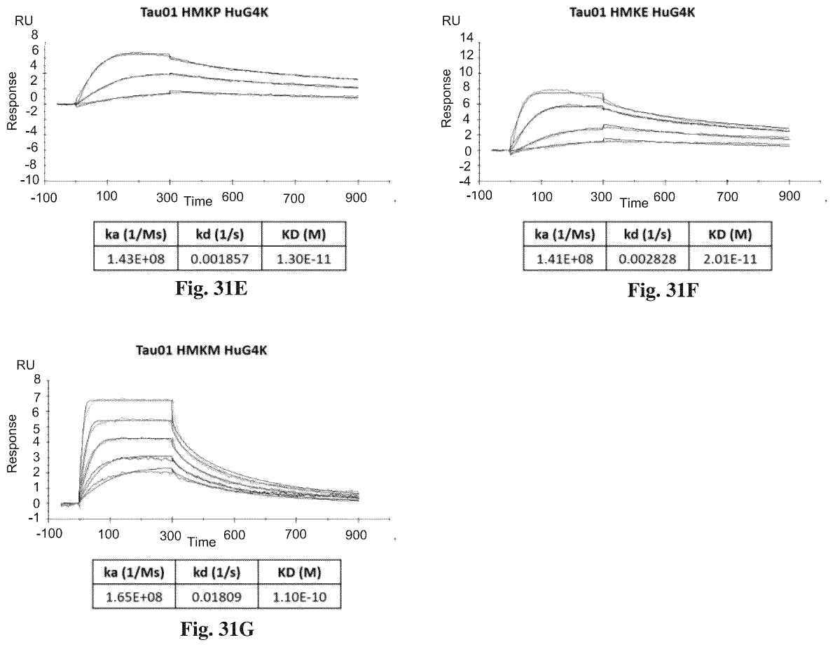

To determine the affinity of the binding interactions, a biacore kinetic assay was developed which involved immobilizing TauC3 on a CM5 chip via amine coupling and injecting a concentration series of the respective antibody over it. The chimeric antibody binds to TauC3 with a KD of 57 pM (FIG. 31 A).

FIG. 31 B shows Tau01 HCKB is the weakest binder to TauC3 (1.2 nM) followed by HMKM which binds to TauC3 with an affinity of 110 pM (FIG. 31 G). Tau01 HMKO, HMKN, HMKP and HMKE, shown in FIG. 31 C-F, have KD values comparable or tighter than the chimeric antibody. However, the off-rate is slightly faster for all humanized candidates compared to the chimeric (0.001-0.004 for humanized vs 0.0007 for the chimeric). It should be noted that the ka is at the limits of the instrument so the absolute values should be compared with caution. It can be concluded that the antibodies HMKN, HMKP and HMKE bind in the picomolar range and have the slowest off-rates of all the humanized variants tested.

### Determination of the Melting Temperature (Tm) of Humanized Tau01 Candidate Antibodies

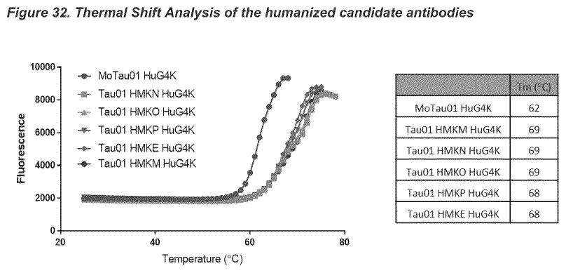

In order to determine the melting temperature of the lead antibodies Tau01 HMKE, HMKM, HMKN, HMKO, HMKP a thermal shift assay was performed. Samples were incubated with a fluorescent dye (Sypro Orange) for 71 cycles with 1° C. increase per cycle in a qPCR thermal cycler (Section 8.21). Tm for the humanized antibodies were calculated to be 68-69° C. (FIG. 32).

### Non-Specific Protein-Protein Interactions (CIC) of Humanized Tau01 Candidate Antibodies

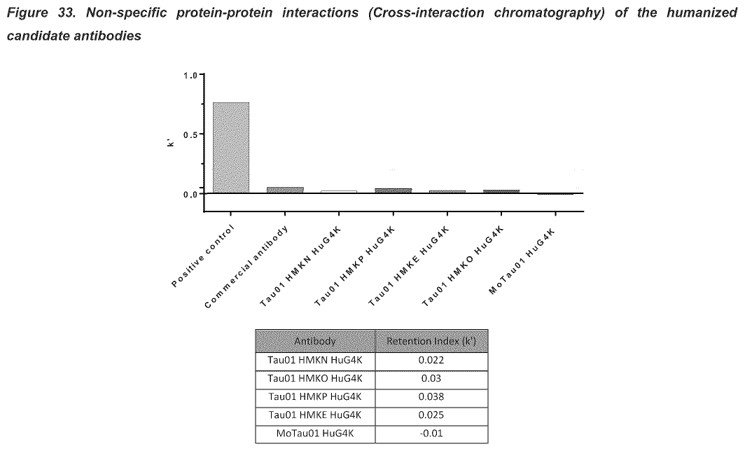

Cross-Interaction Chromatography using bulk purified human polyclonal IgG is a technique for monitoring non-specific protein-protein interactions and can provide an indication of any solubility issues, which can give rise to downstream manufacturing problems, as explained in Example 5. An elevated Retention Index (k′) indicates a self-interaction propensity and a low solubility. Humanized Tau01 HMKE, HMKM, HMKN, HMKO, HMKP candidate antibodies show a Retention Index below 0.038, indicating a low propensity for non-specific interactions and good solubility (FIG. 33).

### Solubility of Humanized Tau01 Candidate Antibodies

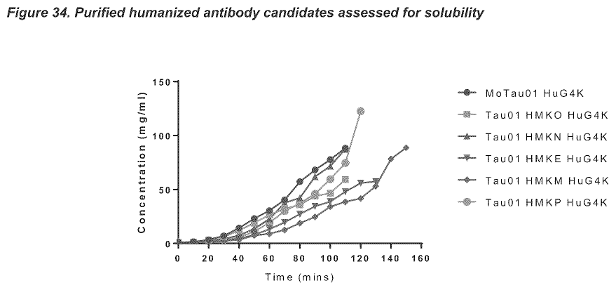

The humanized Tau01 HMKE, HMKM, HMKN, HMKO, HMKP candidate antibodies were concentrated using solvent absorption concentrators (MWCO 7500 kDa) and the concentration measured at timed intervals. Tau01 HMKP was concentrated to 123 mg/ml and Tau01 HMKN, HMKM and MoTau01 HuG4K antibodies were concentrated to 87-88 mg/mL (FIG. 34). Tau01 HMKO HuG4K was concentrated to 59 mg/ml without apparent precipitation and Tau01 HMKE HuG4K was concentrated to 57 mg/ml. The data suggest that the antibodies are not prone to precipitation at concentrations of up to 57 mg/mL.

### Freeze/Thaw and Heat Stress Analysis of Humanized Tau01 Candidate Antibodies by Circular Dichroism

Circular Dichroism (CD) is a spectroscopic technique which allows us to observe the overall secondary structure of a purified protein sample.

The freeze-thaw (FT) stress experiment involved subjecting samples of the purified candidate antibodies to 10 cycles of 15 minutes at −80° C. followed by thawing for 15 minutes at room temperature. To perform the heat stress experiment, samples of the purified candidate antibodies were exposed to temperatures of; a) 4° C., b) room temperature (RT), c) 37° C. and d) 50° C. for 25 days.

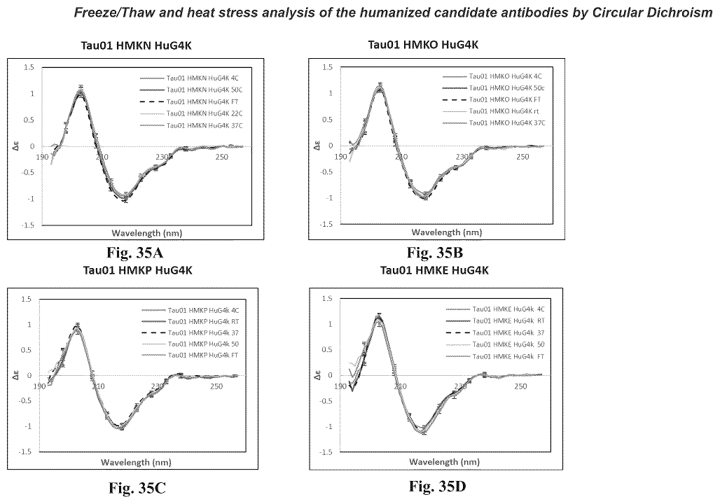

Samples were then analyzed by Circular dichroism to check if secondary structure had been retained (FIG. 35). All the humanized variants tested passes our internal threshold.

Overall, the data suggests that the heat stress and freeze/thaw cycles do not affect the secondary structure of the humanized Tau01 HMKE, HMKM, HMKN, HMKO, HMKP candidate antibodies.

### Isoelectric Point Analysis of Humanized Tau01 Candidate Antibodies

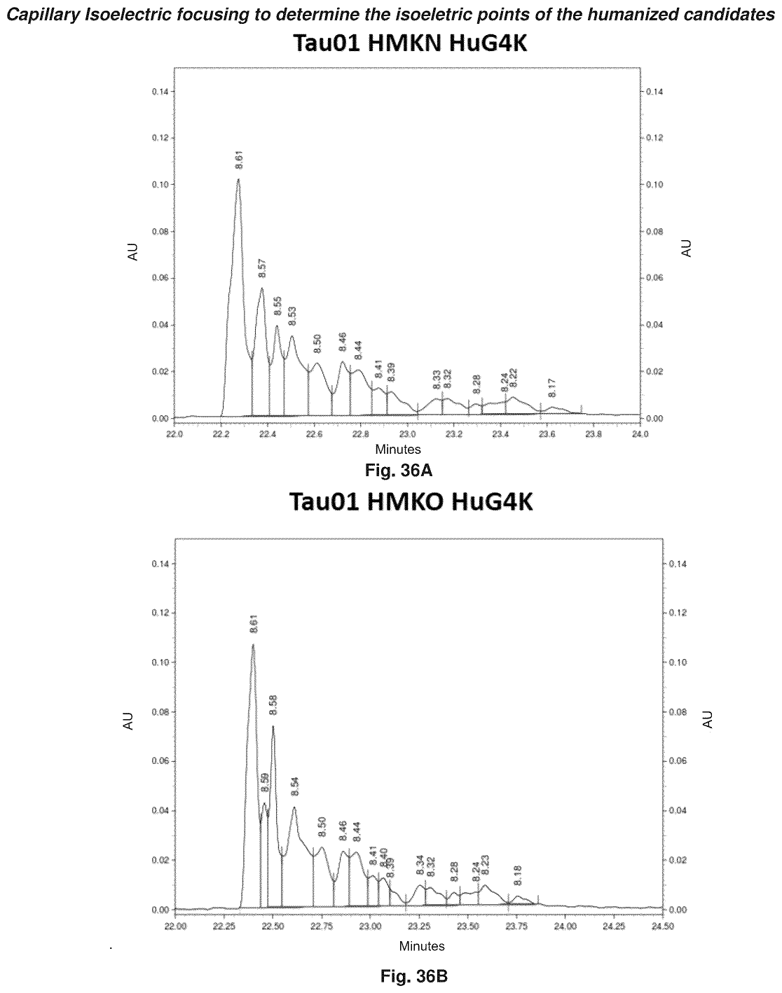

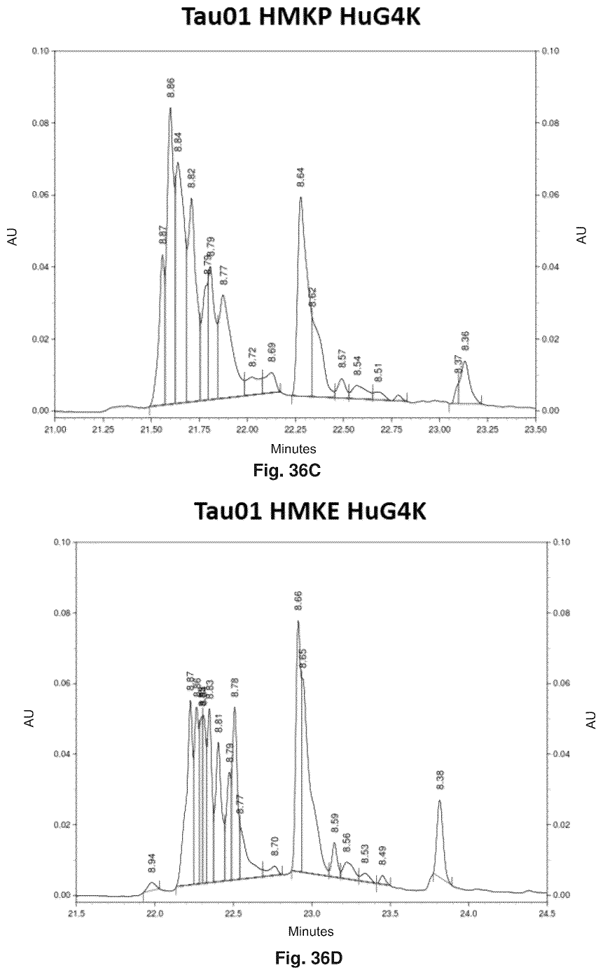

pI analysis of the humanized candidate antibodies was performed using capillary isoelectric focusing (cIEF). This technique allows antibodies to be separated according to their isoelectric point (pI) using a pH gradient across the capillary. FIG. 36 shows the chromatograms and Table 15 displays the main pI isoform(s) of each antibody (defined as greater than 10% peak area) and the pI range for each antibody. The main isoelectric points for humanized Tau01 HMKE, HMKM, HMKN, HMKO, HMKP candidates are ˜8.86-8.81.

### Serum Stability Assessment of Humanized Tau01 Candidate Antibodies

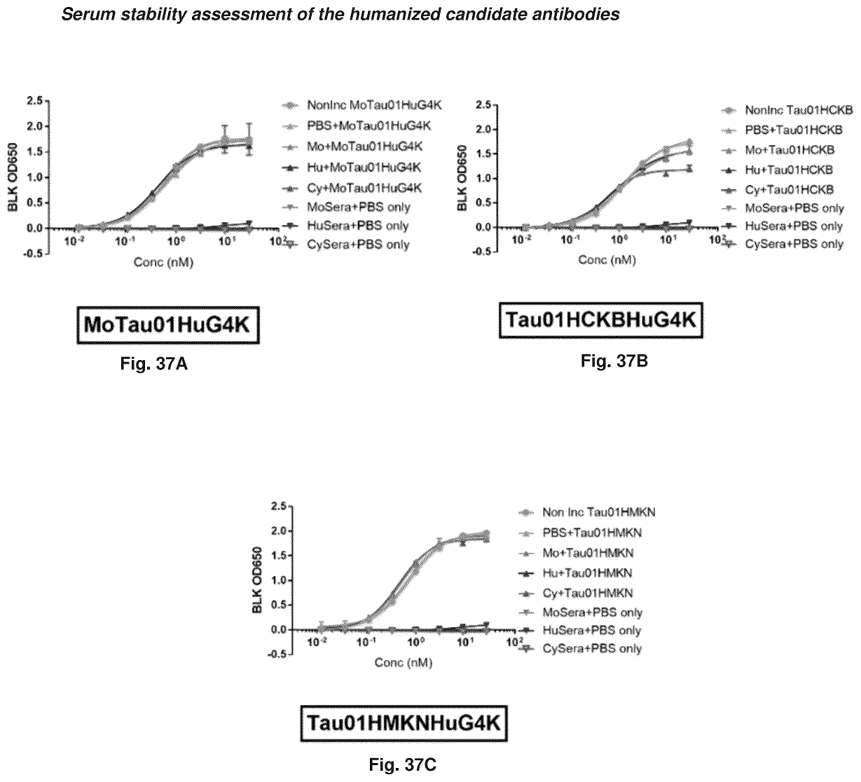

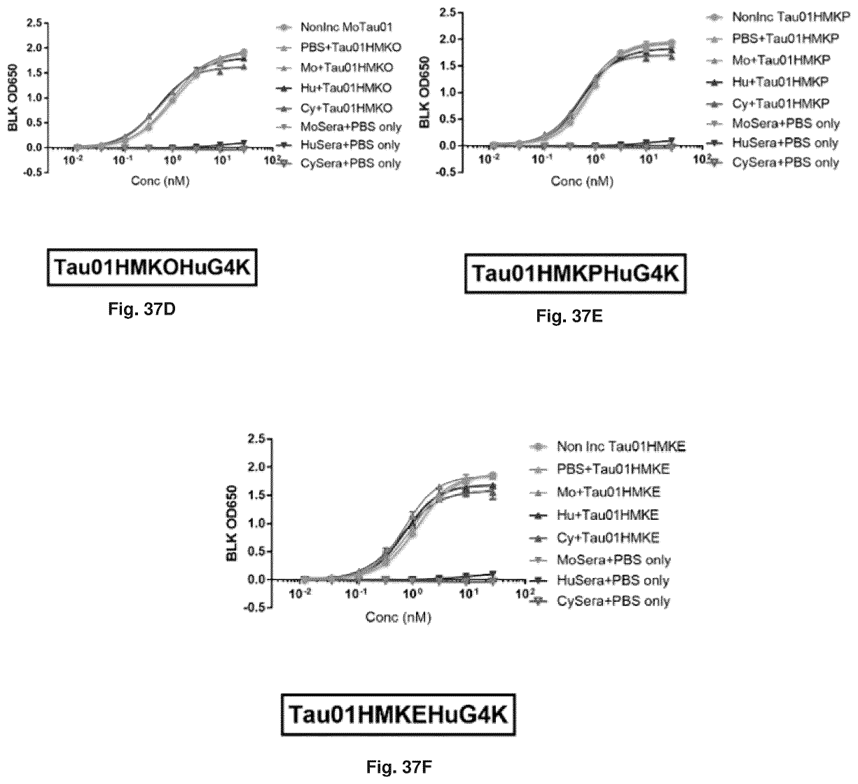

Purified samples of the chimeric and humanized antibodies were incubated in mouse, human and cynomolgus serum for 21 days. The binding of the Tau01 HMKE, HMKM, HMKN, HMKO, HMKP candidate antibodies which had been incubated in the 3 different serums were compared with an antibody incubated in PBS and a 4° C. positive control sample by binding ELISA to TauC3 (FIG. 37). The Tau01 HMKE, HMKM, HMKN, HMKO, HMKP candidate antibodies retained their binding capability after being incubated in mouse, human and cynomolgus serum.

### Summary of Data for the Lead Tau01 HMKE HMKN, HMKO, HMKP, HMKM Humanized Candidates Compared to MoTau01 HuG4k

Table 16 shows a summary of the binding, kinetic affinity and biophysical properties of the lead humanized Tau01 HMKE HMKN, HMKO, HMKP, HMKM antibody candidates compared to the chimeric antibody MoTau01 HuG4k. All of the humanized candidates bind in the picomolar range but of these, Tau01 HMKN, HMKO and HMKP have the highest affinities and slowest off-rates and also pass all of our biophysical assays. Taking all the data into account, Tau01 HMKP was chosen as the lead humanized candidate as it has the highest affinity, a slow off-rate and shows no potential liabilities in the biophysical assays. Tau01 HMKN and Tau01 HMKO and KMKE are all good back up humanized lead candidates as they also have excellent properties.

TABLE 16
Final humanized candidate antibody summary

| | | | Tau01 | | Tau01 | | Tau01 | | Tau01 | | Tau01 | |
|---|---|---|---|---|---|---|---|---|---|---|---|---|
| | MoTau01 | | HMKN | | HMKO | | HMKP | | HMKE | | HMKM | |
| | HuG4k | | HuG4k | | HuG4k | | HuG4k | | HuG4k | | HuG4k | |
| Expression | 213.2 | mg/L | *40 | mg/L | *44 | mg/L | 153 | mg/L | 262.5 | mg/L | 221.2 | mg/L |
| ka (1/Ms) | 1.29 × 10⁷ | | 6.03 × 10⁷ | | 5.93 × 10⁷ | | 1.43 × 10⁷ | | 1.41 × 10⁸ | | 1.65 × 10⁸ | |
| kd (1/s) | 7.41 × 10⁻⁴ | | 1.50 × 10⁻³ | | 3.52 × 10⁻³ | | 1.86 × 10⁻³ | | 2.83 × 10⁻³ | | 1.81 × 10⁻² | |
| KD (pM) | 57 | | 25 | | 59 | | 13 | | 20 | | 110 | |
| pI range | 6.68-7.16 | | 8.17-8.61 | | 8.18-8.61 | | 8.36-8.87 | | 8.38-8.94 | | 8.29-8.57 | |
| Average Tm | 62 | | 69 | | 69 | | 68 | | 68 | | 69 | |
| (° C.) | | | | | | | | | | | | |
| Thermal | <60° C. | | >67° C. | | >67° C. | | >67° C. | | >67° C. | | / | |
| stability | | | | | | | | | | | | |
| Solubility | ≥80 | mg/ml | ≥80 | mg/ml | ≥50 | mg/ml | ≥100 | mg/ml | ≥50 | mg/ml | ≥80 | mg/ml |

| | | | | | | |
|---|---|---|---|---|---|---|
| CD-50° C. | No data | pass | pass | pass | pass | pass |
| incubation | | | | | | |
| CD-repeated | No data | pass | pass | pass | pass | pass |
| freeze-thaw | | | | | | |
| SEC-MALS- | <0.5% | 0 | / | / | / | / |
| repeated freeze- | aggregation | | | | | |
| thaw | | | | | | |
| Non-specific | −0.01-Pass | 0.022-Pass | 0.03-Pass | 0.038-Pass | 0.025-Pass | −0.080-Pass |
| interactions | | | | | | |
| Serum stability | Binding | Binding | Binding | Binding | Binding | Binding |
| | retained in all | retained in | retained in | retained in | retained in | retained in |
| | sera | all sera | all sera | all sera | all sera | all sera |
| No of aa | | 83 | 84 | 82 | 83 | 82 |
| identical to | | | | | | |
| germline | | | | | | |
| % ID with | | 87.4 | 88.4 | 86.3 | 87.4 | 86.3 |
| germline V- | | | | | | |
| segment* | | | | | | |

### Conclusion

The aim of this project was to humanize the MoTau01 antibody and to ensure the resulting antibody is capable of binding to TauC3 with comparable affinity when compared to the chimeric antibody. The MoTau01 antibody has been engineered and expressed as a humanized antibody without significant loss of binding affinity. The Tau01 HMKE, HMKM, HMKN, HMKO, HMKP humanized antibodies showed high affinities in binding ELISAs, Octet ranking and kinetic studies using Biacore, in the picomolar range (FIG. 31) and also pass all of our biophysical assays.

The Tau01 HMKP antibody shows the best drug-like characteristics as well as excellent kinetics of binding so was chosen as the lead candidate (Table 16). In our view the combination of the excellent binding, expression, thermostability, affinity and biophysical characterization properties make Tau01 HMKP a suitable candidate antibody for further development. Tau01 HMKN, HMKO and HMKE also exhibit excellent properties and are very good back up humanized lead candidates.

### Example 5 (Protocols)

The following protocols/procedures were used in Examples 1-4.

### RNeasy Mini Protocol for Isolation of Total RNA (Oiagen)

1. Disrupt cells by addition of Buffer RLT. For pelleted cells, loosen the cell pellet thoroughly by flicking the tube. Add Buffer RLT (600 l), and proceed to step 2. Note: Incomplete loosening of the cell pellet may lead to inefficient lysis and reduced yields.
2. Homogenize cells passing the lysate at least 5 times through an 18-20-gauge needle fitted to
an
RNase-free syringe.
3. Add 1 volume of 70% ethanol to the homogenized lysate, and mix thoroughly by pipetting. Do not centrifuge. The volume of lysate may be less than 600 μl due to loss during homogenization.
4. Transfer up to 700 μl of the sample, including any precipitate that may have formed, to an RNeasy spin column placed in a 2 mL collection tube. Close the lid gently, and centrifuge for 15 s at ≥8000×g. Discard the flow-through. Reuse the centrifuge tube in step 5.
5. Add 700 μl Buffer RW1 to the RNeasy column. Close the lid gently, and centrifuge for 15 s at ≥8000×g to wash the column membrane. Discard the flow-through. Reuse the centrifuge tube in step 6.
6. Add 500 μl Buffer RPE to the RNeasy column. Close the lid gently, and centrifuge for 15 s at ≥8000×g to wash the column membrane. Discard the flow-through. Reuse the centrifuge tube in step 7.
6. Add another 500 μl Buffer RPE to the RNeasy column. Close the lid gently, and centrifuge for 2 min at ≥8000×g to dry the RNeasy spin column membrane.
7. Place the RNeasy spin column in a new 2 mL collection tube and discard the old collection tube with the flow-through. Close the lid gently and centrifuge at full speed for 1 min.
8. To elute, transfer the RNeasy column to a new 1.5 mL collection tube. Add 30 μl of RNase-free water directly onto the RNeasy spin column membrane. Close the tube gently. Let it stand for 1 min, and then centrifuge for 1 min at ≥8000×g.

Protocol for 1^{st}-strand cDNA synthesis (GE Life Sciences)
1. Place the RNA sample in a microcentrifuge tube and add RNase-free water to bring the RNA to the appropriate volume (20 μL-12× dilution, see Table A).
2. Heat the RNA solution to 65° C. for 10 minutes, then chill on ice. Gently pipette the Bulk First-Strand cDNA Reaction Mix to obtain a uniform suspension. (Upon storage, the BSA may precipitate in the Mix; this precipitate will dissolve during incubation).
3. Add Bulk First-Strand cDNA Reaction Mix (11 μL) to a sterile 1.5 or 0.5 mL microcentrifuge tube. To this tube add 1 μL of DTT Solution, 1 μL (0.2 pg, 1:25 dilution) of NotI-d(T)18 primer and the heat-denatured RNA. Pipette up and down several times to mix.
4. Incubate at 37° C. for 1 hour and heat inactivate transcriptase for 5 min at 95° C.

TABLE A
Volumes of Components in First-Strand Reaction

| Bulk 1^{st} Strand | | | | Final Volume |
|---|---|---|---|---|
| Reaction Mix | Primer | DTT | RNA | First-Strand Reaction |
| 11 μL | 1 μL | 1 μL | 20 μL | 33 μL |

cDNA Purification
1. A simple protocol designed to remove contaminating First-Strand cDNA primer that could interfere with subsequent PCR reactions.
2. Add 99 μl of Buffer QG (from Qiagen Gel Extraction Kit, Cat. No: 28704) and 33 μl IPA. Mix and add to a QiaQuick Gel Extraction Column. Spin and discard the flow-through.
3. Wash the column once with 500 μl Buffer QG. Discard the flow-through.
4. Wash the column once with 750 μl Buffer PE. Discard the flow-through.
5. Spin the column to remove any residual alcohol and allow the column to dry.
6. Elute the cDNA with 50 μl distilled water pre-heated to 65° C.

### PCR Cloning of Mouse Variable Regions

1. Set up PCR reactions on the purified cDNA using the primers in Tables 1 and 2. Use a different forward primer in each reaction (MHV1-12 and 14 and MKV 1-11) as follows:

| *Kappa chain | *Heavy chain |
|---|---|
| 9 μl sterile water | 9 μl sterile water |
| 12.5 μl Phusion Flash | 12.5 μl Phusion Flash |
| Master Mix | Master Mix |
| 1.25 μl 10 μM MKCv2 primer | 1.25 μl 10 μM of MHCv2 primer mix |
| 1 μl 1st strand reaction | 1 μl 1st strand reaction |
| cDNA template. | cDNA template. |

*To each reaction add 1.25 μl of the appropriate MKV-v2 (Table 1) or MHV-v2 (Table 2) Forward primer

2. Cycle:

| | 3-step protocol | | | |
|---|---|---|---|---|
| Cycle step | Time | Temp. | | Cycles |
| Initial denaturation | 98° C. | 10 | s | 1 |
| Denaturation Temp. | 98° C. | 1 | s | 30 |
| Annealing | 60° C. | 5 | s | |
| Extension | 72° C. | 6-7 | s | |
| Final extension | 72° C. | 1 | min | 1 |

| | |
|---|---|
| 4° C. | hold |

3. Electrophorese a 5 μl sample from each PCR-reaction on a 2% (w/v) agarose gel to determine which of the leader primers produces a PCR-product. Positive PCR-clones will be about 420-500 bp in size.
4. For the positive clones, PCR purify the remaining sample using the QIAGEN PCR Purification Kit, eluting into 40 μL nuclease-free water. Send to an outside contractor (e.g. GATC) for PCR-fragment sequencing using the M13 Forward and M13 Reverse primers.

### QIAquick PCR Purification Microcentrifuge and Vacuum Protocol (QIAGEN)

1. All centrifugation steps are at 17,900×g (13,000 rpm) in a conventional table top microcentrifuge.
2. Add 5 volumes of Buffer PB to 1 volume of the PCR reaction and mix. If the colour of the mixture is orange or violet, add 10 μl of 3 M sodium acetate, pH 5.0, and mix. The colour of the mixture will turn yellow.
3. Place a QIAquick column in a provided 2 mL collection tube or into a vacuum manifold.
4. To bind DNA, apply the sample to the QIAquick column and centrifuge for 30-60 s or apply vacuum to the manifold until all samples have passed through the column. Discard flow-through and place the QIAquick column back into the same tube.
5. To wash, add 0.75 mL Buffer PE to the QIAquick column and centrifuge for 30-60 s or apply vacuum. Discard flow-through and place the QIAquick column back in the same tube.
6. Centrifuge the column in a 2 mL collection tube (provided) for 1 min.
7. Place each QIAquick column in a clean 1.5 mL microcentrifuge tube.
8. To elute DNA, add 40-50 μl Buffer EB (10 mM Tris-Cl, pH 8.5) to the centre of the QIAquick membrane and centrifuge the column for 1 min.

### Generation of mAb Expression Vectors by LIC

### Insert Preparation

1. Use sequence to generate LIC primers.
2. Perform LIC PCR on codon optimised, synthesised genes (Genscript) using LIC primers (Table 4).
3. Set up PCR reactions:

| | Volume ( for 20 μl |
|---|---|
| Reagent | final reaction volume) |
| H₂O | add to 50 μl |
| 2× Phusion PCR Master | 25 μl |
| Mix | |
| Primer Rev | 2.5 μl, Cf = 0.5 μM |
| Primer For | 2.5 μl, Cf = 0.5 μM |
| DNA | 1 μl |

Note:
Polymerase that generates blunt ended PCR products must be used in this step.
Other Polymerases which produce T overhangs are not suitable.

4. Cycle:

| | 3-step protocol | | | |
|---|---|---|---|---|
| Cycle step | Time | Temp. | | Cycles |
| Initial denaturation | 98° C. | 10 | s | 1 |
| Denaturation Temp. | 98° C. | 1 | s | 30 |
| Annealing | 72° C. | 5 | s | |
| Extension | 72° C. | 15 | s/1 kb | |
| Final extension | 72° C. | 1 | min | 1 |

| | |
|---|---|
| 4° C. | hold |

5. Run 5 μL of PCR products on a gel to ensure correct sized product—should be around 370 bp.
6. PCR purify products using Qiagen PCR purification kit to remove nucleotides and primers. Elute into 40 μL nuclease-free water.
7. T4 DNA Polymerase treat inserts:
   PCR product 40 μL
   10×NEB 2 4.5 μL
   dTTP (100 mM) NEB 1.25 μL
   T4 DNA Polymerase NEB 1 μL
8. Incubate at RT for 30 min and then inactivate enzyme at 70° C. for 20 min.

### Vector Preparation

9. Digest the LIC vectors (vector maps shown in FIGS. 3 and 4) with BfuAI by incubating at 50° C. for 3 hours or overnight:
   10×NEB buffer 3 10 μL
   100×BSA 1 μL
   BfuA1 5 μL
   LIC vector 5 μg
   dH₂0 to 100 μL
10. Following BfuAI digestion add: 2 μL of Bam HI and incubate at 37° C. for 2 hours.
11. Run the digested vector on a 1% (w/v) agarose/1×TAE gel containing 1×SYBR Safe DNA Stain. Two bands may be visible—cut out the higher MW band and extract using gel extraction kit, elute in 50 μL of EB.
12. T4 DNA polymerase treat the vector as follows:
   10×NEB buffer 2 6 μL
   100 mM dATP 1.5 μL
   T4 DNA Pol 1 μL
   BfuA1 digested vector 50 μL
13. Incubate at RT for 30 min and then inactivate the enzyme at 70° C. for 20 min.

### Cloning

14. Mix 1 μL of insert with 0.5 μL vector in a total of 10 μL nuclease-free water for 20 min RT. Always perform vector alone transformation.
15. Use the ligation mix to transform 25-50 μL of chemically competent Invitrogen TOP 10 bacteria following the manufacturer's instructions and spread on 90 mm diameter LB agar plates containing Kanamycin (50 μg/mL). Incubate overnight at 37° C.

### Pick Colonies from Transformation

16. PCR confirm using Phusion PCR Master Mix:

| | Volume (for 20 μl final reaction | |
|---|---|---|
| Reagent | volume) | |
| 2× Phusion PCR Master | 25 | μL |
| Mix | | |
| HCMVi primer | 1 | μL |
| HuG4/HuK LIC primer | 1 | μL |
| dH₂0 | to 23 | μL |

| | |
|---|---|
| DNA | Dip of colony (grow day culture |
| | of same colony) |

| | 3-step protocol | | | |
|---|---|---|---|---|
| Cycle step | Time | Temp. | | Cycles |
| Initial | 98° C. | 30 | s | 1 |
| denaturation | | | | |
| Denaturation | 98° C. | 30 | s | 25-30 |
| Temp. | | | | |
| Annealing | 65° C. | 5 | s | |
| Extension | 72° C. | 15 s /1 kb | | |
| Final extension | 72° C. | 5 | min | 1 |

| | |
|---|---|
| 4° C. | hold |

17. Run each PCR-reaction on a 2% agarose e-gel cassette and run for 15 min to determine the size of any PCR-product bands on the gel.
18. Grow starter cultures overnight using LB supplemented with Kanamycin to miniprep constructs and sequence the DNA (using the same primers) from at least two separate positive clones of the variable genes to identify any possible errors due to the PCR-reaction itself.
   Transformation of TOP10™ E. coli (Invitrogen Protocol)
1. Centrifuge the vial(s) containing the ligation reaction(s) briefly and place on ice.
2. Thaw, on ice, one 50 μL vial of One Shot cells for one or two ligations/transformations.
3. Pipet 1 to 2 μL of each ligation reaction directly into the vial of competent cells and mix by tapping gently. Do not mix by pipetting up and down. The remaining ligation mixture(s) can be stored at −20° C.
4. Incubate the vial(s) on ice for 15-30 minutes.
5. Incubate for exactly 30 seconds in the 42° C. water bath then place on ice for 2 min.
6. Add 250 μL of pre-warmed S.O.C medium to each vial.
7. Shake the vial(s) at 37° C. for exactly 1 hour at 225 rpm in a shaking incubator.
8. Spread 200 μL from each transformation vial on separate, labeled LB agar plates containing 500 μg/mL kanamycin.
9. Invert the plate(s) and incubate at 37° C. overnight.

### Plasmid DNA Miniprep Isolation Using QIAprep® (Qiagen Protocol)

1. Resuspend pelleted bacterial cells in 250 μL Buffer P1 and transfer to a microcentrifuge tube. Ensure that RNase A has been added to Buffer PL.
2. Add 250 μL Buffer P2 and invert the tube gently 4-6 times to mix.
3. Add 350 μL Buffer N3 and invert the tube immediately but gently 4-6 times. The solution should become cloudy.
4. Centrifuge for 10 min at 13,000 rpm (˜17,900×g) in a table-top microcentrifuge. A compact white pellet will form.
5. Apply the supernatant from step 4 to the QIAprep Spin Column by pipetting.
6. Centrifuge for 30-60 s. Discard the flow-through.
7. Wash column by adding 0.5 mL of Buffer PB and centrifuging for 30-60 s.
8. Wash column by adding 0.75 mL Buffer PE and centrifuging for 30-60 s.
9. Discard the flow-through and centrifuge for an additional 1 min.
10. Place the QIAprep column in a clean 1.5 mL microcentrifuge tube. To elute DNA, add 50 μL nuclease-free water to the center of the QIAprep Spin Column, let stand for 1 min, and centrifuge for 1 min.

### ExpiCHO Transfection in 24-Well Plates 1 ml Transfection (the ExpiCHO™ Expression System Kit—Invitrogen)

1. Subculture and expand expiCHO cells until the cells reach a density of approximately 4-6×10⁶ viable cells/mL.
2. On the day prior to transfection (Day −1), split the expiCHO culture to a final density of 3-4×10⁶ viable cells/mL and allow the cells to grow overnight.
3. Dilute cells to 6×10⁶ viable cells/mL.
4. Aliquot 0.9 mL of cells into each well of the 24-well plate to be used for transfection.
5. Prepare ExpiFectamine/DNA complexes.
6. Dilute plasmid DNA by adding 1 μL DNA to a final volume of 50 μL OptiPro for each well to be transfected (1 ug of plasmid DNA per mL of culture volume to be transfected).
7. Dilute 4 μL ExpiFectamine CHO reagent in 46 μL OptiPro medium for each well to be transfected (no incubation time required).
8. Add the diluted ExpiFectamine CHO to the diluted DNA and mix by gentle pipetting 3-4 times (incubation 1 to 5 min).
9. Add 100 μL of the complexation mixture to each well containing culture in the 24-well plate.
10. Cover the plates with a gas-permeable lid.
11. Incubate the 24-well plate in a 37° C. incubator with 8% C02 on an orbital shaker (recommended shake speed 225 rpm for shakers with a 19 mm orbital throw).
12. Add ExpiFectamine Enhancer (6 ul ExpiCHO enhancer) and ExpiCHO Feed (190 ul ExpiCHO feed) 18-22 hours post-transfection
13. Protein expression is typically complete and supernatant ready to be harvested by Day 7-8 post transfection.

### IgG Quantitation by Octet

1. Prepare 100 μl of each concentration of standard curve and ExpiCHO supernatant as follows:
   a. HuG4K Isotype standard at 500, 250, 125, 62.5, 31.25, 15.6, 7.81, 3.9 μg/ml, using ExpiCHO expression medium as a diluent
   b. Test (unknown) samples
2. Pre-soak (≥10 mins) protein G-coated biosensors (Pall ForteBio) in 200 μl ExpiCHO expression medium.
3. Aliquot 45 μl standards and test samples into a 384-well tilted-bottom plate in duplicate, including a media only control. Seal plate and spin (1000 rpm for 1 min) in bench-top centrifuge.
4. Remove plate seal and insert plate and presoaked sensors into Octet.
5. Perform quantitation as follows:
   a. Regenerate protein G-coated sensors in 10 mM glycine, pH1.5 for 5 seconds and neutralize in ExpiCHO expression medium for 5 seconds. Repeat three times.
   b. Measure standard or sample for 120 seconds.
   c. Repeat regeneration and neutralization step as above.
6. Import data into analysis software and fit data to a dose response-5 PL weighted fit to give IgG concentrations in pg/ml.

### TauC3 Binding ELISA

1. Coat each well of a 94/384-well MaxiSorp plate with 50/30 μL aliquots of 1 μg/mL of TauC3 in PBS per well in a 96/384-well plate respectively. Incubate overnight at 4° C.
2. Wash 3× with PBS-T (0.1% Tween20).
3. Block with 150/80 μL of PBS+5% BSA+0.1% Tween 20 per well in a 96/384-well plate respectively.
4. Incubate at 37° C. for 1 hour. Wash 3× with PBS-T (0.1% Tween20).
5. Add 50/30 μl of primary antibody serially diluted in PBS+0.2% BSA+0.1% Tween 20 to the assay plate (96/384-well plate respectively). Use a 3-fold dilution series starting from ˜4 μg/mL. Repeat the incubation and washing step (step 4).
6. Dilute the anti-human kappa chain HRP (Sigma A7164-1 mL) 3 ul per 10 ml in PBS+0.2% BSA+0.1% Tween 20 and add 50/30 μL to each well in a 96/384-well plate respectively. Repeat the incubation and washing step (step 4).
7. Add 75/20 μL of K-Blue substrate (Neogen) per well and incubate for 5-10 minutes at RT.
8. Stop the reaction by adding 50/10 μl of RED STOP solution (Neogen) to each well in a 96/384-well plate respectively.
9. Read the optical density at 650 nm using the Pherastar Plus.

### Tau01 Variant Screening and Affinity Determination by Octet

1. Immediately prior to use, incubate protein G-coated sensors (Pall ForteBio) in HBS-P⁺ buffer for 10 min.
2. Load 1 μg/mL antibody in HBS-P⁺ onto Protein G sensors for 600 seconds (immobilisation level of 0.8-1 nm).
3. Allow sensors to equilibrate in HBS-P⁺ for 180 seconds.
4. For screening assay, perform association step using 10 nM TauC3 for 600 seconds. For kinetics assay, load ˜0.5 μg/mL antibody in HBS-P⁺ for 10 mins and perform association step using a concentration range from 20 nM to 0.31 nM TauC3 for 10 mins.
5. Perform dissociation step in HBS-P⁺ for 600 seconds.
6. Regenerate sensors with 10 mM glycine, pH1.5-2.0 for 5-30 seconds and neutralise by incubating in HBS-P⁺ buffer for 30-60 seconds. Repeat three times.

### QuikChange Lightning Site-Directed Mutagenesis Kit (Stratagene)

1. Prepare the reaction(s) as indicated below:
   a. 5 μL of 10× reaction buffer
   b. 0.12 μL (25 ng) of RHA or RKA template
   c. 1.3 μL (125 ng) of oligonucleotide mutation primer For
   d. 1.3 μL (125 ng) of oligonucleotide mutation primer Rev
   e. 1 μL of dNTP mix
   f. 1.5 μL of QuikSolution reagent
   g. ddH2O to a final volume of 50 μL
   h. 1 μL of QuikChange Lightning Enzyme.
2. Cycle each reaction using the cycling parameters outlined in the following table:

| | 3-step protocol | | | |
|---|---|---|---|---|
| Cycle step | Time | Temp. | | Cycles |
| Initial denaturation | 95° C. | 2 | min | 1 |
| Denaturation Temp. | 95° C. | 20 | s | 18 |
| Annealing | 60° C. | 10 | s | |
| Extension | 68° C. | 3 | min | |
| Final extension | 68° C. | 5 | min | 1 |

| | |
|---|---|
| 4° C. | hold |

3. Add 2 μL of the Dpn I restriction enzyme
4. Gently and thoroughly mix each reaction, microcentrifuge briefly, then immediately incubate at 37° C. for 5 min to digest the parental dsDNA
5. Transform 2 μL of the Dpn I-treated DNA from each reaction into separate 45-μL (+2 μL j-ME) aliquots of XL10-Gold ultracompetent cells (see Transformation of TOP10™ E. coli).
6. Screen colonies using the Phusion method, miniprep and sequence to check for the correct mutation.

### Oiagen HiSpeed Maxiprep System Protocol

1. Pick from a freshly streaked selective plate or a glycerol stock of the clone of interest and inoculate a starter culture of 2-5 ml LB media supplemented with Kanamycin. Incubate for ˜8 hours at 37° C. with 250-300 rpm shaking.
2. Dilute the starter culture 1/1000 and inoculate 150-250 mL of LB media supplemented with Kanamycin from the starter culture and incubate overnight at 37° C. with 250-300 rpm shaking (12-16 hours).
3. Harvest the cells at 6,000×g for 15 mins. Discard the supernatant.
4. Resuspend cell pellet thoroughly in 10 mL of Buffer P1 by vortexing or pipetting.
5. Add 10 mL of Buffer P2. Invert vigorously 4-6 times to mix. Incubate for 5 min at room temperature.
6. Add 10 mL of chilled Buffer P3. Invert vigorously 4-6 times to mix.
7. Pour lysate into the barrel of a QIAfilter Cartridge. Incubate at room temperature for 10 min.
8. Equilibrate a HiSpeed Maxi Tip by applying 10 ml Buffer QBT and allow to empty by gravity flow.
9. Using a QIAfilter, filter the lysate into the equilibrated HiSpeed Maxi Tip. Allow the lysate to enter the resin by gravity flow.
10. Wash the HiSpeed Maxi Tip with 60 ml Buffer QC.
11. Elute DNA with 15 ml Buffer QF
12. Precipitate DNA by adding 10.5 ml isopropanol to the eluted DNA. Mix and incubate at room temperature for 5 min.
13. Transfer the eluate/isopropanol mixture into a 30 ml syringe and filter through the QIAprecipitator module.
14. Wash the DNA in the QIAprecipitator with 2 ml 70% Ethanol. Dry the membrane by pressing air through the QIAprecipitator numerous times.
15. Using a 5 ml syringe, elute the DNA in 1 ml nuclease-free water. Transfer the eluate into the syringe and elute for a second time.

### Thermal Stability Comparison

1. Dilute fully humanized antibodies and the chimeric control to 1 μg/mL in PBS/0.2% Tween and aliquot at the appropriate volume for the EC80 concentration into PCR tubes. Bring the volume up to 100 1 with the same buffer.
2. Heat separately each tube for 10 min at temperatures between 30° C. and 85° C. with a 5° C. interval and cool to 4° C.
3. Freeze down the 1 μg/mL stock for 1 h and then dilute to the EC80 concentration.
4. Perform the binding assay against TauC3 (Section 8.11) using 100 μl of each antibody per well (assay each temperature in duplicate) in a 96-well plate.

### Biacore Off-Rate Ranking and Kinetic Studies of the Tau01 Humanized Antibodies

### Off-Rate Ranking

1. Amine couple 0.5 μg/mL Human TauC3 in Acetate buffer pH 5 on 1 flow channel in a CM5 chip (GE Healthcare). Use the immobilization wizard to aim for ˜15 RU with HBS-EP+ as the running buffer.
2. Load antibody supernatants at 2.5 nM HBS-EP+ buffer at a flow rate of 30 μL/min for 300 s followed by 600 s dissociation and a 30 s regeneration using 3M MgCl₂. Export the raw data, subtract the buffer baseline and fit the data using a one-phase decay or two-phase decay in GraphPad Prism.

### Kinetics

1. Amine couple 0.5 μg/mL Human TauC3 in Acetate buffer pH 5 on 1 flow channel in a CM5 chip. Use the immobilization wizard to aim for ˜15 RU with HBS-EP+as the running buffer.
2. Dilute each antibody to 5 nM and produce a 2-fold dilution series down to 0.08 nM in HBS-EP+ buffer. Inject each concentration at 30 μL/min for 300 s, followed by a dissociation of 600 s, a 30 s regeneration using 3M MgCl₂ and a stabilization period of 600 s between cycles. Fit the data using a 1:1 Global fit.

### Binding Test FL Tau

1. Load 0.25 μg/mL antibody in HBS-EP+ buffer on a Protein G chip (GE Healthcare) at 10 μL/min for 30 s. Increase the flow rate to 30 μL/min and add 250 nM FL Tau in HBS-EP+ buffer for 180 s. Regenerate using 10 mM Glycine pH 1.5 for 30 s

### Purification of Antibody Candidates

Instrument: GE Healthcare ÄKTAxpress™ Purification System
Software: UNICORN
Columns: HiTrap MabSelect SuRe, 1 mL; HiLoad 16/600 Superdex 200 μg
Mobile phase: IgG Elution Buffer; Dulbecco's 1×PBS
Sample prep: Filtering through 0.22 μm
Injection volume: 200 mL Expi293 conditioned medium (1:1) in DPBS
Flow rate: Sample loading at 0.5 mL/min; Gel filtration at 1.5 mL/min; Elution at 1 mL/min

### SEC-MALS

1. 10 μl of each sample (1 mg/mL) was injected onto a SEC column (AdvanceBio SEC 300 Å, 4.6×150 mm, 2.7 μm, LC column, Agilent) and subsequently detected by three in-series detectors:
   a. UV (Agilent 1260 Infinity HPLC system with thermostated column compartment)
   b. Light-scattering (Wyatt Technology DAWN HELEOS)
   c. Differential Refractometer (Wyatt Technology Optilab TRex)
2. A constant flow-rate of 0.4 mL/min was applied using a mobile phase of Gibco's PBS (ThermoFisher) containing 0.05% sodium azide. All experiments were carried out at 25° C.
3. The data was analyzed with Wyatt Technology ASTRA software (version 6.1.2.83) and with the refractive index increment (dn/dc) set to 0.185 (i.e. for protein analysis).
4. All samples were stored at 4° C., prior to analysis by SEC-MALS.

### Dynamic Light Scattering (DLS)

1. Prepare 50 μl samples at 1.3 mg/ml (in Dulbecco's PBS; Sigma D8537) and aliquot into a 384-well polypropylene plate (Greiner bio-one).
2. Data is recorded on a Zetasizer APS (Malvern). All values were recorded in triplicate and processed using the associated Zetasizer software (version 7.11).
3. A cumulants analysis was performed to obtain mean particle size (z-average) and the polydispersity index (PDI).

### Mass Spectrometry

Mass spectrometry of purified chimeric and humanized candidate antibodies is depicted in FIG. 30.

### Thermal Shift Comparison

1. Prepare samples directly into 96 well white PCR plate in a final volume of 25 μL (purified antibody final concentration of 1 and 2 M).
2. Sypro Orange—make stock 1:100 in PBS buffer, then add 1:10 to final samples (e.g. 2.5 μL in 25 μL)
3. Load into the qPCR machine and use the MxPro software, SYBR Green method, (filter=FRROX, no reference dye). Thermal profile setup—71 cycles of 1° increase
4. Plot the results and determine Tm.

### Cross-Interaction Chromatography (CIC)

1. Samples were analyzed by two separate 20 1l injections (0.5 mg/mL); firstly onto a 1 mL NHS activated resin (GE Healthcare) coupled with 30 mg human polyclonal IgG (Sigma 14506) and secondly onto a 1 mL NHS activated resin blank coupled, as control column.
2. The mobile phase consisted of Dulbecco's PBS (Sigma D8537) containing 0.01% sodium azide (0.1 mL/min) and all experiments were performed at 25° C.
3. Eluted samples were detected by UV absorbance (Agilent 1260 Infinity HPLC system with thermostated column compartment) and data was analyzed using Wyatt Technology ASTRA software (version 6.1.2.83) to determine sample peak retention times. These were then used to calculate a retention factor k′:

$${k}^{′} = \frac{( Tr - Tm )}{Tm}$$

where Tᵣ is the retention time of the sample on the poly-IgG column and Tₘ is the retention time on the mock (control) column.

### Solubility

Load a Vivapore solvent absorption concentrator 7500 kDa MWCO (VP0502 Satorius) with 3.5-5.0 ml antibody solution at 1 mg/ml in PBS.
1. Monitor antibody concentration every 10 min by sampling a small amount for measuring on the Nanodrop 2000 (ε=1.4) and continue until the concentrated volume reaches the dead volume of ˜30-50 μl.
2. Plot the concentration values (mg/ml) against the corresponding time points to generate the concentration profiles.

### Circular Dichroism

1. Prepare 30 μL of sample, at 1 mg/ml (in Dulbecco's PBS; Sigma D8537).
2. Dilute the 1 mg/mL sample to 0.15× with 10 mM Phosphate buffer.
3. Readings were taken in a 1 mM spectrosil cuvette. Readings were taken with a DIT of 4 seconds, and a scanning speed of 20 nm/min, with a step size of 1 nm.
4. The averaged blank spectra was subtracted from the sample spectra, and then spectra converted to Δε. Spectra were then zeroed against their 256-260 nm values. Smoothing was performed by savitsky-golay filter via a custom excel function, sgFilter( ) using a quadratic polynomial with a a window size of 7 (−2, 3, 6, 7, 6, 3, −2). Spectra are shown with error bars, which are the average of the standard deviation at wavelengths +/−2 nm.
   pI Analysis Using cIEF
1. Samples were concentrated to >5 mg/ml and desalted to <50 mM NaCl levels whereupon 10 1 was added to 240 1 of a pharmalyte/urea gel mastermix that contains the pI markers of 4.5/5.1/9.5 and 10.
2. The sample was mixed for at least 5 min and then 200 μl was added to the sample PCR vial.
3. Samples were loaded onto the PA800 sample block along with the cIEF gel, catholyte, anolyte and chemical mobilizer rinse buffers into the chemical buffer block. The PA800 was loaded with a neutral capillary and the default “condition” method is run to prepare the capillary for sample analysis.
4. Each sample was run using the correct “separation” method that is dependant on the level of Urea present within the sample.
5. Data is analysed using 32 Karat software and the pI markers provide the standard curve to quantify the sample peak pI values.

### Antibody Serum Stability Assessment

1. Prepare 600 μl of polished antibody at 0.4 mg/mL in PBS.
2. Use Mouse serum (SCD-808), Human serum (S-123) and Cyno serum (S-118) from Seralab. Aliquot 150 ul serum and PBS control in a round bottom 96-well plate and add 50 ul 0.4 mg/mL antibody solution in PBS (final concentration of 100 ug/mL) in triplicates to each serum type in a tissue culture cabinet (BSL-2). Keep some at 4° C. to use as a control.

Serum incubation plate layout

| | | | |
|---|---|---|---|
| PBS | Mouse serum | Human serum | Cyno serum |
| PBS | Mouse serum | Human serum | Cyno serum |
| PBS | Mouse serum | Human serum | Cyno serum |

3. Seal the plate and incubate at 37° C.
4. Take 20 ul samples at specific intervals (e.g. day 10, day 20) under sterile conditions (BSL-2) to avoid contamination. Freeze at −20° C. until analysis.
5. Analyze the longest incubation first. Dilute samples appropriately and assay for antigen binding to TauC3 by generating ELISA binding curves for each sample (3*dilutions) (section 8.11). Compare the PBS/all serums per mAb sample on the same plate, using non-incubated antibody as control (NI).

All references, publications and patent documents cited herein, as well as text appearing in the figures and sequence listing, are hereby incorporated by reference in their entirety for all purposes to the same extent as if each were so individually denoted.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

What is claimed is:

1. An isolated anti-tauC3 antibody, which has have a sub-nanomolar specificity for TauC3 and as is at least 100 times more specific for TauC3 than for full length Tau, wherein the antibody comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, the variable heavy chain (V_{H}) polypeptide possessing at least 70% sequence identity to SEQ ID NO: 13; and (b) a variable light chain (V_{L}) polypeptide comprising CDR1 represented by SEQ ID NO: 10, CDR2 represented by SEQ ID NO: 11, and CDR3 represented by SEQ ID NO: 12, the variable light chain (V_{L}) polypeptide possessing at least 70% sequence identity to SEQ ID NO: 14, SEQ ID NO: 15, SEQ ID NO: 16, SEQ ID NO: 17, or SEQ ID NO: 18; and is a humanized antibody or a chimeric antibody.

2. The anti-TauC3 antibody of claim 1, which has a binding affinity (KD) for TauC3 from 1×10⁻¹⁰ to 1×10⁻¹² M.

3. The anti-TauC3 antibody of claim 1, wherein the off-rate Kd for TauC3 is from 1×10⁻⁴ to 1×10⁻³ S-1.

4. The anti-TauC3 antibody of claim 1, wherein the variable heavy chain (V_{H}) polypeptide comprises SEQ ID NO: 13; and the variable light chain (V_{L}) polypeptide comprises a sequence selected from the group consisting of SEQ ID NO: 14, SEQ ID NO: 15, SEQ ID NO: 16, SEQ ID NO: 17, and SEQ ID NO: 18.

5. The anti-TauC3 antibody of claim 1, wherein, on the variable heavy chain (V_{H}) polypeptide, CDR1 is a polypeptide of SEQ ID NO: 7, CDR2 is a polypeptide of SEQ ID NO: 8, and CDR3 is a polypeptide of SEQ ID NO: 9; and on the light chain (V_{L}) polypeptide, CDR1 is a polypeptide of SEQ ID NO: 10, CDR2 is a polypeptide of SEQ ID NO: 11, and CDR3 is a polypeptide of SEQ ID NO: 12.

6. The anti-TauC3 antibody of claim 5, wherein the variable heavy chain (V_{H}) polypeptide is a polypeptide of SEQ ID NO: 13 and the variable light chain (V_{L}) polypeptide is a polypeptide of SEQ ID NO: 14, SEQ ID NO: 15, SEQ ID NO: 16, SEQ ID NO: 17, or SEQ ID NO: 18.

7. The anti-TauC3 antibody of claim 1, which comprises a V_{H} chain polypeptide possessing at least 90% sequence identity to SEQ ID NO: 13, and a V_{L} chain polypeptide possessing at least 90% sequence identity to SEQ ID NO: 14, SEQ ID NO: 15, SEQ ID NO: 16, SEQ ID NO: 17, or SEQ ID NO: 18.

8. The anti-TauC3 antibody of claim 1, which comprises a V_{H} chain polypeptide possessing at least 95% sequence identity to SEQ ID NO: 13, and a V_{L} chain polypeptide possessing at least 95% sequence identity to SEQ ID NO: 14, SEQ ID NO: 15, SEQ ID NO: 16, SEQ ID NO: 17, or SEQ ID NO: 18.

9. The anti-TauC3 antibody of claim 1, which is a humanized antibody.

10. The anti-TauC3 antibody of claim 1, which is a chimeric antibody.

11. The anti-TauC3 antibody of claim 1, which is a humanized antibody having a binding affinity (KD) for TauC3 from about 10 pM to about 40 pM.

12. The anti-TauC3 antibody of claim 11, which has a binding affinity (KD) for TauC3 from about 10 to about 35 pM.

13. A method of treating a tauopathy in a subject comprising administering a therapeutically effective amount of the anti-TauC3 antibody of claim 1 to a subject in need thereof.

14. The method of claim 13, wherein the tauopathy is selected from the group consisting of Alzheimer disease, progressive supranuclear palsy, frontotemporal dementia, traumatic brain injury, Pick's disease, corticobasal degeneration, and frontotemporal lobar degeneration.

15. The method of claim 14, wherein the tauopathy is Alzheimer disease.

16. The method of claim 13, wherein the anti-TauC3 antibody is a humanized antibody.

17. The method of claim 16, wherein the anti-TauC3 antibody has a binding affinity (KD) for TauC3 from about 10 pM to about 40 pM.

18. A pharmaceutical composition comprising an anti-TauC3 antibody according to claim 1 and one or more pharmaceutically acceptable excipient(s).

19. The pharmaceutical composition of claim 18, wherein the anti-TauC3 comprises (a) a variable heavy chain (V_{H}) polypeptide comprising CDR1 represented by SEQ ID NO: 7, CDR2 represented by SEQ ID NO: 8, and CDR3 represented by SEQ ID NO: 9, and (b) a variable light chain (V_{L}) polypeptide comprising CDR1 represented by Seq. ID No. 10, CDR2 represented by SEQ. ID NO: 11, and CDR3 represented by SEQ ID NO: 12.

20. The pharmaceutical composition of claim 19, wherein the anti-TauC3 antibody has an aqueous solubility of from about 50 mg/ml to about 200 mg/ml.


SEQUENCE LISTING (153 sequences)

SEQ ID NO: 1 - protein, 441 aa, synthetic construct
  REGION 1..441: note = htau 40
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 410 aa, synthetic construct
  REGION 1..410: note = Tau 2N3R
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSLDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPAHLSNVSSTGSIDMVDSPQLATLADEVSASLAKQGL

SEQ ID NO: 3 - protein, 412 aa, synthetic construct
  REGION 1..412: note = Tau IN4R
MAEPRQEFEVMEDHAGTYGLGDRKDQGGYTMHQDQEGDTDAGLKESPLQTPTGDGSEEPGSETSDAKSTPTAEAEEAGIGDTPSLEDEAAGHVTQARMVSKSLDGTGSDDKKAKGADGKTLIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSLDNILHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 383 aa, synthetic construct
  REGION 1..383: note = Tau ON4R
MAEPRQEFEVMEDHAGTYGLGDRLDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVATPPKSPSSAKSRLQTAPVPMPDLKNVKSLIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKALTDHGAEIVYKSPVVSGDTSPRHLSNVSSTGSIDMVDSPQLATLADEVSASLAKQGL

SEQ ID NO: 5 - protein, 381 aa, synthetic construct
  REGION 1..381: note = Tau 1N3R
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 381 aa, synthetic construct; identical to SEQ ID NO: 5
  REGION 1..381: note = Tau 0N3R

SEQ ID NO: 7 - protein, 8 aa, synthetic construct
  REGION 1..8: note = Heavy Chain CDR1
GFTFNTYA

SEQ ID NO: 8 - protein, 10 aa, synthetic construct
  REGION 1..10: note = Heavy Chain CDR2
IRSKSNNYAT

SEQ ID NO: 9 - protein, 6 aa, synthetic construct
  REGION 1..6: note = VGGGDF
VGGGDF

SEQ ID NO: 10 - protein, 6 aa, synthetic construct
  REGION 1..6: note = Light Chain CDR1
QEISVY

SEQ ID NO: 11 - skipped

SEQ ID NO: 12 - protein, 9 aa, synthetic construct
  REGION 1..9: note = LQYVRYPWT
LQYVRYPWT

SEQ ID NO: 13 - protein, 115 aa, synthetic construct
  REGION 1..115: note = Heavy Chain HM
LVQLVESGGGLVQPGGSLKLSCAASGFTFNTYAMNWVRQASGKGLEWVARIRSKSNNYATYYAASVKGRFTISRDDSKSMAYLQMDSLKTEDTAVYYCVGGGDFWGQGTLVTVSS

SEQ ID NO: 14 - protein, 107 aa, synthetic construct
  REGION 1..107: note = light Chain KE
DIQMTQSPSSLSASVGDRVTITCRASQEISVYLGWFQQKPGKAPKRLIYGAFKLQSGVPSRFSGSRSGTEFTLTISSLQPEDFATYYCLQYVRYPWTFGGGTKVEIK

SEQ ID NO: 15 - protein, 107 aa, synthetic construct
  REGION 1..107: note = light Chain KN
DIQMTQSPSSLSASVGDRVTITCRASQEISVYLGWYQQKPGKAPKRLIYGAFTLQSGVPSRFSGSRSGTEYTLTISSLQPEDFATYYCLQYVRYPWTFGGGTKVEIK

SEQ ID NO: 16 - protein, 107 aa, synthetic construct
  REGION 1..107: note = light Chain KO
DIQMTQSPSSLSASVGDRVTITCRASQEISVYLGWYQQKPGKAPKRLIYGAFSLQSGVPSRFSGSRSGTEYTLTISSLQPEDFATYYCLQYVRYPWTFGGGTKVEIK

SEQ ID NO: 17 - protein, 107 aa, synthetic construct
  REGION 1..107: note = light Chain KP
DIQMTQSPSSLSASVGDRVTITCRASQEISVYLGWFQQKPGKAPKRLIYGAFKLQSGVPSRFSGSRSGTEYTLTISSLQPEDFATYYCLQYVRYPWTFGGGTKVEIK

SEQ ID NO: 18 - protein, 107 aa, synthetic construct
  REGION 1..107: note = light Chain KM
DIQMTQSPSSLSASVGDRVTITCRASQEISVYLSWFQQKPGKAIKRLIYGAFSLQSGVPSRFSGSRSGTEYTLTISSLQPEDFATYYCLQYVRYPWTFGGGTKVEIK

SEQ ID NO: 19 - protein, 10 aa, synthetic construct
  REGION 1..10: note = immunizing peptide
SSTGSIDMVD

SEQ ID NO: 20 - DNA, 48 bp, synthetic construct
  misc_feature 1..48: note = primer
  source 1..48: mol_type = other DNA
tgtaaaacgacggccagtatgaagttgcctgttaggctgttggtgctg

SEQ ID NO: 21 - DNA, 48 bp, synthetic construct
  misc_feature 1..48: note = primer
  source 1..48: mol_type = other DNA
tgtaaaacgacggccagtatggagwcagacacactcctgytatgggtg

SEQ ID NO: 22 - DNA, 48 bp, synthetic construct
  misc_feature 1..48: note = primer
  source 1..48: mol_type = other DNA
tgtaaaacgacggccagtatgagtgtgctcactcaggtcctggsgttg

SEQ ID NO: 23 - DNA, 51 bp, synthetic construct
  misc_feature 1..51: note = primer
  source 1..51: mol_type = other DNA
tgtaaaacgacggccagtatgaggrcccctgctcagwttyttggmwtcttg

SEQ ID NO: 24 - DNA, 47 bp, synthetic construct
  misc_feature 1..47: note = primer
  source 1..47: mol_type = other DNA
tgtaaaacgacggccagtatggatttwaggtgcagattwtcagcttc

SEQ ID NO: 25 - DNA, 45 bp, synthetic construct
  misc_feature 1..45: note = primer
  source 1..45: mol_type = other DNA
tgtaaaacgacggccagtatgaggtkckktgktsagstsctgrgg

SEQ ID NO: 26 - DNA, 49 bp, synthetic construct
  misc_feature 1..49: note = primer
  source 1..49: mol_type = other DNA
tgtaaaacgacggccagtatgggcwtcaagatggagtcacakwyycwgg

SEQ ID NO: 27 - DNA, 49 bp, synthetic construct
  misc_feature 1..49: note = primer
  source 1..49: mol_type = other DNA
tgtaaaacgacggccagtatgtggggayctktttycmmtttttcaattg

SEQ ID NO: 28 - DNA, 43 bp, synthetic construct
  misc_feature 1..43: note = primer
  source 1..43: mol_type = other DNA
tgtaaaacgacggccagtatggtrtccwcasctcagttccttg

SEQ ID NO: 29 - DNA, 45 bp, synthetic construct
  misc_feature 1..45: note = primer
  source 1..45: mol_type = other DNA
tgtaaaacgacggccagtatgtatatatgtttgttgtctatttct

SEQ ID NO: 30 - DNA, 46 bp, synthetic construct
  misc_feature 1..46: note = primer
  source 1..46: mol_type = other DNA
tgtaaaacgacggccagtatggaagccccagctcagcttctcttcc

SEQ ID NO: 31 - DNA, 44 bp, synthetic construct
  misc_feature 1..44: note = primer
  source 1..44: mol_type = other DNA
tgtaaaacgacggccagtatgragtywcagacccaggtcttyrt

SEQ ID NO: 32 - DNA, 44 bp, synthetic construct
  misc_feature 1..44: note = primer
  source 1..44: mol_type = other DNA
tgtaaaacgacggccagtatggagacacattctcaggtctttgt

SEQ ID NO: 33 - DNA, 44 bp, synthetic construct
  misc_feature 1..44: note = primer
  source 1..44: mol_type = other DNA
tgtaaaacgacggccagtatggattcacaggcccaggttcttat

SEQ ID NO: 34 - DNA, 44 bp, synthetic construct
  misc_feature 1..44: note = primer
  source 1..44: mol_type = other DNA
tgtaaaacgacggccagtatgatgagtcctgcccagttcctgtt

SEQ ID NO: 35 - DNA, 47 bp, synthetic construct
  misc_feature 1..47: note = primer
  source 1..47: mol_type = other DNA
tgtaaaacgacggccagtatgaatttgcctgttcatctcttggtgct

SEQ ID NO: 36 - DNA, 47 bp, synthetic construct
  misc_feature 1..47: note = primer
  source 1..47: mol_type = other DNA
tgtaaaacgacggccagtatggattttcaattggtcctcatctcctt

SEQ ID NO: 37 - DNA, 44 bp, synthetic construct
  misc_feature 1..44: note = primer
  source 1..44: mol_type = other DNA
tgtaaaacgacggccagtatgaggtgcctarctsagttcctgrg

SEQ ID NO: 38 - DNA, 44 bp, synthetic construct
  misc_feature 1..44: note = primer
  source 1..44: mol_type = other DNA
tgtaaaacgacggccagtatgaagtactctgctcagtttctagg

SEQ ID NO: 39 - DNA, 44 bp, synthetic construct
  misc_feature 1..44: note = primer
  source 1..44: mol_type = other DNA
tgtaaaacgacggccagtatgaggcattctcttcaattcttggg

SEQ ID NO: 40 - DNA, 38 bp, synthetic construct
  misc_feature 1..38: note = primer
  source 1..38: mol_type = other DNA
caggaaacagctatgaccactggatggtgggaagatgg

SEQ ID NO: 41 - DNA, 45 bp, synthetic construct
  misc_feature 1..45: note = primer
  source 1..45: mol_type = other DNA
tgtaaaacgacggccagtatgaaatgcagctggggcatsttcttc

SEQ ID NO: 42 - DNA, 44 bp, synthetic construct
  misc_feature 1..44: note = primer
  source 1..44: mol_type = other DNA
tgtaaaacgacggccagtatgggatggagctrtatcatsytctt

SEQ ID NO: 43 - DNA, 45 bp, synthetic construct
  misc_feature 1..45: note = primer
  source 1..45: mol_type = other DNA
tgtaaaacgacggccagtatgaagwtgtggttaaactgggttttt

SEQ ID NO: 44 - DNA, 43 bp, synthetic construct
  misc_feature 1..43: note = primer
  source 1..43: mol_type = other DNA
tgtaaaacgacggccagtatgractttgggytcagcttgrttt

SEQ ID NO: 45 - DNA, 48 bp, synthetic construct
  misc_feature 1..48: note = primer
  source 1..48: mol_type = other DNA
tgtaaaacgacggccagtatggactccaggctcaatttagttttcctt

SEQ ID NO: 46 - DNA, 45 bp, synthetic construct
  misc_feature 1..45: note = primer
  source 1..45: mol_type = other DNA
tgtaaaacgacggccagtatggctgtcytrgsgctrctcttctgc

SEQ ID NO: 47 - DNA, 44 bp, synthetic construct
  misc_feature 1..44: note = primer
  source 1..44: mol_type = other DNA
tgtaaaacgacggccagtatggratggagckggrtctttmtctt

SEQ ID NO: 48 - DNA, 41 bp, synthetic construct
  misc_feature 1..41: note = primer
  source 1..41: mol_type = other DNA
tgtaaaacgacggccagtatgagagtgctgattcttttgtg

SEQ ID NO: 49 - DNA, 48 bp, synthetic construct
  misc_feature 1..48: note = primer
  source 1..48: mol_type = other DNA
tgtaaaacgacggccagtatggmttgggtgtggamcttgctattcctg

SEQ ID NO: 50 - DNA, 45 bp, synthetic construct
  misc_feature 1..45: note = primer
  source 1..45: mol_type = other DNA
tgtaaaacgacggccagtatgggcagacttacattctcattcctg

SEQ ID NO: 51 - DNA, 46 bp, synthetic construct
  misc_feature 1..46: note = primer
  source 1..46: mol_type = other DNA
tgtaaaacgacggccagtatggattttgggctgattttttttattg

SEQ ID NO: 52 - DNA, 45 bp, synthetic construct
  misc_feature 1..45: note = primer
  source 1..45: mol_type = other DNA
tgtaaaacgacggccagtatgatggtgttaagtcttctgtacctg

SEQ ID NO: 53 - DNA, 49 bp, synthetic construct
  misc_feature 1..49: note = primer
  source 1..49: mol_type = other DNA
tgtaaaacgacggccagtatgaacaggcttacttcctcattgctgctgc

SEQ ID NO: 54 - DNA, 39 bp, synthetic construct
  misc_feature 1..39: note = primer
  source 1..39: mol_type = other DNA
caggaaacagctatgacccagtggatagacagatggggg

SEQ ID NO: 55 - DNA, 39 bp, synthetic construct
  misc_feature 1..39: note = primer
  source 1..39: mol_type = other DNA
caggaaacagctatgacccagtggatagaccgatggggc

SEQ ID NO: 56 - DNA, 39 bp, synthetic construct
  misc_feature 1..39: note = primer
  source 1..39: mol_type = other DNA
caggaaacagctatgacccagtggatagactgatggggg

SEQ ID NO: 57 - DNA, 39 bp, synthetic construct
  misc_feature 1..39: note = primer
  source 1..39: mol_type = other DNA
caggaaacagctatgacccaagggatagacagatggggc

SEQ ID NO: 58 - DNA, 41 bp, synthetic construct
  misc_feature 1..41: note = primer
  source 1..41: mol_type = other DNA
tgtaaaacgacggccagtgaggtgcaggttgttgagtctgg

SEQ ID NO: 59 - DNA, 20 bp, synthetic construct
  misc_feature 1..20: note = primer
  source 1..20: mol_type = other DNA
tgttcctttccatgggtctt

SEQ ID NO: 60 - DNA, 21 bp, synthetic construct
  misc_feature 1..21: note = primer
  source 1..21: mol_type = other DNA
ctctcggaggtgctcctggag

SEQ ID NO: 61 - DNA, 20 bp, synthetic construct
  misc_feature 1..20: note = primer
  source 1..20: mol_type = other DNA
gcagttccagatttcaactg

SEQ ID NO: 62 - DNA, 18 bp, synthetic construct
  misc_feature 1..18: note = primer
  source 1..18: mol_type = other DNA
tgtaaaacgacggccagt

SEQ ID NO: 63 - DNA, 18 bp, synthetic construct
  misc_feature 1..18: note = primer
  source 1..18: mol_type = other DNA
caggaaacagctatgacc

SEQ ID NO: 64 - DNA, 45 bp, synthetic construct
  misc_feature 1..45: note = primer
  source 1..45: mol_type = other DNA
ctctggctccctgataccaccggagaggtgcaggtggtggagagc

SEQ ID NO: 65 - DNA, 45 bp, synthetic construct
  misc_feature 1..45: note = primer
  source 1..45: mol_type = other DNA
ctctggctccctgataccaccggagatatccagatgacacagtct

SEQ ID NO: 66 - DNA, 38 bp, synthetic construct
  misc_feature 1..38: note = primer
  source 1..38: mol_type = other DNA
gggcccttggtggaggcggagctcactgtcagggcggt

SEQ ID NO: 67 - DNA, 44 bp, synthetic construct
  misc_feature 1..44: note = primer
  source 1..44: mol_type = other DNA
cgcttggtgctgccacagttctcttgatctccagctttgtgccg

SEQ ID NO: 68 - DNA, 46 bp, synthetic construct
  misc_feature 1..46: note = primer
  source 1..46: mol_type = other DNA
ctctggctccctgataccaccggactggtgcagctggtggaaagcg

SEQ ID NO: 69 - DNA, 46 bp, synthetic construct
  misc_feature 1..46: note = primer
  source 1..46: mol_type = other DNA
ctctggctccctgataccaccggagaggtgcaggtggtggaaagcg

SEQ ID NO: 70 - DNA, 39 bp, synthetic construct
  misc_feature 1..39: note = primer
  source 1..39: mol_type = other DNA
gggcccttggtggaggcggagctcactgtcaccagggtg

SEQ ID NO: 71 - DNA, 30 bp, synthetic construct
  misc_feature 1..30: note = primer
  source 1..30: mol_type = other DNA
cctgataccaccggagaggtgcagctggtg

SEQ ID NO: 72 - DNA, 30 bp, synthetic construct
  misc_feature 1..30: note = primer
  source 1..30: mol_type = other DNA
caccagctgcacctctccggtggtatcagg

SEQ ID NO: 73 - DNA, 27 bp, synthetic construct
  misc_feature 1..27: note = primer
  source 1..27: mol_type = other DNA
cggactggtgcaggtggtggaaagcgg

SEQ ID NO: 74 - DNA, 27 bp, synthetic construct
  misc_feature 1..27: note = primer
  source 1..27: mol_type = other DNA
ccgctttccaccacctgcaccagtccg

SEQ ID NO: 75 - DNA, 34 bp, synthetic construct
  misc_feature 1..34: note = primer
  source 1..34: mol_type = other DNA
tttaacacatatgcaatgaactgggtgcggcagg

SEQ ID NO: 76 - DNA, 34 bp, synthetic construct
  misc_feature 1..34: note = primer
  source 1..34: mol_type = other DNA
cctgccgcacccagttcattgcatatgtgttaaa

SEQ ID NO: 77 - DNA, 27 bp, synthetic construct
  misc_feature 1..27: note = primer
  source 1..27: mol_type = other DNA
ctggagtgggtggcccggatcagatct

SEQ ID NO: 78 - DNA, 27 bp, synthetic construct
  misc_feature 1..27: note = primer
  source 1..27: mol_type = other DNA
agatctgatccgggccacccactccag

SEQ ID NO: 79 - DNA, 49 bp, synthetic construct
  misc_feature 1..49: note = primer
  source 1..49: mol_type = other DNA
ctaagagcaacaattatgcaacatattatgcagcatctgtgaagggcag

SEQ ID NO: 80 - DNA, 49 bp, synthetic construct
  misc_feature 1..49: note = primer
  source 1..49: mol_type = other DNA
ctgcccttcacagatgctgcataatatgttgcataattgttgctcttag

SEQ ID NO: 81 - DNA, 40 bp, synthetic construct
  misc_feature 1..40: note = primer
  source 1..40: mol_type = other DNA
tatgcaacagcatatgcagattctgtgaagggcaggttca

SEQ ID NO: 82 - DNA, 40 bp, synthetic construct
  misc_feature 1..40: note = primer
  source 1..40: mol_type = other DNA
tgaacctgcccttcacagaatctgcatatgctgttgcata

SEQ ID NO: 83 - DNA, 35 bp, synthetic construct
  misc_feature 1..35: note = primer
  source 1..35: mol_type = other DNA
ccgcgacgattctaagagtacagcctatctgcaga

SEQ ID NO: 84 - DNA, 35 bp, synthetic construct
  misc_feature 1..35: note = primer
  source 1..35: mol_type = other DNA
tctgcagataggctgtactcttagaatcgtcgcgg

SEQ ID NO: 85 - DNA, 40 bp, synthetic construct
  misc_feature 1..40: note = primer
  source 1..40: mol_type = other DNA
tctcccgcgacgattctaagaatatggcctatctgcagat

SEQ ID NO: 86 - DNA, 40 bp, synthetic construct
  misc_feature 1..40: note = primer
  source 1..40: mol_type = other DNA
atctgcagataggccatattcttagaatcgtcgcgggaga

SEQ ID NO: 87 - DNA, 39 bp, synthetic construct
  misc_feature 1..39: note = primer
  source 1..39: mol_type = other DNA
gacgattctaagaatacagtctatctgcagatggactcc

SEQ ID NO: 88 - DNA, 39 bp, synthetic construct
  misc_feature 1..39: note = primer
  source 1..39: mol_type = other DNA
ggagtccatctgcagatagactgtattcttagaatcgtc

SEQ ID NO: 89 - DNA, 46 bp, synthetic construct
  misc_feature 1..46: note = primer
  source 1..46: mol_type = other DNA
ctctggctccctgataccaccggagacatccagatgacccagtctc

SEQ ID NO: 90 - DNA, 46 bp, synthetic construct
  misc_feature 1..46: note = primer
  source 1..46: mol_type = other DNA
ctctggctccctgataccaccggagacatccagatgacacagtctc

SEQ ID NO: 91 - DNA, 44 bp, synthetic construct
  misc_feature 1..44: note = primer
  source 1..44: mol_type = other DNA
cgcttggtgctgccacagttctcttgatctccacctttgtgccg

SEQ ID NO: 92 - DNA, 33 bp, synthetic construct
  misc_feature 1..33: note = primer
  source 1..33: mol_type = other DNA
ttctgctgataccagctcaggtacacggagatc

SEQ ID NO: 93 - DNA, 29 bp, synthetic construct
  misc_feature 1..29: note = primer
  source 1..29: mol_type = other DNA
tgtacctgggctggtttcagcagaagccc

SEQ ID NO: 94 - DNA, 29 bp, synthetic construct
  misc_feature 1..29: note = primer
  source 1..29: mol_type = other DNA
gggcttctgctgaaaccagcccaggtaca

SEQ ID NO: 95 - DNA, 34 bp, synthetic construct
  misc_feature 1..34: note = primer
  source 1..34: mol_type = other DNA
gaagcccggcaaggccattaagcggctgatctac

SEQ ID NO: 96 - DNA, 34 bp, synthetic construct
  misc_feature 1..34: note = primer
  source 1..34: mol_type = other DNA
gtagatcagccgcttaatggccttgccgggcttc

SEQ ID NO: 97 - DNA, 28 bp, synthetic construct
  misc_feature 1..28: note = primer
  source 1..28: mol_type = other DNA
atctacggcgccttcacgctgcagtccg

SEQ ID NO: 98 - DNA, 28 bp, synthetic construct
  misc_feature 1..28: note = primer
  source 1..28: mol_type = other DNA
cggactgcagcgtgaaggcgccgtagat

SEQ ID NO: 99 - DNA, 31 bp, synthetic construct
  misc_feature 1..31: note = primer
  source 1..31: mol_type = other DNA
ggatccagatctggcagcgagtttaccctga

SEQ ID NO: 100 - DNA, 31 bp, synthetic construct
  misc_feature 1..31: note = primer
  source 1..31: mol_type = other DNA
tcagggtaaactcgctgccagatctggatcc

SEQ ID NO: 101 - DNA, 35 bp, synthetic construct
  misc_feature 1..35: note = primer
  source 1..35: mol_type = other DNA
cagatctggcaccgagtataccctgacaatctcta

SEQ ID NO: 102 - DNA, 35 bp, synthetic construct
  misc_feature 1..35: note = primer
  source 1..35: mol_type = other DNA
tagagattgtcagggtatactcggtgccagatctg

SEQ ID NO: 103 - DNA, 30 bp, synthetic construct
  misc_feature 1..30: note = primer
  source 1..30: mol_type = other DNA
ctacggcgccttcagcctgcagtccggagt

SEQ ID NO: 104 - DNA, 30 bp, synthetic construct
  misc_feature 1..30: note = primer
  source 1..30: mol_type = other DNA
actccggactgcaggctgaaggcgccgtag

SEQ ID NO: 105 - protein, 115 aa, synthetic construct
  REGION 1..115: note = heavy chain
EVQVVESGGGLVQPKGSLKLSCAASGFTFNTYAMNWVRQAPGKGLEWVARIRSKSNNYATYYADSVKDRFTISRDDSQSMVYLQMNNLKTEDTAMYYCVGGGDFWGQGTALTVSS

SEQ ID NO: 106 - protein, 114 aa, synthetic construct
  REGION 1..114: note = heavy chain
LVQLVESGGGLVQPGGSLKLSCAASGFTFSGSAMHWVRQASGKGLEWVGRIRSKANSYATAYAASVKGRFTISRDDSKNTAYLQMDSLKTEDTAVYYCTTYEGWGQGTLVTVSS

SEQ ID NO: 107 - protein, 115 aa, synthetic construct
  REGION 1..115: note = heavy chain
LVQLVESGGGLVQPGGSLKLSCAASGFTFNTYAMHWVRQASGKGLEWVGRIRSKSNNYATAYAASVKGRFTISRDDSKNTAYLQMDSLKTEDTAVYYCVGGGDFWGQGTLVTVSS

SEQ ID NO: 108 - protein, 115 aa, synthetic construct
  REGION 1..115: note = heavy chain
EVQVVESGGGLVQPGGSLKLSCAASGFTFNTYAMNWVRQASGKGLEWVARIRSKSNNYATYYADSVKGRFTISRDDSKSMVYLQMDSLKTEDTAVYYCVGGGDFWGQGTLVTVSS

SEQ ID NO: 109 - protein, 115 aa, synthetic construct
  REGION 1..115: note = heavy chain
LVQLVESGGGLVQPGGSLKLSCAASGFTFNTYAMNWVRQASGKGLEWVGRIRSKSNNYATYYAASVKGRFTISRDDSKSMAYLQMDSLKTEDTAVYYCVGGGDFWGQGTLVTVSS

SEQ ID NO: 110 - protein, 115 aa, synthetic construct
  REGION 1..115: note = heavy chain
EVQLVESGGGLVQPGGSLKLSCAASGFTFNTYAMHWVRQASGKGLEWVGRIRSKSNNYATAYAASVKGRFTISRDDSKNTAYLQMDSLKTEDTAVYYCVGGGDFWGQGTLVTVSS

SEQ ID NO: 111 - protein, 115 aa, synthetic construct
  REGION 1..115: note = heavy chain
LVQVVESGGGLVQPGGSLKLSCAASGFTFNTYAMHWVRQASGKGLEWVGRIRSKSNNYATAYAASVKGRFTISRDDSKNTAYLQMDSLKTEDTAVYYCVGGGDFWGQGTLVTVSS

SEQ ID NO: 112 - protein, 115 aa, synthetic construct
  REGION 1..115: note = heavy chain
LVQLVESGGGLVQPGGSLKLSCAASGFTFNTYAMNWVRQASGKGLEWVGRIRSKSNNYATAYAASVKGRFTISRDDSKNTAYLQMDSLKTEDTAVYYCVGGGDFWGQGTLVTVSS

SEQ ID NO: 113 - protein, 115 aa, synthetic construct
  REGION 1..115: note = heavy chain
LVQLVESGGGLVQPGGSLKLSCAASGFTFNTYAMHWVRQASGKGLEWVARIRSKSNNYATAYAASVKGRFTISRDDSKNTAYLQMDSLKTEDTAVYYCVGGGDFWGQGTLVTVSS

SEQ ID NO: 114 - protein, 115 aa, synthetic construct
  REGION 1..115: note = heany chain
LVQLVESGGGLVQPGGSLKLSCAASGFTFNTYAMHWVRQASGKGLEWVGRIRSKSNNYATYYAASVKGRFTISRDDSKNTAYLQMDSLKTEDTAVYYCVGGGDFWGQGTLVTVSS

SEQ ID NO: 115 - protein, 115 aa, synthetic construct
  REGION 1..115: note = heavy chain
LVQLVESGGGLVQPGGSLKLSCAASGFTFNTYAMHWVRQASGKGLEWVGRIRSKSNNYATAYADSVKGRFTISRDDSKNTAYLQMDSLKTEDTAVYYCVGGGDFWGQGTLVTVSS

SEQ ID NO: 116 - protein, 115 aa, synthetic construct
  REGION 1..115: note = heavy chain
LVQLVESGGGLVQPGGSLKLSCAASGFTFNTYAMHWVRQASGKGLEWVGRIRSKSNNYATAYAASVKGRFTISRDDSKSTAYLQMDSLKTEDTAVYYCVGGGDFWGQGTLVTVSS

SEQ ID NO: 117 - protein, 115 aa, synthetic construct
  REGION 1..115: note = heavy chain
LVQLVESGGGLVQPGGSLKLSCAASGFTFNTYAMHWVRQASGKGLEWVGRIRSKSNNYATAYAASVKGRFTISRDDSKNMAYLQMDSLKTEDTAVYYCVGGGDFWGQGTLVTVSS

SEQ ID NO: 118 - protein, 115 aa, synthetic construct
  REGION 1..115: note = heavy chain
LVQLVESGGGLVQPGGSLKLSCAASGFTFNTYAMHWVRQASGKGLEWVGRIRSKSNNYATAYAASVKGRFTISRDDSKNTVYLQMDSLKTEDTAVYYCVGGGDFWGQGTLVTVSS

SEQ ID NO: 119 - protein, 115 aa, synthetic construct; identical to SEQ ID NO: 13
  REGION 1..115: note = heavy chain

SEQ ID NO: 120 - protein, 115 aa, synthetic construct
  REGION 1..115: note = heavy chain
LVQVVESGGGLVQPGGSLKLSCAASGFTFNTYAMNWVRQASGKGLEWVGRIRSKSNNYATYYAASVKGRFTISRDDSKSMAYLQMDSLKTEDTAVYYCVGGGDFWGQGTLVTVSS

SEQ ID NO: 121 - protein, 115 aa, synthetic construct
  REGION 1..115: note = heavy chain
LVQVVESGGGLVQPGGSLKLSCAASGFTFNTYAMNWVRQASGKGLEWVARIRSKSNNYATYYAASVKGRFTISRDDSKSMAYLQMDSLKTEDTAVYYCVGGGDFWGQGTLVTVSS

SEQ ID NO: 122 - protein, 107 aa, synthetic construct
  REGION 1..107: note = light chain
DIQMTQSPSSLSASLGERVSLTCRASQEISVYLSWFQQKPDGTIKRLIYGAFTLDSGVPKRFSGSRSGSDYSLTISSLESEDFADYYCLQYVRYPWTFGGGTKLEIK

SEQ ID NO: 123 - protein, 107 aa, synthetic construct
  REGION 1..107: note = light chain
DIQMTQSPSSLSASVGDRVTITCRASQEISVYLGWYQQKPGKAPKRLIYGAFKLQSGVPSRFSGSRSGTEFTLTISSLQPEDFATYYCLQYVRYPWTFGGGTKVEIK

SEQ ID NO: 124 - protein, 107 aa, synthetic construct; identical to SEQ ID NO: 123
  REGION 1..107: note = light chain

SEQ ID NO: 125 - protein, 107 aa, synthetic construct
  REGION 1..107: note = light chain
DIQMTQSPSSLSASVGDRVTITCRASQEISVYLSWFQQKPGKAIKRLIYGAFTLQSGVPSRFSGSRSGSEYTLTISSLQPEDFATYYCLQYVRYPWTFGGGTKVEIK

SEQ ID NO: 126 - protein, 107 aa, synthetic construct
  REGION 1..107: note = ligh chain
DIQMTQSPSSLSASVGDRVTITCRASQEISVYLSWFQQKPGKAPKRLIYGAFKLQSGVPSRFSGSRSGTEYTLTISSLQPEDFATYYCLQYVRYPWTFGGGTKVEIK

SEQ ID NO: 127 - protein, 107 aa, synthetic construct
  REGION 1..107: note = light chain
DIQMTQSPSSLSASVGDRVTITCRASQEISVYLSWYQQKPGKAPKRLIYGAFKLQSGVPSRFSGSRSGTEFTLTISSLQPEDFATYYCLQYVRYPWTFGGGTKVEIK

SEQ ID NO: 128 - protein, 107 aa, synthetic construct; identical to SEQ ID NO: 14
  REGION 1..107: note = light chain

SEQ ID NO: 129 - protein, 107 aa, synthetic construct
  REGION 1..107: note = light chain
DIQMTQSPSSLSASVGDRVTITCRASQEISVYLGWYQQKPGKAIKRLIYGAFKLQSGVPSRFSGSRSGTEFTLTISSLQPEDFATYYCLQYVRYPWTFGGGTKVEIK

SEQ ID NO: 130 - protein, 107 aa, synthetic construct
  REGION 1..107: note = light chain
DIQMTQSPSSLSASVGDRVTITCRASQEISVYLGWYQQKPGKAPKRLIYGAFTLQSGVPSRFSGSRSGTEFTLTISSLQPEDFATYYCLQYVRYPWTFGGGTKVEIK

SEQ ID NO: 131 - protein, 107 aa, synthetic construct
  REGION 1..107: note = light chain
DIQMTQSPSSLSASVGDRVTITCRASQEISVYLGWYQQKPGKAPKRLIYGAFKLQSGVPSRFSGSRSGSEFTLTISSLQPEDFATYYCLQYVRYPWTFGGGTKVEIK

SEQ ID NO: 132 - protein, 107 aa, synthetic construct
  REGION 1..107: note = light chain
DIQMTQSPSSLSASVGDRVTITCRASQEISVYLGWYQQKPGKAPKRLIYGAFKLQSGVPSRFSGSRSGTEYTLTISSLQPEDFATYYCLQYVRYPWTFGGGTKVEIK

SEQ ID NO: 133 - protein, 107 aa, synthetic construct
  REGION 1..107: note = light chain
DIQMTQSPSSLSASVGDRVTITCRASQEISVYLGWYQQKPGKAPKRLIYGAFSLQSGVPSRFSGSRSGTEFTLTISSLQPEDFATYYCLQYVRYPWTFGGGTKVEIK

SEQ ID NO: 134 - protein, 107 aa, synthetic construct
  REGION 1..107: note = light chain
DIQMTQSPSSLSASVGDRVTITCRASQEISVYLSWFQQKPGKAIKRLIYGAFKLQSGVPSRFSGSRSGTEYTLTISSLQPEDFATYYCLQYVRYPWTFGGGTKVEIK

SEQ ID NO: 135 - protein, 107 aa, synthetic construct; identical to SEQ ID NO: 18
  REGION 1..107: note = light chain

SEQ ID NO: 136 - protein, 107 aa, synthetic construct; identical to SEQ ID NO: 15
  REGION 1..107: note = light chain

SEQ ID NO: 137 - protein, 107 aa, synthetic construct; identical to SEQ ID NO: 16
  REGION 1..107: note = light chain

SEQ ID NO: 138 - protein, 107 aa, synthetic construct; identical to SEQ ID NO: 17
  REGION 1..107: note = light chain

SEQ ID NO: 139 - DNA, 41 bp, synthetic construct; identical to SEQ ID NO: 58
  misc_feature 1..41: note = primer
  source 1..41: mol_type = other DNA

SEQ ID NO: 140 - DNA, 43 bp, synthetic construct
  misc_feature 1..43: note = primer
  source 1..43: mol_type = other DNA
tgtaaaacgacggccagtatgacattgaacatgctgttggggc

SEQ ID NO: 141 - DNA, 33 bp, synthetic construct
  misc_feature 1..33: note = primer
  source 1..33: mol_type = other DNA
gatctccgtgtacctgagctggtatcagcagaa

SEQ ID NO: 142 - DNA, 321 bp, synthetic construct
  misc_feature 1..321: note = MoTau01_VK
  source 1..321: mol_type = other DNA
gacatccagatgacccagtctccatcctccttatctgcctctctgggagaaagagtcagtctcacttgtcgggcaagtcaggaaattagtgtttacttaagctggtttcagcagaaaccagatggaactattaaacgcctgatctacggcgcattcactttagattctggtgtcccaaaaaggttcagtggcagtaggtctgggtcagattattctctcaccatcagcagccttgagtctgaagattttgcagactattactgtctacaatatgttaggtatccgtggacgttcggtggaggcaccaagttggaaatcaaa

SEQ ID NO: 143 - DNA, 345 bp, synthetic construct
  misc_feature 1..345: note = MoTau01_VH
  source 1..345: mol_type = other DNA
gaggtgcaggttgttgagtctggtggaggattggtgcagcctaaagggtcattgaaactctcatgtgcagcctctggattcaccttcaatacctacgccatgaactgggtccgccaggctccaggaaagggtttggaatgggttgctcgcataagaagtaaaagtaataattatgcgacatattatgccgattcagtgaaagacaggttcaccatctccagagatgattcacaaagcatggtatatctgcaaatgaacaacttgaaaactgaggacacagccatgtattattgtgtgggagggggtgacttctggggccaaggcaccgctctcacagtctcctca

SEQ ID NO: 144 - DNA, 440 bp, synthetic construct
  misc_feature 1..440: note = MoTau01 VK
  source 1..440: mol_type = other DNA
tccttgacacgcgtctcgggaagcttgccgccaccatggaagccccagcgcagcttctcttcctcctgctactctggctccctgataccaccggagatatccagatgacacagtctccttctagcctgtccgcctctctgggagagagggtgtccctgacctgtagagcatcccaggagatcagcgtgtacctgagctggtttcagcagaagcccgacggcaccatcaagaggctgatctatggcgccttcacactggattctggcgtgcctaagcggtttagcggctccagatctggcagcgactactccctgacaatctctagcctggagtctgaggacttcgccgattactattgcctgcagtacgtgagatatccctggaccttcggcggcggcacaaagctggagatcaagagaactgtggcagcaccaagcgtc

SEQ ID NO: 145 - protein, 135 aa, synthetic construct
  REGION 1..135: note = MoTau01 VK
MEAPAQLLFLLLLWLPDTTGDIQMTQSPSSLSASLGERVSLTCRASQEISVYLSWFQQKPDGTIKRLIYGAFTLDSGVPKRFSGSRSGSDYSLTISSLESEDFADYYCLQYVRYPWTFGGGTKLEIKRTVAAPSV

SEQ ID NO: 146 - DNA, 551 bp, synthetic construct
  misc_feature 1..551: note = MoTau01VH codon optimised
  source 1..551: mol_type = other DNA
tccttgacacgcgtctcgggaagcttgccgccaccatggaagccccagcgcagcttctcttcctcctgctactctggctccctgataccaccggagaggtgcaggtggtggagagcggcggcggcctggtgcagcccaagggcagcctgaagctgtcctgtgccgcctctggcttcacctttaacacatatgccatgaattgggtgcggcaggcacctggcaagggactggagtgggtggcaagaatcagatctaagagcaacaattacgccacctactatgccgacagcgtgaaggataggttcacaatctcccgcgacgattcccagtctatggtgtatctgcagatgaacaattctgaagaccgaggacacagccatgtactattgcgtgggcggcggcgacttttggggacagggcaccgccctgacagtgagctccgcctccaccaagggcccatccgtcttccccctggcgccctgctccaggagcacctccgagagcacagccgccctgggctgcctggtcaaggactacttccccgaaccggt

SEQ ID NO: 147 - protein, 172 aa, synthetic construct
  REGION 1..172: note = MoTau01 VH codon opimised
MEAPAQLLFLLLLWLPDTTGEVQVVESGGGLVQPKGSLKLSCAASGFTFNTYAMNWVRQAPGKGLEWVARIRSKSNNYATYYADSVKDRFTISRDDSQSMVYLQMNNLKTEDTAMYYCVGGGDFWGQGTALTVSSASTKGPSVFPLAPCSRSTSESTAALGCLVKDYFPEPV

SEQ ID NO: 148 - DNA, 345 bp, synthetic construct
  misc_feature 1..345: note = Tau01 HA
  source 1..345: mol_type = other DNA
ctggtgcagctggtggaaagcggcggcggcctggtgcagcctggcggctccctgaagctgtcttgtgcagcaagcggcttcacctttaacacatatgcaatgcactgggtgcggcaggcatctggcaagggactggagtgggtgggccggatcagatctaagagcaacaattatgcaacagcatatgcagcatctgtgaagggcaggttcaccatctcccgcgacgattctaagaatacagcctatctgcagatggactccctgaagaccgaggatacagccgtgtactattgcgtgggcggcggcgacttttggggacagggcaccctggtgacagtgagctcc

SEQ ID NO: 149 - DNA, 345 bp, synthetic construct
  misc_feature 1..345: note = Tau01 HB
  source 1..345: mol_type = other DNA
gaggtgcaggtggtggaaagcggcggcggcctggtgcagcctggcggcagcctgaagctgtcctgtgcagcatctggcttcacctttaacacatacgccatgaattgggtgcggcaggcatctggcaagggactggagtgggtggcccggatcagatctaagagcaacaattacgccacctactatgccgacagcgtgaagggcaggttcacaatctcccgcgacgattccaagtctatggtgtatctgcagatggactccctgaagaccgaggatacagccgtgtactattgcgtgggcggcggcgacttttggggacagggcaccctggtgacagtgagctcc

SEQ ID NO: 150 - DNA, 345 bp, synthetic construct
  misc_feature 1..345: note = Tau01 HC
  source 1..345: mol_type = other DNA
ctggtgcagctggtggaaagcggcggcggcctggtgcagcctggcggcagcctgaagctgtcctgtgcagcatctggcttcacctttaacacatacgccatgaattgggtgcggcaggcatctggcaagggactggagtgggtgggccggatcagatctaagagcaacaattacgccacctactatgccgccagcgtgaagggcaggttcacaatctcccgcgacgattccaagtctatggcctatctgcagatggactccctgaagaccgaggatacagccgtgtactattgcgtgggcggcggcgacttttggggacagggcaccctggtgacagtgagctcc

SEQ ID NO: 151 - DNA, 321 bp, synthetic construct
  misc_feature 1..321: note = Tau01 KA
  source 1..321: mol_type = other DNA
gacatccagatgacccagtctccaagctccctgtccgcctctgtgggcgatagggtgaccatcacatgccgcgccagccaggagatctccgtgtacctgggctggtatcagcagaagcccggcaaggcccctaagcggctgatctacggcgccttcaagctgcagtccggagtgccaagccggttcagcggatccagatctggcaccgagtttaccctgacaatctctagcctgcagccagaggacttcgccacatactattgtctgcagtacgtgaggtatccctggaccttcggcggcggcacaaaggtggagatcaag

SEQ ID NO: 152 - DNA, 321 bp, synthetic construct
  misc_feature 1..321: note = Tau01 KB
  source 1..321: mol_type = other DNA
gacatccagatgacacagtctcctagctccctgtccgcctctgtgggcgatagggtgaccatcacatgccgcgcctcccaggagatcagcgtgtacctgagctggttccagcagaagccaggcaaggccatcaagcggctgatctatggagccttcaccctgcagtctggagtgccatctcggtttagcggctccagatctggcagcgagtacaccctgacaatctctagcctgcagcccgaggacttcgccacatactattgtctgcagtacgtgaggtatccttggaccttcggcggcggcacaaaggtggagatcaag

SEQ ID NO: 153 - DNA, 321 bp, synthetic construct
  misc_feature 1..321: note = Tau01 KC
  source 1..321: mol_type = other DNA
gacatccagatgacccagtctcctagctccctgtccgcctctgtgggcgatagggtgaccatcacatgccgcgccagccaggagatctccgtgtacctgtcttggttccagcagaagcccggcaaggcccctaagcggctgatctatggagccttcaagctgcagtctggagtgccatcccggtttagcggatccagatctggcaccgagtacaccctgacaatctctagcctgcagccagaggacttcgccacatactattgtctgcagtacgtgaggtatccctggaccttcggcggcggcacaaaggtggagatcaag